(19) United States
(12) Patent Application Publication
(10) Pub. No.: US 2011/0256134 A1
(43) Pub. Date: Oct. 20, 2011

(54) METHODS OF TREATMENT USING ANTI-OXIDIZED LDL ANTIBODIES

(76) Inventors: Stuart Bunting, Half Moon Bay, CA (US); Sherron Bullens, El Granada, CA (US); Roland Carlsson, Lund (SE); Anne-Christine Carlsson, Lund (SE); Björn Frendéus, Landskrona (SE); Steven Glazer, Frederiksberg (DK); Kevin Grove, Beaverton, OR (US); Nick Van Bruggen, San Carlos, CA (US)

(21) Appl. No.: 12/870,678

(22) Filed: Aug. 27, 2010

Related U.S. Application Data
(60) Provisional application No. 61/238,114, filed on Aug. 28, 2009.

(51) Int. Cl. A61K 39/395; A61P 9/00; A61P 3/00; A61P 3/10; A61P 5/50; A61P 29/00

(52) U.S. Cl. 424/135.1; 424/158.1; 424/139.1; 424/133.1

(57) ABSTRACT

The present invention relates to methods and compositions for increasing insulin sensitivity comprising the administration of anti-oxidized LDL antibodies.

### CROSS-REFERENCE TO RELATED APPLICATIONS

This application claims priority benefit to U.S. Provisional Application Ser. No. 61/238,114, filed Aug. 28, 2009, the entirety of which is hereby incorporated by reference.

### FIELD

The present invention concerns methods for increasing insulin sensitivity in a subject using anti-oxidized low-density lipoprotein (LDL) antibodies.

### BACKGROUND

Metabolic syndrome is a complex disease, characterized by the American Heart Association by the following abnormalities: abdominal obesity, atherogenic dyslipidemia, hypertension, insulin resistance with or without glucose intolerance, proinflammatory state and prothrombotic state (Grundy et al., “DEFINITION OF METABOLIC SYNDROME” Circulation, 2004, V109, pages 433-438, Document Number DOI: 10.1161/01.CIR.0000111245.75752.C6 available at www.circulationaha.org, herein fully incorporated by reference). It is generally recognized in the art that people with three or more of the above symptoms can be considered to have metabolic syndrome. The American Heart Association estimates that about 20 to 25 percent of U.S. adults have metabolic syndrome.

A significant population of people with metabolic syndrome are pre-diabetic and have blood glucose levels that are higher than normal, but not high enough for a diagnosis of diabetes and are at risk of developing type 2 diabetes, heart disease, and stroke. Pre-diabetes is becoming more common in the United States. The U.S. Department of Health and Human Services estimates that at least 57 million U.S. adults ages 20 or older had pre-diabetes in 2007. Those with pre-diabetes are likely to develop type 2 diabetes within 10 years, unless they take steps to prevent or delay diabetes.

Diabetes itself affects an estimated 23.6 million people in the United States—7.8 percent of the population. Of those, 17.9 million have been diagnosed, and 5.7 million have not yet been diagnosed. In 2007, about 1.6 million people ages 20 or older were diagnosed with diabetes. Diabetes is caused by occurrence of abnormal metabolisms of glucose, protein and lipid due to a deficiency or insufficiency of the actions of insulin.

Typical signs of diabetes include an abnormal increase in the serum glucose level over the normal range of the glucose level and an excretion of glucose in the urine. Diabetes mellitus is a disease which affects millions people in the United States and, although a heterogeneous disorder, it generally is classified within three major categories, i.e., Type I diabetes, Type II diabetes, and gestational diabetes. About 80% of all diabetics in the United States are in the Type II category. This type of diabetes is characterized by both impaired insulin secretion and insulin resistance. The majority of patients are obese adults and loss of weight can restore normoglycemia in some cases. However, this type of diabetes can also occur in the non-obese adults and in children.

There is an urgent need to find a method of increasing insulin sensitivity in patients to improve methods of preventing or treating metabolic syndrome or a symptom or condition associated with metabolic syndrome such as pre-diabetes and diabetes.

### BRIEF SUMMARY

Provided herein are methods of increasing insulin sensitivity in a subject comprising administering to the subject an effective amount of a composition comprising an antibody that selectively binds to an epitope of oxidized low density lipoprotein (LDL).

Further provided herein are uses of an antibody that selectively binds to an epitope of oxidized low density lipoprotein (LDL) in the manufacture of a medicament for increasing insulin sensitivity in a subject.

Provided also herein are medicaments comprising an antibody that selectively binds to an epitope of oxidized low density lipoprotein (LDL) for increasing insulin sensitivity in a subject.

Also provided herein are antibodies that selectively bind to an epitope of oxidized LDL for increasing insulin sensitivity.

In some embodiments of any of the methods, uses, medicaments, and antibodies provided herein, the epitope of oxidized LDL comprises an epitope of oxidized ApoB-100. In some embodiments, the epitope of oxidized ApoB-100 is selected from the group consisting of the peptide sequences in Table 1. In some embodiments, the epitope of oxidized ApoB-100 is selected from the group consisting of SEQ ID NO:1-SEQ ID NO:38. In some embodiments, the epitope of oxidized ApoB-100 is P45 (amino acid residues 661-680-IEIGLEGKGFEPTLEALFGK; SEQ ID NO:32), P143 (amino acid residues 2131-2150-IALDDAKINFNEKLSQLQTY; SEQ ID NO:13), P210 (amino acid sequence KTTKQSFDLSVKAQYKKNKH; SEQ ID NO:14), and/or P129 (amino acid sequence GSTSHHLVSRKSISAALEHK; SEQ ID NO:16).

In some embodiments of any of the methods, uses, medicaments, and antibodies provided herein, the antibody is a monoclonal antibody. In some embodiments, the monoclonal antibody is an IgG1 antibody. In some embodiments, the monoclonal antibody is a humanized antibody or a human antibody. In some embodiments, the monoclonal antibody comprises a light chain variable domain comprising (i) CDR-L1 comprising sequence CSGSNTNIGKNYVS (SEQ ID NO:39); (ii) CDR-L2 comprising sequence ANSNRPS (SEQ ID NO:40); and/or (iii) CDR-L3 comprising sequence CASWDASLNGWV (SEQ ID NO:41). In some embodiments, the monoclonal antibody comprises a heavy chain variable domain comprising (i) CDR-H1 comprising sequence FSNAWMSWVRQAPG (SEQ ID NO:42); (ii) CDR-H2 comprising sequence SSISVGGHRTYYADSVKGR (SEQ ID NO:43); and/or (iii) CDR-H3 comprising sequence ARIRVGPSGGAFDY (SEQ ID NO:44). In some embodiments, the monoclonal antibody comprises a light chain variable domain comprising (i) CDR-L1 comprising sequence CSGSSSNIGNNAVN (SEQ ID NO:45); (ii) CDR-L2 comprising sequence GNDRRPS (SEQ ID NO:46); and/or (iii) CDR-L3 comprising sequence CQTWGTGRGV (SEQ ID NO:47). In some embodiments, the monoclonal antibody comprises a heavy chain variable domain comprising (i) CDR-H1 comprising sequence FSDYYMSWVRQAPG (SEQ ID NO:48); (ii) CDR-H2 comprising sequence SGVSWNGSRTHYADSVKGR (SEQ ID NO:49); and/or (iii) CDR-H3 comprising sequence ARAARYSYYYYGMDV (SEQ ID NO:50). In some embodiments, the monoclonal antibody comprises a light chain variable domain comprising (i) CDR-L1 comprising sequence CSGSSSNIGNNYVS (SEQ ID NO:127); (ii) CDR-L2 comprising sequence SNNQRPS (SEQ ID NO:128); and/or (iii) CDR-L3 comprising sequence CAAWDDSLSHWL (SEQ ID NO:129). In some embodiments, the monoclonal antibody comprises a heavy chain variable domain comprising (i) CDR-H1 comprising sequence FSNAWMSWVRQVPG (SEQ ID NO:130); (ii) CDR-H2 comprising sequence STLGGSGGGSTYYADSVKGR (SEQ ID NO:131); and/or (iii) CDR-H3 comprising sequence AKLGGRSRYGRWPRQFDY (SEQ ID NO:132). In some embodiments, the monoclonal antibody comprises a light chain variable domain comprising (i) CDR-L1 comprising sequence CSGSSSNIGSNYVS (SEQ ID NO:133); (ii) CDR-L2 comprising sequence GNYNRPS (SEQ ID NO:134); and/or (iii) CDR-L3 comprising sequence CAAWDDSLSGWV (SEQ ID NO:135). In some embodiments, the monoclonal antibody comprises a heavy chain variable domain comprising (i) CDR-H1 comprising sequence FSSYWMSWVRQAPG (SEQ ID NO:136); (ii) CDR-H2 comprising sequence SSISGSGRRTYYADSVQGR (SEQ ID NO:137); and/or (iii) CDR-H3 comprising sequence ARLVSYGSGSFGFDY (SEQ ID NO:138). In some embodiments, the monoclonal antibody a light chain variable domain comprising (i) CDR-L1 comprising sequence CSGRSSNIGNSYVS (SEQ ID NO:139); (ii) CDR-L2 comprising sequence RNNQRPS (SEQ ID NO:140); and/or (iii) CDR-L3 comprising sequence CAGWDDTLRAWV (SEQ ID NO:141). In some embodiments, the monoclonal antibody comprises a heavy chain variable domain comprising (i) CDR-H1 comprising sequence FRDYYVSWIRQAPG (SEQ ID NO:142); (ii) CDR-H2 comprising sequence SSISGSGGRTYYADSVEGR (SEQ ID NO:143); and/or (iii) CDR-H3 comprising sequence ARVSALRRPMTTVTTYWFDP (SEQ ID NO:144).

In some embodiments of any of the methods, uses, medicaments, and antibodies provided herein, the monoclonal antibody is a human antibody. In some embodiments, the human antibody comprises a heavy chain variable domain comprising a sequence selected from the group consisting of SEQ ID NO:64, SEQ ID NO:68, SEQ ID NO:72, SEQ ID NO:76, SEQ ID NO:80, SEQ ID NO:84, SEQ ID NO:88, SEQ ID NO:92, SEQ ID NO:96, SEQ ID NO:100, SEQ ID NO:104, SEQ ID NO:108, SEQ ID NO:112, SEQ ID NO:116, SEQ ID NO:120, and SEQ ID NO:124. In some embodiments, the human antibody comprises a light chain variable domain comprising a sequence selected from the group consisting of SEQ ID NO:66, SEQ ID NO:70, SEQ ID NO:74, SEQ ID NO:78, SEQ ID NO:82, SEQ ID NO:86, SEQ ID NO:90, SEQ ID NO:94, SEQ ID NO:98, SEQ ID NO:102, SEQ ID NO:106, SEQ ID NO:110, SEQ ID NO:114, SEQ ID NO:118, SEQ ID NO:122, and SEQ ID NO:126. In some embodiments, the human antibody comprises a heavy chain variable domain comprising SEQ ID NO:104 and a light chain variable domain comprising SEQ ID NO:106. In some embodiments, the human antibody comprises a heavy chain variable domain comprising SEQ ID NO:68 and a light chain variable domain comprising SEQ ID NO:70. In some embodiments, the human antibody comprises a heavy chain variable domain comprising SEQ ID NO:96 and a light chain variable domain comprising SEQ ID NO:98. In some embodiments, the human antibody comprises a heavy chain variable domain comprising SEQ ID NO:72 and a light chain variable domain comprising SEQ ID NO:74. In some embodiments, the human antibody comprises a heavy chain variable domain comprising SEQ ID NO:76 and a light chain variable domain comprising SEQ ID NO:78.

In some embodiments of any of the methods, uses, medicaments, and antibodies provided herein, the humanized antibody or the human antibody is an antigen binding fragment. In some embodiments, the antigen binding fragment is selected from the group consisting of a Fab fragment, a Fab′ fragment, a F(ab′)₂ fragment, a scFv, a Fv, and a diabody. In some embodiments, the antibody further reduces inflammation. In some embodiments, the inflammation is associated with diabetes. In some embodiments, the antibody further reduces levels of an inflammatory marker, wherein the inflammatory marker is selected from the group consisting of IL-1β, IL-15, EN-RAGE, MCP-1, IL-6, and TNF-α. In some embodiments, the inflammatory marker is IL-1β, IL-15, EN-RAGE, and TNF-α.

In some embodiments of any of the methods, uses, medicaments, and antibodies provided herein, the subject has metabolic syndrome. In some embodiments, the subject is at risk for developing metabolic syndrome. In some embodiments, the subject has one or more characteristics selected from the group consisting of (a) waist circumference of about 102 cm or more in men and about 88 cm or more in women, (b) fasting triglycerides of about 150 mg/dL or more, (c) a fasting glucose of about 95 mg/dL or higher, and (d) high levels of oxidized LDL. In some embodiments, the subject further has inflammation associated with diabetes. In some embodiments, the subject has a blood glucose level of about 95 mg/dL or higher after an overnight fast. In some embodiments, the subject has a blood glucose level of about 126 mg/dL or higher after an overnight fast. In some embodiments, the subject has a blood glucose level of about 140 mg/dL or higher after a two-hour oral glucose tolerance test. In some embodiments, the subject has a blood glucose level of about 200 mg/dL or higher after a two-hour oral glucose tolerance test. In some embodiments, the subject has pre-diabetes. In some embodiments, the subject has diabetes. In some embodiments, the diabetes is selected from the group consisting of type-I diabetes, type-II diabetes, and gestational diabetes. In some embodiments, the diabetes is type-II diabetes. In some embodiments, the subject has a cardiovascular disease or coronary heart disease. In some embodiments, the cardiovascular disease or coronary heart disease is associated with diabetes. In some embodiments, the subject has atherosclerosis. In some embodiments, the atherosclerosis is associated with diabetes.

In some embodiments of any of the methods, uses, medicaments, and antibodies described herein, the increase in insulin sensitivity is for treating pre-diabetes in a subject. In some embodiments, the increase in insulin sensitivity is for treating diabetes in a subject. In some embodiments, the diabetes is selected from the group consisting of type-I diabetes, type-II diabetes, and gestational diabetes. In some embodiments, the diabetes is type-II diabetes. In some embodiments, the increase in insulin sensitivity is for treating a cardiovascular disease or coronary heart disease in a subject. In some embodiments, the cardiovascular disease or coronary heart disease is associated with diabetes. In some embodiments, the increase in insulin sensitivity is for treating atherosclerosis in a subject. In some embodiments, the atherosclerosis is associated with diabetes.

In some embodiments of any of the methods, uses, medicaments, and antibodies provided herein, the method, use, or medicament further comprises a second therapeutic agent. In some embodiments, the second therapeutic agent is administered. In some embodiments, the second therapeutic agent is insulin. In some embodiments, the second therapeutic agent is a statin.

### BRIEF DESCRIPTION OF THE DRAWINGS

FIG. 1 shows a schematic of the experimental schedule and list of parameters evaluated.

FIG. 2A shows the Area Under the Curve (AUC; area under the serum concentration-time curve) results for IVGTT (intravenous glucose tolerance test) for insulin based on animal identification number.

FIG. 2B shows the plasma insulin levels (uIU/mL) over time as determined by IVGTT based on animal identification number.

FIG. 3A shows mean plasma insulin (uIU/mL) for age matched, HFD-elevated insulin, HFD-normal insulin at baseline, after fructose, and after 2D03 treatment for 12 weeks using IVGTT.

FIG. 3B shows glucose tolerance test of animals fed a high fat or normal diet before and after treatment with 2D03 as measured by insulin AUC using IVGTT.

FIG. 4 shows the level of EN-RAGE in ng/mL over time as measured by Rules-Based Medicine based on animal identification number.

FIG. 5 shows the level of FGF-basic in pg/mL over time as measured by Rules-Based Medicine based on animal identification number.

FIG. 6 shows the level of IL-13 in pg/mL over time as measured by Rules-Based Medicine based on animal identification number.

FIG. 7A shows the level of IL-15 in μg/mL over time as measured by Rules-Based Medicine based on animal identification number.

FIG. 7B shows the level of interferon-γ in pg/mL over time as measured by Rules-Based Medicine based on animal identification number.

FIG. 8A shows the level of interleukin-1β in pg/mL over time as measured by ELISA based on animal identification number.

FIG. 8B shows the level of TNF-α in pg/mL over time as measured by ELISA based on animal identification number.

FIG. 9A shows the level of IL-6 in pg/mL over time as measured by ELISA based on animal identification number.

FIG. 9B shows the level of IL-6 in pg/mL over time as measured by Rules-Based Medicine based on animal identification number.

FIG. 10 shows T-cell subpopulations during the pre-treatment period (baseline period), the treatment period, and wash period (post-treatment period). White bars represent control animals (CTR) while black bars represent animals on a high-fat and fructose-supplement diet (HFD).

### DETAILED DESCRIPTION

### I. Methods of Treatment and Uses

Provided herein are methods of increasing insulin sensitivity in a subject comprising administering to the subject an effective amount of a composition comprising an anti-oxidized LDL antibody. Further provided herein are uses of an anti-oxidized LDL antibody in the manufacture of a medicament for increasing insulin sensitivity in a subject and medicaments comprising an antibody that selectively binds to an epitope of oxidized low density lipoprotein (LDL) for use in increasing insulin sensitivity.

In some embodiments of any of the methods and uses, insulin sensitivity is increased at least about any of 5%, 10%, 25%, 50%, 75%, 100%, 150%, or 200%. In some embodiments, insulin sensitivity is increased about any of 5%, 10%, 25%, 50%, 75%, 100%, 150%, or 200%. In some embodiments, insulin resistance is decreased. In some embodiments, insulin resistance is decrease at least about any of 5%, 10%, 25%, 50%, 75%, or 90%. In some embodiments, insulin resistance is decreased about any of 5%, 10%, 25%, 50%, 75%, or 90%. Insulin sensitivity may be increased compared to at the time of starting treatment. In some embodiments, insulin resistance may be decreased compared to at the time of starting treatment. As used herein, “at the time of starting treatment” refers to the time period at or prior to the first exposure to an anti-oxidized LDL antibody. In some embodiments, “at the time of starting treatment” is about any of one year, nine months, six months, three months, second months, or one month prior to an anti-oxidized LDL antibody. In some embodiments, “at the time of starting treatment” is immediately prior to coincidental with the first exposure to an anti-oxidized LDL antibody.

In some embodiments of any of the methods and uses, insulin sensitivity and/or insulin resistance is tested using one or more of the following tests: hyperinsulinemic euglycemic clamp, insulin suppression test (IST), insulin tolerance test (ITT), continuous infusion of glucose with model assessment (CIGMA), intravenous glucose tolerance test (IVGTT), oral glucose tolerance test (OGTT), HOMA model, fasting insulin (I0), glucose/insulin ratio (G/I ratio), and/or insulin sensitivity index (ISI).

In some embodiments of any of the methods and uses, insulin sensitivity is measured by hyperinsulinemic euglycemic clamp, which measures the amount of glucose necessary to compensate for an increased insulin level without causing hypoglycemia. The rate of glucose infusion during the last 30 minutes of the test determines insulin sensitivity. If high levels (7.5 mg/min or higher) are required, the patient is insulin-sensitive. Very low levels (4.0 mg/min or lower) indicate that the body is resistant to insulin action. Levels between 4.0 and 7.5 mg/min may suggest “impaired glucose tolerance”, an early sign of insulin resistance. A subject may have a rate of glucose infusion greater than about any of 9 mg/min, 8 mg/min, 7.5 mg/min, 7.0 mg/min, 6 mg/min, 5 mg/min, or 4 mg/min at the time of starting treatment. In some embodiments, insulin sensitivity may be increased in the subject as indicated by a rate of glucose infusion being less than about any of 7.5 mg/min, 7.0 mg/min, 6 mg/min, 5 mg/min, or 4 mg/min after exposure to an anti-oxidized LDL antibody.

In some embodiments of any of the methods and uses, insulin sensitivity is tested using the insulin suppression test (IST). Subjects with a steady-state plasma glucose (SSPG) level greater than 150 mg/dl are considered to be insulin-resistant. In some embodiments, the subject has an SSPG level of greater than about any of 150 mg/dL, 175 mg/dL, or 200 mg/dL at the time of starting treatment. In some embodiments, insulin sensitivity is increased in the subject as indicated by an SSPG level of less than 175 mg/dL, 150 mg/dL, or 125 mg/dL after exposure to an anti-oxidized LDL antibody.

In some embodiments of any of the methods and uses, insulin sensitivity is tested using a glucose tolerance test, such as an OGTT or IVGTT. In some embodiments, the subject has a blood glucose level of about 140 mg/dL or higher after a two-hour oral glucose tolerance test. In some embodiments, the subject has a blood glucose level of about 200 mg/dL or higher after a two-hour oral glucose tolerance test. In some embodiments, the subject has impaired glucose tolerance (IGT). In some embodiments, the subject has a blood glucose level of about any of 140 mg/dL or higher, 150 mg/dL or higher, 160 mg/dL or higher, 170 mg/dL or higher, 180 mg/dL or higher, 190 mg/dL or higher, or 200 mg/dL or higher 2 hours after administration of 75 g of glucose. In some embodiments, the subject has a blood glucose level of about any of 140 mg/dL, 150 mg/dL, 160 mg/dL, 170 mg/dL, 180 mg/dL, 190 mg/dL, or 200 mg/dL 2 hours after administration of 75 g of glucose.

In some embodiments of any of the methods and uses, glucose tracers are used. Glucose can be labeled with either stable or radioactive atoms. Commonly-used tracers are 3-3H glucose (radioactive), 6,6 2H-glucose (stable) and 1-13C Glucose (stable). Prior to beginning the hyperinsulinemic period, a 3 h tracer infusion enables one to determine the basal rate of glucose production. The plasma tracer concentrations enable the calculation of whole-body insulin-stimulated glucose metabolism, as well as the production of glucose by the body (i.e., endogenous glucose production).

In some embodiments of any of the methods and uses, the subject has metabolic syndrome. In some embodiments, the subject is at risk for developing metabolic syndrome. The subject may have one or more characteristics selected from the group consisting of (a) waist circumference of about 102 cm or more in men and about 88 cm or more in women, (b) fasting triglycerides of about 150 mg/dL or more, (c) a fasting glucose of about 95 mg/dL or higher, and (d) high levels of oxidized LDL. In some embodiments, the subject has a cholesterol level below about 40 mg/dL for men and below about 50 mg/dL for women. In some embodiments, the subject has a blood pressure level of about 130/85 or above or about 140/90 or above. In some embodiments, high levels of oxidized LDL are about any of oxidized-LDL greater than or equal to 0.5 nmol/mg, 0.6 nmol/mg, 0.7 nmol/mg, 0.8 nmol/mg, 0.9 nmol/mg, 1.0 nmol/mg apoprotein.

In some embodiments of any of the methods and uses, the fasting glucose level is about any of 95 mg/dL, 100 mg/dL, 105 mg/dL, 110 mg/dL, 115 mg/dL, 120 mg/dL, 125 mg/dL, or 130 mg/dL. In some embodiments, the fasting glucose level is about any of 95 mg/dL or higher, 100 mg/dL or higher, 105 mg/dL or higher, 110 mg/dL or higher, 115 mg/dL or higher, 120 mg/dL or higher, 125 mg/dL or higher, or 130 mg/dL or higher. The subject may have a blood glucose level of about 95 mg/dL or higher after an overnight fast. In some embodiments, the subject may have a blood glucose level of about 126 mg/dL or higher after an overnight fast.

In some embodiments of any of the methods and uses, administration to the subject of an effective amount of a composition comprising an antibody that selectively binds to an epitope of oxidized low density lipoprotein (LDL) increases glycemic control (improves glycemic control). In some embodiments of any of the methods and uses, administration to the subject of an effective amount of a composition comprising an antibody that selectively binds to an epitope of oxidized low density lipoprotein (LDL) increases lipid catabolism and overall energy expenditure. In some embodiments, the increase is determined by comparison of level prior to treatment with the antibody and after treatment with the antibody.

In some embodiments of any of the methods and uses, the subject has pre-diabetes. In some embodiments, the subject has diabetes. In some embodiments, the diabetes is selected from the group consisting of type-I diabetes, type-II diabetes, and gestational diabetes. The diabetes may type-II diabetes. In some embodiments, the methods or uses provided herein are useful in treating, preventing or delaying the progression of diabetes. The diabetes may be selected from the group consisting of type-I diabetes, type-II diabetes, and gestational diabetes. In some embodiments, the methods and uses provided herein are useful in preventing or delaying the progression of pre-diabetes to diabetes.

In some embodiments of any of the methods and uses, the subject has a cardiovascular disease or coronary heart disease. The cardiovascular disease or coronary heart disease may be associated with diabetes. The cardiovascular disease or coronary heart disease may also be associated with insulin resistance and metabolic syndrome. In some embodiments, the subject has atherosclerosis. The atherosclerosis may be associated with diabetes. The atherosclerosis may also be associated with insulin resistance and metabolic syndrome.

In some embodiments of any of the methods and uses, the subject further has inflammation. The inflammation may be associated with diabetes.

Provided herein also are methods of reducing inflammation in a subject comprising administering an effective amount of a composition comprising an anti-oxidized LDL antibody and/or uses of an anti-oxidized LDL antibody in the preparation of a medicament for use in reducing inflammation in a subject. In some embodiments, the methods and uses include reducing inflammation and increasing insulin sensitivity, as described above, in a subject comprising administering an effective amount of a composition comprising an anti-oxidized LDL antibody. In some embodiments, the inflammation is associated with diabetes. In some embodiments, the inflammation is reduced as evidenced by reduces levels of an inflammatory marker. The level of an inflammatory marker may be reduced about any of 5%, 10%, 25%, 50%, 75%, or 90%. In some embodiments, the level of an inflammatory marker is reduced greater than about any of 5%, 10%, 25%, 50%, 75%, or 90%. The inflammatory marker may be selected from the group consisting of FGF-basic, IL-1β, IL-15, IL-8, IL-13, IL-6, MCP-1, EN-RAGE, and TNF-α. In some embodiments, the inflammatory marker is selected from the group consisting of IL-1β, IL-15, EN-RAGE, and TNF-α. The inflammation or levels of an inflammatory marker may be reduced compared to inflammation or levels of an inflammatory marker at the time of starting treatment with the anti-oxidized LDL antibody.

In some embodiments of any of the methods and uses, the methods and uses may also comprise more than one active compound, preferably those with complementary activities that do not adversely affect each other. For example, it may be desirable to further administer an anti-inflammatory agent, an anti-diabetic agent, and/or cholesterol lowering drug of the “statin” class in the formulation. In some embodiments, the method and use comprises administering a second active agent. In some embodiments, the method and use comprises administering a second active agent, wherein the second active agent is insulin. The insulin is rapid acting, short acting, regular acting, intermediate acting, or long acting insulin. In some embodiments, the insulin may be and/or comprises Humalog, Lispro, Novolog, Apidra, Humulin, Aspart, regular insulin, NPH, Lente, Ultralente, Lantus, Glargine, Levemir, or Detemir. In some embodiments, the method and/or use comprises administering a second active agent, wherein the second active agent is a statin. The statin may be and/or comprises Atorvastatin (e.g., Lipitor or Torvast), Cerivastatin (e.g., Lipobay or Baycol), Fluvastatin (e.g., Lescol or Lescol), Lovastatin (e.g., Mevacor, Altocor, or Altoprev) Mevastatin, Pitavastatin (e.g., Livalo or Pitava), Pravastatin (e.g., Pravachol, Selektine, or Lipostat) Rosuvastatin (e.g., Crestor), Simvastatin (e.g., Zocor or Lipex), Vytorin, Advicor, Besylate Caduet or Simcor.

In some embodiments of any of the methods and uses, the anti-oxidized LDL antibody can be administered by methods and routes well-known in the art such as intravenous administration, e.g., as a bolus or by continuous infusion over a period of time, by intramuscular, intraperitoneal, intracerobrospinal, subcutaneous, intra-articular, intrasynovial, intrathecal, oral, topical, or inhalation routes. When the methods and uses described herein include more than one active agent(s), the methods and uses may include administration of the anti-oxidized LDL antibody in combination with one or more other (i.e., further) active agent(s). Administration “in combination with” one or more other active agents includes simultaneous (concurrent), consecutive administration in any order, and sequentially in any order. The combined administration includes co-administration, using separate formulations or a single pharmaceutical formulation, consecutive administration in either order, and sequential administration in any order, wherein preferably there is a time period while both (or all) active agents simultaneously exert their biological activities. Preferably such combined therapy results in a synergistic therapeutic effect.

As used herein, “treatment” or “treating” is an approach for obtaining beneficial or desired results including clinical results. For purposes of this invention, beneficial or desired clinical results include, but are not limited to, one or more of the following: decreasing one or more symptoms resulting from the disease, diminishing the extent of the disease, stabilizing the disease (e.g., preventing or delaying the worsening of the disease), delay or slowing the progression of the disease, ameliorating the disease state, decreasing the dose of one or more other medications required to treat the disease, and/or increasing the quality of life.

As used herein, “delaying” the progression means to defer, hinder, slow, retard, stabilize, and/or postpone development of the disease. This delay can be of varying lengths of time, depending on the history of the disease and/or individual being treated.

In some embodiments of any of the methods and uses, the methods of treatment described herein ameliorate (e.g., reduce incidence of, reduce duration of, reduce or lessen severity of) of one or more symptoms of the disease.

A “subject” herein is a mammal. Mammals include, but are not limited to, humans, farm animals, sport animals, rodents, primates and certain pets. In some embodiments, the mammal is a human.

A “symptom” is any morbid phenomenon or departure from the normal in structure, function, or sensation, experienced by the subject.

The expression “effective amount” refers to an amount of the antibody (or other drug) that is effective for increasing insulin sensitivity and/or reducing inflammation (such as inflammation associated with diabetes). Such an effective amount will generally result in an improvement in the signs, symptoms or other indicators of insulin resistance and/or inflammation.

Reference to “about” a value or parameter herein includes (and describes) variations that are directed to that value or parameter per se. For example, description referring to “about X” includes description of “X”.’

As used herein and in the appended claims, the singular forms “a,” “or,” and “the” include plural referents unless the context clearly dictates otherwise. It is understood that aspects and variations of the invention described herein include “consisting” and/or “consisting essentially of” aspects and variations.

### II. Antibodies

Provided herein are anti-oxidized LDL antibodies for use in any of the methods and/or uses of increasing insulin sensitivity. In some embodiments, the anti-oxidized LDL antibodies bind to oxidized LDL.

Oxidized LDL contains several different epitopes that can be recognized by antibodies. LDL may undergo oxidative and degrading changes through a wide variety of different chemical reactions. These include reactions caused by different types of modifications caused by the activity of oxygen, enzymes (e.g., myeloperoxidase), metal ions (e.g., Fe²⁺ and Cu²⁺), free radicals and other types of chemical stress.

Some of the oxidized epitopes are found on the protein part of LDL (Yang et al., J. Lipid Res. 42(11):1891-6 (2001)) but others are modifications of lipids present in the LDL particle. Many oxidatively modified and biologically active phospholipids can be formed (Heery et al., J. Clin. Invest. 96(5):2322-30 (1995); Friedman et al., J. Biol. Chem. 277(9):7010-20 (2002); Watson et al., J. Biol. Chem. 274(35):34787-98 (1999)). Polyunsatured fatty acids are converted to fatty acid hydroperoxides, which rapidly form highly reactive products such as malondialdehyde and 4-hydroxynonenal (Smiley et al., J. Biol. Chem. 266(17):11104-10 (1991)). These type of intermediate products can go on to form covalent Schiff base and Michael-type products with the lysine in ApoB-100 protein of LDL. Reactive aldehydes can also be found in fatty acids attached via the ester bonds in the phosphatidylcholine moiety (Witztum & Berliner, Curr. Opin. Lipidol. 9(5):441-8 (1998)). Frequently found is phospholipid 1-palmitoyl-2-arachidonoylsn-glycero-3-phosphorylcholine (PAPC), a near terminal oxidation product that yields an aldehyde at a carbon of the sn-2 oxidized arachidonic acid, resulting in POVPC (1-palmitoyl-2-(5-oxo)valeroyl-sn-glycero-3-phosphorylcholine). POVPC can react with lysine and also with amine-containing phospholipids such as phosphatidylethanolamine and phosphatidylserine. The end result is a variety of oxidized lipid-protein and oxidized lipid-lipid adducts. Some of these oxidations are driven by enzymes such as secretory phospholipase (Leitinger et al., Arterioscler Thromb Vasc. Biol. 19(5):1291-8 (1999)). Other enzyme-formed changes such as nitration and addition of HOCL are performed by myeloperoxidase (Carr et al., Arterioscler Thromb Vasc. Biol. 20(7):1716-23 (2000)). All neoepitopes are to be considered as immunogenic and biologically active (McIntyre et al., J. Biol. Chem. 274(36):25189-92 (1999)). Also cryptic epitopes that are a result of an oxidized modification of the LDL particle but are not oxidized themselves, like phosphorylcholine and fragments of proteins, are hallmarks of oxidized LDL particles and such epitopes are targetable by immunotherapy.

In some embodiments, the anti-oxidized LDL antibody binds an epitope of oxidized LDL comprising an epitope of oxidized ApoB-100 (for example, NP_{—}000375.2). In some embodiments, the anti-oxidized LDL antibody bind to an epitope of oxidized ApoB-100 comprising the amino acid sequence of Table 1. In some embodiments, the anti-oxidized LDL antibody is capable of binding to a peptide consisting of the sequence of Table 1. In some embodiments, the anti-oxidized LDL antibody is capable of binding to an epitope of oxidized ApoB-100 selected from the group consisting of SEQ ID NO:1-38.

Each of the antibodies described in WO 2002/080954, WO 2004/030607, Schiopu et al., Circulation 110(14):2047-52 (2004), and WO 2007/025781 are examples of antibodies that may be used in the context of the present invention and are hereby incorporated by reference.

TABLE 1

| | | |
|---|---|---|
| PEPTIDE | | SEQ ID |
| NAME | PEPTIDE SEQUENCE | NO: |

Category A. High IgG, MDA-difference

| | | |
|---|---|---|
| P11. | FLDTVYGNCSTHFTVKTRKG | 1 |
| P25. | PQCSTHILQWLKRVHANPLL | 2 |
| P74. | VISIPRLQAEARSEILAHWS | 3 |

Category B. High IgM, no MDA-difference

| | | |
|---|---|---|
| P40. | KLVKEALKESQLPTVMDFRK | 4 |
| P68. | LKFVTQAEGAKQTEATMTFK | 5 |
| P94. | DGSLRHKFLDSNIKFSHVEK | 6 |
| P99. | KGTYGLSCQRDPNTGRLNGE | 7 |
| P100. | RLNGESNLRFNSSYLQGTNQ | 8 |
| P102. | SLTSTSDLQSGIIKNTASLK | 9 |
| P103. | TASLKYENYELTLKSDTNGK | 10 |
| P105. | DMTFSKQNALLRSEYQADYE | 11 |
| P177. | MKVKIIRTIDQMQNSELQWP | 12 |

Category C. High IgG, no MDA difference

| | | |
|---|---|---|
| P143. | IALDDAKINFNEKLSQLQTY | 13 |
| P210. | KTTKQSFDLSVKAQYKKNKH | 14 |

Category D. NHS/AHP, IgG-ak>2, MDA-difference

| | | |
|---|---|---|
| P1. | EEEMLENVSLVCPKDATRFK | 15 |
| P129. | GSTSHHLVSRKSISAALEHK | 16 |
| P148. | IENIDFNKSGSSTASWIQNV | 17 |
| P162. | IREVTQRLNGEIQALELPQK | 18 |
| P252. | EVDVLTKYSQPEDSLIPFFE | 19 |

Category E. NHS/AHP, IgM-ak>2, MDA-difference

| | | |
|---|---|---|
| P301. | HTFLIYITELLKKLQSTTVM | 20 |
| P30. | LLDIANYLMEQIQDDCTGDE | 21 |
| P31. | CTGDEDYTYLILRVIGNMGQ | 22 |
| P32. | GNMGQTMEQLTPELKSSILK | 23 |
| P33. | SSILKCVQSTKPSLMIQKAA | 24 |
| P34. | IQKAAIQALRKMEPKDKDQE | 25 |
| P100. | RLNGESNLRFNSSYLQGTNQ | 26 |
| P107. | SLNSHGLELNADILGTDKIN | 27 |
| P149. | WIQNVDTKYQIRIQIQEKLQ | 28 |
| P169. | TYISDWWTLAAKNLTDFAEQ | 29 |
| P236. | EATLQRIYSLWEHSTKNHLQ | 30 |

Category F. NHS/AHP, IgG-ak>0.5, no
MDA-difference

| | | |
|---|---|---|
| P10. | ALLVPPETEEAKQVLFLDTV | 31 |
| P45. | IEIGLEGKGFEPTLEALFGK | 32 |
| P111. | SGASMKLTTNGRFREHNAKF | 33 |
| P154. | NLIGDFEVAEKINAFRAKVH | 34 |
| P199. | GHSVLTAKGMALFGEGKAEF | 35 |
| P222. | FKSSVITLNTNAELFNQSDI | 36 |
| P240. | FPDLGQEVALNANTKNQKIR | 37 |

Category G. No level of IgG or IgM antibodies

| | | |
|---|---|---|
| P2 | ATRFKHLRKYTYNYEAESSS | 38 |

As shown in Table 1 above, the peptides could be grouped into seven categories with common characteristics:
Category A: Fragments that produce high levels of IgG antibodies to MDA modified peptides (n=3).
Category B: Fragments that produce high levels of IgM antibodies, but no difference between native and MDA-modified peptides (n=9).
Category C: Fragments that produce high levels of IgG antibodies, but no 10 difference between native and MDA-modified peptides (n=2).
Category D: Fragments that produce high levels of IgG antibodies to MDA modified peptides and at least twice as much antibodies in the NHP-pool as compared to the AHP-pool (n=5).
Category E: Fragments that produce high levels of IgM antibodies to MDA modified peptides and at least twice as much antibodies in the NHP-pool as compared to the AHP-pool (n=11).
Category F: Fragments that produce high levels of IgG antibodies, but no difference between intact and MDA-modified peptides but at least twice as much antibodies in the AHP-pool as compared to the NHP-pool (n=7).
Category G: Fragments that produce no level of IgG or IgM antibodies.

In some embodiments, the anti-oxidized LDL antibody is capable of binding to (e.g., binds to) an epitope of oxidized ApoB-100 is selected from the group consisting of SEQ ID NO:13, SEQ ID NO:14, SEQ ID NO:31, SEQ ID NO:32, SEQ ID NO:33, SEQ ID NO:34, SEQ ID NO:35, SEQ ID NO:36, SEQ ID NO:37. In some embodiments, the anti-oxidized LDL antibody is capable of binding to an epitope of oxidized ApoB-100 is selected from the group consisting of SEQ ID NO: 13, SEQ ID NO:14, SEQ ID NO:16, and SEQ ID NO:32. In some embodiments, the anti-oxidized LDL antibody is capable of binding to SEQ ID NO:32 (IEIGLEGKGFEPTLEALFGK).

It is appreciated that the peptide fragments of ApoB-100 may be made using protein chemistry techniques for example using partial proteolysis (either exolytically or endolytically), or by de novo synthesis. Alternatively, the variants may be made by recombinant DNA technology. Suitable techniques for cloning, manipulation, modification and expression of nucleic acids, and purification of expressed proteins, are well known hi the art and are described for example in Sambrook et al (2001) “Molecular Cloning, a Laboratory Manual”, 3rd edition, Sambrook et al (eds), Cold Spring Harbor Laboratory Press, Cold Spring Harbor, N.Y., USA, incorporated herein by reference.

By “peptide” we include not only molecules in which amino acid residues are joined by peptide (—CO—NH—) linkages but also molecules in which the peptide bond is reversed. Such retro-inverso peptidomimetics may be made using methods known in the art, for example such as those described in Meziere et al., J. Immunol. 159(7):3230-7 (1997). This approach involves making pseudopeptides containing changes involving the backbone, and not the orientation of side chains. At least for MHC class II and T helper cell responses, these pseudopeptides were shown to be useful. Retro-inverse peptides, which contain NH—CO bonds instead of CO—NH peptide bonds, are much more resistant to proteolysis. Similarly, the peptide bond may be dispensed with altogether provided that an appropriate linker moiety which retains the spacing between the Cα atoms of the amino acid residues is used. In some embodiments, the linker moiety has substantially the same charge distribution and substantially the same planarity of a peptide bond. It is also appreciated that the peptide may conveniently be blocked at its N- or C-terminus so as to help reduce susceptibility to exoproteolytic digestion.

In some embodiments, the oxidized LDL antibody binds a fragment of a peptide epitope of ApoB-100 listed in Table 1 (SEQ ID NO:1-38). A “fragment of a peptide epitope of ApoB-100” listed in Table 1 (SEQ ID NO:1-38) consists of at least six consecutive amino acids of the given sequence in Table 1 (SEQ ID NO:1-38). In some embodiments, a fragment of a peptide epitope of ApoB-100 may include about any of 6, 7, 8, 9, 10, 11, 12, 13, 14, 15, 16, 17, 18, or 19 consecutive amino acids of the given sequence.

In some embodiment, the anti-oxidized LDL antibody is an antibody raised against an oxidized epitope of LDL, such as those listed in Table 1 (SEQ ID NO:1-38). Peptides may be oxidized by exposure to a variety of agents such as iron, oxygen, copper, myeloperoxidase, phospholipase, hypochlorous acid, or by malone dealdehyde (MDA) modification, to mimic the different modifications of the amino acids that may occur during oxidation of LDL. Alternatively, other methods known in the art may be employed to oxidize the epitopes of LDL.

The antibodies may be characterized in a number of ways which will be apparent to those skilled in the art. These include physical measurements of their concentration by techniques such as ELISA, and of the antibody purity by SDS-PAGE. In addition the efficacy of the polypeptides can be determined by detecting the binding of the molecule to oxidized LDL in solution or in a solid phase system such as ELISA, surface plasmon resonance (e.g., BIAcore) or immunofluorescence assays. In some embodiments, the anti-oxidized LDL antibody binds to oxidized LDL with great affinity than native and/or unoxidized LDL (i.e., selectively binds to an epitope of oxidized LDL). In some embodiments, the anti-oxidized LDL antibody binds the oxidized epitope of LDL with at least about any of 1.5, 2, 5, 10, or 50 times greater affinity than for unoxidized LDL. In some embodiments, the anti-oxidized LDL antibody molecule binds the oxidized epitope of LDL with at least about any of 100, 1,000, or 10,000 times greater affinity than for unoxidized LDL. Such binding may be determined by methods well known in the art, such as one of the Biacore® systems. In some embodiments, the anti-oxidized LDL antibody binds to an epitope of oxidized LDL, but does not bind to native/unoxidized LDL. Such oxidized and native LDL epitopes can be determined for example by methods described in WO 02/080954.

In some embodiments, the antibodies have an affinity for their target epitope of at least about any of 10⁻⁴ M, 10⁻⁶ M, or 10⁻⁸ M or higher.

In some embodiments, the anti-oxidized LDL antibody further reduces inflammation. In some embodiments, the inflammation is associated with diabetes. In some embodiments, the anti-oxidized LDL antibody further reduces levels of an inflammatory marker. In some embodiments, the inflammatory marker is selected from the group consisting of IL-1β, IL-15, IL-8, IL-6, MCP-1, EN-RAGE, and TNF-α. In some embodiments, the inflammatory marker is selected from the group consisting of IL-1β, IL-15, EN-RAGE, and TNF-α. In some embodiments, the inflammation or levels of an inflammatory marker are reduced compared to inflammation or levels of an inflammatory marker prior to treatment with the anti-oxidized LDL antibody.

### (i) Definitions

The term “antibody” herein is used in the broadest sense and specifically covers monoclonal antibodies, polyclonal antibodies, multispecific antibodies (e.g. bispecific antibodies) formed from at least two intact antibodies, and antibody fragments so long as they exhibit the desired biological activity. The term “immunoglobulin” (Ig) is used interchangeable with antibody herein.

Antibodies are naturally occurring immunoglobulin molecules which have varying structures, all based upon the immunoglobulin fold. For example, IgG antibodies have two ‘heavy’ chains and two ‘light’ chains that are disulphide-bonded to form a functional antibody. Each heavy and light chain itself comprises a “constant” (C) and a “variable” (V) region. The V regions determine the antigen binding specificity of the antibody, whilst the C regions provide structural support and function in non-antigen-specific interactions with immune effectors. The antigen binding specificity of an antibody or antigen-binding fragment of an antibody is the ability of an antibody to specifically bind to a particular antigen.

The antigen binding specificity of an antibody is determined by the structural characteristics of the V region. The variability is not evenly distributed across the 110-amino acid span of the variable domains. Instead, the V regions consist of relatively invariant stretches called framework regions (FRs) of 15-30 amino acids separated by shorter regions of extreme variability called “hypervariable regions” that are each 9-12 amino acids long. The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

Each V region typically comprises three complementarity determining regions (“CDRs”, each of which contains a “hypervariable loop”), and four framework regions. An antibody binding site, the minimal structural unit required to bind with substantial affinity to a particular desired antigen, will therefore typically include the three CDRs, and at least three, preferably four, framework regions interspersed there between to hold and present the CDRs in the appropriate conformation. Classical four chain antibodies have antigen binding sites which are defined by V_{H} and V_{L} domains in cooperation. Certain antibodies, such as camel and shark antibodies, lack light chains and rely on binding sites formed by heavy chains only. Single domain engineered immunoglobulins can be prepared in which the binding sites are formed by heavy chains or light chains alone, in absence of cooperation between V_{H} and V_{L}.

Throughout the present specification and claims, unless otherwise indicated, the numbering of the residues in the constant domains of an immunoglobulin heavy chain is that of the EU index as in Kabat et al., Sequences of Proteins of Immunological Interest, 5th Ed. Public Health Service, National Institutes of Health, Bethesda, Md. (1991), expressly incorporated herein by reference. The “EU index as in Kabat” refers to the residue numbering of the human IgG1 EU antibody. The residues in the V region are numbered according to Kabat numbering unless sequential or other numbering system is specifically indicated.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody that are responsible for antigen binding. The hypervariable region may comprise amino acid residues from a “complementarity determining region” or “CDR” (e.g., around about residues 24-34 (L1), 50-56 (L2) and 89-97 (L3) in the V_{L}, and around about 31-35B (H1), 50-65 (H2) and 95-102 (H3) in the V_{H} (Kabat et al., Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the V_{L}, and 26-32 (H1), 52A-55 (H2) and 96-101 (H3) in the V_{H} (Chothia and Lesk J. Mol. Biol. 196:901-917 (1987)).

“Framework” or “FR” residues are those variable domain residues other than the hypervariable region residues as herein defined.

“Antibody fragments” comprise a portion of an intact antibody, preferably comprising the antigen binding region thereof. Examples of antibody fragments include Fab, Fab′, F(ab′)₂, and Fv fragments; diabodies; linear antibodies; single-chain antibody molecules; and multispecific antibodies formed from antibody fragments.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-binding sites and is still capable of cross-linking antigen.

“Fv” is the minimum antibody fragment that contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the V_{H}-V_{L} dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear at least one free thiol group. F(ab′)₂ antibody fragments originally were produced as pairs of Fab′ fragments that have hinge cysteines between them. Other chemical couplings of antibody fragments are also known.

The “light chains” of antibodies (immunoglobulins) from any vertebrate species can be assigned to one of two clearly distinct types, called kappa (κ) and lambda (λ), based on the amino acid sequences of their constant domains.

Depending on the amino acid sequence of the constant domain of their heavy chains, antibodies can be assigned to different classes. There are five major classes of intact antibodies: IgA, IgD, IgE, IgG, and IgM, and several of these may be further divided into subclasses (isotypes), e.g., IgG1, IgG2, IgG3, IgG4, IgA, and IgA2. The heavy chain constant domains that correspond to the different classes of antibodies are called α, δ, ε, γ, and μ, respectively. The subunit structures and three-dimensional configurations of different classes of immunoglobulins are well known.

“Single-chain Fv” or “scFv” antibody fragments comprise the V_{H} and V_{L} domains of antibody, wherein these domains are present in a single polypeptide chain. In some embodiments, the Fv polypeptide further comprises a polypeptide linker between the V_{H} and V_{L} domains that enables the scFv to form the desired structure for antigen binding. For a review of scFv see Plückthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy chain variable domain (V_{H}) connected to a light chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

The term “monoclonal antibody” as used herein refers to an antibody obtained from a population of substantially homogeneous antibodies, i.e., the individual antibodies comprising the population are identical and/or bind the same epitope, except for possible variants that may arise during production of the monoclonal antibody, such variants generally being present in minor amounts. In contrast to polyclonal antibody preparations that typically include different antibodies directed against different determinants (epitopes), each monoclonal antibody is directed against a single determinant on the antigen. In addition to their specificity, the monoclonal antibodies are advantageous in that they are uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the methods provided herein may be made by the hybridoma method first described by Kohler et al., Nature 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature 352:624-628 (1991) and Marks et al., J. Mol. Biol. 222:581-597 (1991), for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA 81:6851-6855 (1984)) Chimeric antibodies of interest herein include “primatized” antibodies comprising variable domain antigen-binding sequences derived from a non-human primate (e.g. Old World Monkey, such as baboon, rhesus or cynomolgus monkey) and human constant region sequences (U.S. Pat. No. 5,693,780).

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence, except for FR substitution(s) as noted above. The humanized antibody optionally also will comprise at least a portion of an immunoglobulin constant region, typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

For the purposes herein, an “intact antibody” is one comprising heavy and light variable domains as well as an Fc region.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

A “naked antibody” is an antibody (as herein defined) that is not conjugated to a heterologous molecule, such as a cytotoxic moiety or radiolabel.

An “isolated” antibody is one that has been identified and separated and/or recovered from a component of its natural environment. Contaminant components of its natural environment are materials that would interfere with diagnostic or therapeutic uses for the antibody, and may include enzymes, hormones, and other proteinaceous or non-proteinaceous solutes. In some embodiments, the antibody will be purified (1) to greater than 95% by weight of antibody as determined by the Lowry method, and in some embodiments, more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, in some embodiments, silver stain. Isolated antibody includes the antibody in situ within recombinant cells since at least one component of the antibody's natural environment will not be present. Ordinarily, however, isolated antibody will be prepared by at least one purification step. The antibody or antibody fragment described herein may be isolated or purified to any degree.

“Purified” means that the antibody or antibody fragment has been increased in purity, such that it exists in a form that is more pure than it exists in its natural environment and/or when initially synthesized and/or amplified under laboratory conditions. Purity is a relative term and does not necessarily mean absolute purity.

In some embodiments, antibody “effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors.

“Antibody-dependent cell-mediated cytotoxicity” and “ADCC” refer to a cell-mediated reaction in which nonspecific cytotoxic cells that express Fc receptors (FcRs) (e.g. Natural Killer (NK) cells, neutrophils, and macrophages) recognize bound antibody on a target cell and subsequently cause lysis of the target cell. The primary cells for mediating ADCC, NK cells, express FcγRIII only, whereas monocytes express FcγRI, FcγRII and FcγRIII. FcR expression on hematopoietic cells in summarized is Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or U.S. Pat. No. 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al., PNAS (USA) 95:652-656 (1998).

“Human effector cells” are leukocytes that express one or more FcRs and perform effector functions. In some embodiments, the cells express at least FcγRIII and carry out ADCC effector function. Examples of human leukocytes that mediate ADCC include peripheral blood mononuclear cells (PBMC), natural killer (NK) cells, monocytes, cytotoxic T-cells and neutrophils; with PBMCs and NK cells being preferred.

The terms “Fc receptor” or “FcR” are used to describe a receptor that binds to the Fc region of an antibody. In some embodiments, the FcR is a native sequence human FcR. Moreover, a preferred FcR is one that binds an IgG antibody (a gamma receptor) and includes receptors of the FcγRI, FcγRII, and Fcγ RIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain Inhibiting receptor FcγRIIB contains an immunoreceptor tyrosine-based inhibition motif (ITIM) in its cytoplasmic domain. (see Daëron, Annu. Rev. Immunol. 15:203-234 (1997)). FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)).

“Complement dependent cytotoxicity” or “CDC” refers to the ability of a molecule to lyse a target in the presence of complement. The complement activation pathway is initiated by the binding of the first component of the complement system (C1q) to a molecule (e.g. an antibody) complexed with a cognate antigen. To assess complement activation, a CDC assay, e.g. as described in Gazzano-Santoro et al., J. Immunol. Methods 202:163 (1996), may be performed.

### (ii) Polyclonal Antibodies

In some embodiments, the anti-oxidized LDL antibodies are polyclonal antibodies. Polyclonal antibodies are preferably raised in animals by multiple subcutaneous (sc) or intraperitoneal (ip) injections of the relevant antigen and an adjuvant (for examples of relevant antigen, see PCT/GB2006/000987, which is incorporated by reference in its entirety). It may be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are different alkyl groups.

Animals are immunized against the antigen, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later the animals are boosted with ⅕ to 1/10 the original amount of peptide or conjugate in Freund's complete adjuvant by subcutaneous injection at multiple sites. Seven to 14 days later the animals are bled and the serum is assayed for antibody titer. Animals are boosted until the titer plateaus. In some embodiments, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

(iii) Monoclonal Antibodies

In some embodiments, the anti-oxidized LDL antibodies are monoclonal antibodies. Monoclonal antibodies are obtained from a population of substantially homogeneous antibodies, i.e., the individual antibodies comprising the population are identical and/or bind the same epitope except for possible variants that arise during production of the monoclonal antibody, such variants generally being present in minor amounts. Thus, the modifier “monoclonal” indicates the character of the antibody as not being a mixture of discrete or polyclonal antibodies.

For example, the monoclonal antibodies may be made using the hybridoma method first described by Kohler et al., Nature 256:495 (1975), or may be made by recombinant DNA methods (U.S. Pat. No. 4,816,567).

In the hybridoma method, a mouse or other appropriate host animal, such as a hamster, is immunized as herein described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

In some embodiments, the myeloma cells are those that fuse efficiently, support stable high-level production of antibody by the selected antibody-producing cells, and are sensitive to a medium such as HAT medium. Among these, in some embodiments, the myeloma cell lines are murine myeloma lines, such as those derived from MOPC-21 and MPC-11 mouse tumors available from the Salk Institute Cell Distribution Center, San Diego, Calif. USA, and SP-2 or X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. In some embodiments, the binding specificity of monoclonal antibodies produced by hybridoma cells is determined by immunoprecipitation or by an in vitro binding assay, such as radioimmunoassay (RIA) or enzyme-linked immunoabsorbent assay (ELISA).

The binding affinity of the monoclonal antibody can, for example, be determined by the Scatchard analysis of Munson et al., Anal. Biochem. 107:220 (1980).

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

DNA encoding the monoclonal antibodies is readily isolated and sequenced using conventional procedures (e.g., by using oligonucleotide probes that are capable of binding specifically to genes encoding the heavy and light chains of murine antibodies). In some embodiments, the hybridoma cells serve as a source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce immunoglobulin protein, to obtain the synthesis of monoclonal antibodies in the recombinant host cells. Review articles on recombinant expression in bacteria of DNA encoding the antibody include Skerra et al., Curr. Opinion in Immunol. 5:256-262 (1993) and Plückthun, Immunol. Revs., 130:151-188 (1992).

In a further embodiment, antibodies or antibody fragments can be isolated from antibody phage libraries generated using the techniques described in McCafferty et al., Nature 348:552-554 (1990). Clackson et al., Nature 352:624-628 (1991) and Marks et al., J. Mol. Biol. 222:581-597 (1991) describe the isolation of murine and human antibodies, respectively, using phage libraries. Subsequent publications describe the production of high affinity (nM range) human antibodies by chain shuffling (Marks et al., Bio/Technology 10:779-783 (1992)), as well as combinatorial infection and in vivo recombination as a strategy for constructing very large phage libraries (Waterhouse et al., Nuc. Acids. Res. 21:2265-2266 (1993)). Thus, these techniques are viable alternatives to traditional monoclonal antibody hybridoma techniques for isolation of monoclonal antibodies.

The DNA also may be modified, for example, by substituting the coding sequence for human heavy- and light chain constant domains in place of the homologous murine sequences (U.S. Pat. No. 4,816,567; Morrison, et al., Proc. Natl. Acad. Sci. USA 81:6851 (1984)), or by covalently joining to the immunoglobulin coding sequence all or part of the coding sequence for a non-immunoglobulin polypeptide.

Typically such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

In some embodiments, the monoclonal antibody comprises one or more (at least one, two, three, four, five, or six) CDRs from IEIA8, IEI-G8, IEI-D8, KTT-D6, 1-B12, 1-C07, 1-C12, 1-G10, 2-F07, 2-F09, 4-A02, IEI-E3, 2D03, LDO-D4, and/or KTT-B8. In some embodiments, the monoclonal antibody comprises one or more (at least one, two, three, four, five, or six) CDRs from IEI-G8, IEI-D8, IEI-E3, 2D03, LDO-D4, and/or KTT-B8.

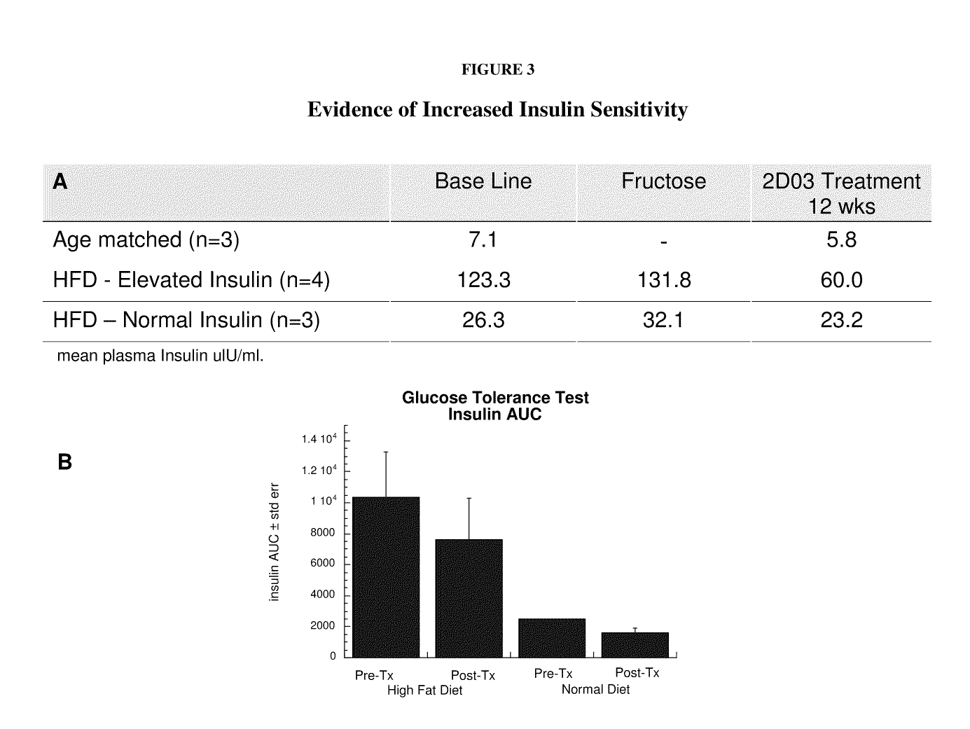

In some embodiments, the monoclonal antibody comprises one or more (at least one, two, three, four, five, or six) CDRs from a sequence selected from the group consisting of SEQ ID NO:64, SEQ ID NO:68, SEQ ID NO:72, SEQ ID NO:76, SEQ ID NO:80, SEQ ID NO:84, SEQ ID NO:88, SEQ ID NO:92, SEQ ID NO:96, SEQ ID NO:100, SEQ ID NO:104, SEQ ID NO:108, SEQ ID NO:112, SEQ ID NO:116, SEQ ID NO:120, SEQ ID NO:124, SEQ ID NO:66, SEQ ID NO:70, SEQ ID NO:74, SEQ ID NO:78, SEQ ID NO:82, SEQ ID NO:86, SEQ ID NO:90, SEQ ID NO:94, SEQ ID NO:98, SEQ ID NO:102, SEQ ID NO:106, SEQ ID NO:110, SEQ ID NO:114, SEQ ID NO:118, SEQ ID NO:122, and SEQ ID NO:126. In some embodiments, the monoclonal antibody comprises one or more (at least one, two, three, four, five, or six) CDRs derived from one or more V_{H} and/or V_{L} sequences of the antibodies in FIG. 3 of WO 2004/030607, which is incorporated by reference in its entirety. In some embodiments, the monoclonal antibody comprises one or more (at least one, two, three, four, five, or six) CDRs of the antibodies of Table 2 of WO 2007/025781, which is incorporated by reference in its entirety.

In some embodiments, the monoclonal antibody comprises one or more (at least one, two, three, four, five, or six) CDRs selected from the group consisting of CSGSNTNIGKNYVS (SEQ ID NO:39), ANSNRPS (SEQ ID NO:40), CASWDASLNGWV (SEQ ID NO:41), FSNAWMSWVRQAPG (SEQ ID NO:42), SSISVGGHRTYYADSVKGR, (SEQ ID NO:43), and ARIRVGPSGGAFDY (SEQ ID NO:44). In some embodiments, the monoclonal antibody comprises a light chain variable domain comprising (i) CDR-L1 comprising sequence CSGSNTNIGKNYVS (SEQ ID NO:39); (ii) CDR-L2 comprising sequence ANSNRPS (SEQ ID NO:40); and (iii) CDR-L3 comprising sequence CASWDASLNGWV (SEQ ID NO:41). In some embodiments, the monoclonal antibody comprises a heavy chain variable domain comprising (i) CDR-H1 comprising sequence FSNAWMSWVRQAPG (SEQ ID NO:42); (ii) CDR-H2 comprising sequence SSISVGGHRTYYADSVKGR (SEQ ID NO:43); and (iii) CDR-H3 comprising sequence ARIRVGPSGGAFDY (SEQ ID NO:44). In some embodiments, the monoclonal antibody comprises a light chain variable domain comprising (i) CDR-L1 comprising sequence CSGSNTNIGKNYVS (SEQ ID NO:39); (ii) CDR-L2 comprising sequence ANSNRPS (SEQ ID NO:40); and (iii) CDR-L3 comprising sequence CASWDASLNGWV (SEQ ID NO:41) and a heavy chain variable domain comprising (i) CDR-H1 comprising sequence FSNAWMSWVRQAPG (SEQ ID NO:42); (ii) CDR-H2 comprising sequence SSISVGGHRTYYADSVKGR (SEQ ID NO:43); and (iii) CDR-H3 comprising sequence ARIRVGPSGGAFDY (SEQ ID NO:44).

In some embodiments, the monoclonal antibody comprises one or more (at least one, two, three, four, five, or six) CDRs selected from the group consisting of CSGSSSNIGNNAVN (SEQ ID NO:45), GNDRRPS (SEQ ID NO:46), CQTWGTGRGV (SEQ ID NO:47), FSDYYMSWVRQAPG (SEQ ID NO:48), SGVSWNGSRTHYADSVKGR (SEQ ID NO:49), and ARAARYSYYYYGMDV (SEQ ID NO:50). In some embodiments, the monoclonal antibody comprises a light chain variable domain comprising (i) CDR-L1 comprising sequence CSGSSSNIGNNAVN (SEQ ID NO:45); (ii) CDR-L2 comprising sequence GNDRRPS (SEQ ID NO:46); and (iii) CDR-L3 comprising sequence CQTWGTGRGV (SEQ ID NO:47). In some embodiments, the monoclonal antibody comprises a heavy chain variable domain comprising (i) CDR-H1 comprising sequence FSDYYMSWVRQAPG (SEQ ID NO:48); (ii) CDR-H2 comprising sequence SGVSWNGSRTHYADSVKGR (SEQ ID NO:49); and (iii) CDR-H3 comprising sequence ARAARYSYYYYGMDV (SEQ ID NO:50). In some embodiments, the monoclonal antibody comprises a light chain variable domain comprising (i) CDR-L1 comprising sequence CSGSSSNIGNNAVN (SEQ ID NO:45); (ii) CDR-L2 comprising sequence GNDRRPS (SEQ ID NO:46); and (iii) CDR-L3 comprising sequence CQTWGTGRGV (SEQ ID NO:47) and a heavy chain variable domain comprising (i) CDR-H1 comprising sequence FSDYYMSWVRQAPG (SEQ ID NO:48); (ii) CDR-H2 comprising sequence SGVSWNGSRTHYADSVKGR (SEQ ID NO:49); and (iii) CDR-H3 comprising sequence ARAARYSYYYYGMDV (SEQ ID NO:50).

In some embodiments, the monoclonal antibody comprises one or more (at least one, two, three, four, five, or six) CDRs selected from the group consisting of CSGSSSSIGNNFVS (SEQ ID NO:51), DNNKRPS (SEQ ID NO:52), CAAWDDSLNGWV (SEQ ID NO:53), FSNAWMSWVRQAPG (SEQ ID NO:54), SSISTSSNYIYYADSVKGR (SEQ ID NO:55), and ARVKKYSSGWYSNYAFDI (SEQ ID NO:56). In some embodiments, the monoclonal antibody comprises a light chain variable domain comprising (i) CDR-L1 comprising sequence CSGSSSSIGNNFVS (SEQ ID NO:51); (ii) CDR-L2 comprising sequence DNNKRPS (SEQ ID NO:52); and (iii) CDR-L3 comprising sequence CAAWDDSLNGWV (SEQ ID NO:53). In some embodiments, the monoclonal antibody comprises a heavy chain variable domain comprising (i) CDR-H1 comprising sequence FSNAWMSWVRQAPG (SEQ ID NO:54); (ii) CDR-H2 comprising sequence SSISTSSNYIYYADSVKGR (SEQ ID NO:55); and (iii) CDR-H3 comprising sequence ARVKKYSSGWYSNYAFDI (SEQ ID NO:56). In some embodiments, the monoclonal antibody comprises a light chain variable domain comprising (i) CDR-L1 comprising sequence CSGSSSSIGNNFVS (SEQ ID NO:51); (ii) CDR-L2 comprising sequence DNNKRPS (SEQ ID NO:52); and (iii) CDR-L3 comprising sequence CAAWDDSLNGWV (SEQ ID NO:53) and a heavy chain variable domain comprising (i) CDR-H1 comprising sequence FSNAWMSWVRQAPG (SEQ ID NO:54); (ii) CDR-H2 comprising sequence SSISTSSNYIYYADSVKGR (SEQ ID NO:55); and (iii) CDR-H3 comprising sequence ARVKKYSSGWYSNYAFDI (SEQ ID NO:56).

In some embodiments, the monoclonal antibody comprises one or more (at least one, two, three, four, five, or six) CDRs selected from the group consisting of CSGSSSNIGGESVS (SEQ ID NO:57), SNNQRPS (SEQ ID NO:58), CAAWDDSLNGWV (SEQ ID NO:59), FSSYAMSWVRQAPG (SEQ ID NO:60), SSISSSGRFIYYADSMKGR (SEQ ID NO:61), and TRLRRGSYFWAFDI (SEQ ID NO:62). In some embodiments, the monoclonal antibody comprises a light chain variable domain comprising (i) CDR-L1 comprising sequence CSGSSSNIGGESVS (SEQ ID NO:57); (ii) CDR-L2 comprising sequence SNNQRPS (SEQ ID NO:58); and (iii) CDR-L3 comprising sequence CAAWDDSLNGWV (SEQ ID NO:59). In some embodiments, the monoclonal antibody comprises a heavy chain variable domain comprising (i) CDR-H1 comprising sequence FSSYAMSWVRQAPG (SEQ ID NO:60); (ii) CDR-H2 comprising sequence SSISSSGRFIYYADSMKGR (SEQ ID NO:61); and (iii) CDR-H3 comprising sequence TRLRRGSYFWAFDI (SEQ ID NO:62). In some embodiments, the monoclonal antibody comprises a light chain variable domain comprising (i) CDR-L1 comprising sequence CSGSSSNIGGESVS (SEQ ID NO:57); (ii) CDR-L2 comprising sequence SNNQRPS (SEQ ID NO:58); and (iii) CDR-L3 comprising sequence CAAWDDSLNGWV (SEQ ID NO:59) and a heavy chain variable domain comprising (i) CDR-H1 comprising sequence FSSYAMSWVRQAPG (SEQ ID NO:60); (ii) CDR-H2 comprising sequence SSISSSGRFIYYADSMKGR (SEQ ID NO:61); and (iii) CDR-H3 comprising sequence TRLRRGSYFWAFDI (SEQ ID NO:62).

In some embodiments, the monoclonal antibody comprises one or more (at least one, two, three, four, five, or six) CDRs selected from the group consisting of CSGSSSNIGNNYVS (SEQ ID NO:127), SNNQRPS (SEQ ID NO:128), CAAWDDSLSHWL (SEQ ID NO:129), FSNAWMSWVRQVPG (SEQ ID NO:130), STLGGSGGGSTYYADSVKGR (SEQ ID NO:131), and AKLGGRSRYGRWPRQFDY (SEQ ID NO:132). In some embodiments, the monoclonal antibody comprises a light chain variable domain comprising (i) CDR-L1 comprising sequence CSGSSSNIGNNYVS (SEQ ID NO:127); (ii) CDR-L2 comprising sequence SNNQRPS (SEQ ID NO:128); and (iii) CDR-L3 comprising sequence CAAWDDSLSHWL (SEQ ID NO:129). In some embodiments, the monoclonal antibody comprises a heavy chain variable domain comprising (i) CDR-H1 comprising sequence FSNAWMSWVRQVPG (SEQ ID NO:130); (ii) CDR-H2 comprising sequence STLGGSGGGSTYYADSVKGR (SEQ ID NO:131); and (iii) CDR-H3 comprising sequence AKLGGRSRYGRWPRQFDY (SEQ ID NO:132). In some embodiments, the monoclonal antibody comprises a light chain variable domain comprising (i) CDR-L1 comprising sequence CSGSSSNIGNNYVS (SEQ ID NO:127); (ii) CDR-L2 comprising sequence SNNQRPS (SEQ ID NO:128); and (iii) CDR-L3 comprising sequence CAAWDDSLSHWL (SEQ ID NO:129) and a heavy chain variable domain comprising (i) CDR-H1 comprising sequence FSNAWMSWVRQVPG (SEQ ID NO:130); (ii) CDR-H2 comprising sequence STLGGSGGGSTYYADSVKGR (SEQ ID NO:131); and (iii) CDR-H3 comprising sequence AKLGGRSRYGRWPRQFDY (SEQ ID NO:132.

In some embodiments, the monoclonal antibody comprises one or more (at least one, two, three, four, five, or six) CDRs selected from the group consisting of CSGSSSNIGSNYVS (SEQ ID NO:133), GNYNRPS (SEQ ID NO:134), CAAWDDSLSGWV (SEQ ID NO:135), FSSYWMSWVRQAPG (SEQ ID NO:136), SSISGSGRRTYYADSVQGR (SEQ ID NO:137), and ARLVSYGSGSFGFDY (SEQ ID NO:138). In some embodiments, the monoclonal antibody comprises a light chain variable domain comprising (i) CDR-L1 comprising sequence CSGSSSNIGSNYVS (SEQ ID NO:133); (ii) CDR-L2 comprising sequence GNYNRPS (SEQ ID NO:134); and (iii) CDR-L3 comprising sequence CAAWDDSLSGWV (SEQ ID NO:135). In some embodiments, the monoclonal antibody comprises a heavy chain variable domain comprising (i) CDR-H1 comprising sequence FSSYWMSWVRQAPG (SEQ ID NO:136); (ii) CDR-H2 comprising sequence SSISGSGRRTYYADSVQGR (SEQ ID NO:137); and (iii) CDR-H3 comprising sequence ARLVSYGSGSFGFDY (SEQ ID NO:138). In some embodiments, the monoclonal antibody comprises a light chain variable domain comprising (i) CDR-L1 comprising sequence CSGSSSNIGSNYVS (SEQ ID NO:133); (ii) CDR-L2 comprising sequence GNYNRPS (SEQ ID NO:134); and (iii) CDR-L3 comprising sequence CAAWDDSLSGWV (SEQ ID NO:135) and a heavy chain variable domain comprising (i) CDR-H1 comprising sequence FSSYWMSWVRQAPG (SEQ ID NO:136); (ii) CDR-H2 comprising sequence SSISGSGRRTYYADSVQGR (SEQ ID NO:137); and (iii) CDR-H3 comprising sequence ARLVSYGSGSFGFDY (SEQ ID NO:138).

In some embodiments, the monoclonal antibody comprises one or more (at least one, two, three, four, five, or six) CDRs selected from the group consisting of CSGRSSNIGNSYVS (SEQ ID NO:139), RNNQRPS (SEQ ID NO:140), CAGWDDTLRAWV (SEQ ID NO:141), FRDYYVSWIRQAPG (SEQ ID NO:142), SSISGSGGRTYYADSVEGR (SEQ ID NO:143), and ARVSALRRPMTTVTTYWFDP (SEQ ID NO:144). In some embodiments, the monoclonal antibody comprises a light chain variable domain comprising (i) CDR-L1 comprising sequence CSGRSSNIGNSYVS (SEQ ID NO:139); (ii) CDR-L2 comprising sequence RNNQRPS (SEQ ID NO:140); and (iii) CDR-L3 comprising sequence CAGWDDTLRAWV (SEQ ID NO:141). In some embodiments, the monoclonal antibody comprises a heavy chain variable domain comprising (i) CDR-H1 comprising sequence FRDYYVSWIRQAPG (SEQ ID NO:142); (ii) CDR-H2 comprising sequence SSISGSGGRTYYADSVEGR (SEQ ID NO:143); and (iii) CDR-H3 comprising sequence ARVSALRRPMTTVTTYWFDP (SEQ ID NO:144). In some embodiments, the monoclonal antibody comprises a light chain variable domain comprising (i) CDR-L1 comprising sequence CSGRSSNIGNSYVS (SEQ ID NO:139); (ii) CDR-L2 comprising sequence RNNQRPS (SEQ ID NO:140); and (iii) CDR-L3 comprising sequence CAGWDDTLRAWV (SEQ ID NO:141) and a heavy chain variable domain comprising (i) CDR-H1 comprising sequence FRDYYVSWIRQAPG (SEQ ID NO:142); (ii) CDR-H2 comprising sequence SSISGSGGRTYYADSVEGR (SEQ ID NO:143); and (iii) CDR-H3 comprising sequence ARVSALRRPMTTVTTYWFDP (SEQ ID NO:144).

### (iv) Humanized Antibodies

In some embodiments, the anti-oxidized LDL antibodies are humanized antibodies. Methods for humanizing non-human antibodies have been described in the art. In some embodiments, a humanized antibody has one or more amino acid residues introduced into it from a source that is non-human. These non-human amino acid residues are often referred to as “import” residues, which are typically taken from an “import” variable domain Humanization can be essentially performed following the method of Winter and co-workers (Jones et al., Nature 321:522-525 (1986); Riechmann et al., Nature 332:323-327 (1988); Verhoeyen et al., Science 239:1534-1536 (1988)), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence that is closest to that of the rodent is then accepted as the human framework region (FR) for the humanized antibody (Sims et al., J. Immunol. 151:2296 (1993); Chothia et al., J. Mol. Biol. 196:901 (1987)). Another method uses a particular framework region derived from the consensus sequence of all human antibodies of a particular subgroup of light or heavy chain variable regions. The same framework may be used for several different humanized antibodies (Carter et al., Proc. Natl. Acad. Sci. USA 89:4285 (1992); Presta et al., J. Immunol. 151:2623 (1993)).

It is further important that antibodies be humanized with retention of high affinity for the antigen and other favorable biological properties. To achieve this goal, in some embodiments of the methods, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available that illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

In some embodiments, the humanized antibody comprises one or more (at least one, two, three, four, five, or six) CDRs from IEIA8, IEI-G8, IEI-D8, KTT-D6, 1-B12, 1-C07, 1-C12, 1-G10, 2-F07, 2-F09, 4-A02, IEI-E3, 2D03, LDO-D4, and/or KTT-B8. In some embodiments, the humanized antibody comprises one or more (at least one, two, three, four, five, or six) CDRs from IEI-G8, IEI-D8, IEI-E3, 2D03, LDO-D4, and/or KTT-B8.

In some embodiments, the humanized antibody comprises one or more (at least one, two, three, four, five, or six) CDRs from a sequence selected from the group consisting of SEQ ID NO:64, SEQ ID NO:68, SEQ ID NO:72, SEQ ID NO:76, SEQ ID NO:80, SEQ ID NO:84, SEQ ID NO:88, SEQ ID NO:92, SEQ ID NO:96, SEQ ID NO:100, SEQ ID NO:104, SEQ ID NO:108, SEQ ID NO:112, SEQ ID NO:116, SEQ ID NO:120, SEQ ID NO:124, SEQ ID NO:66, SEQ ID NO:70, SEQ ID NO:74, SEQ ID NO:78, SEQ ID NO:82, SEQ ID NO:86, SEQ ID NO:90, SEQ ID NO:94, SEQ ID NO:98, SEQ ID NO:102, SEQ ID NO:106, SEQ ID NO:110, SEQ ID NO:114, SEQ ID NO:118, SEQ ID NO:122, and SEQ ID NO:126. In some embodiments, the humanized antibody comprises one or more (at least one, two, three, four, five, or six) CDRs derived from one or more V_{H} and/or V_{L} sequences of the antibodies in FIG. 3 of WO 2004/030607, which is incorporated by reference in its entirety. In some embodiments, the humanized antibody comprises one or more (at least one, two, three, four, five, or six) CDRs of the antibodies of Table 2 of WO 2007/025781, which is incorporated by reference in its entirety.

In some embodiments, the humanized antibody comprises one or more (at least one, two, three, four, five, or six) CDRs selected from the group consisting of CSGSNTNIGKNYVS (SEQ ID NO:39), ANSNRPS (SEQ ID NO:40), CASWDASLNGWV (SEQ ID NO:41), FSNAWMSWVRQAPG (SEQ ID NO:42), SSISVGGHRTYYADSVKGR, (SEQ ID NO:43), and ARIRVGPSGGAFDY (SEQ ID NO:44). In some embodiments, the humanized antibody comprises a light chain variable domain comprising (i) CDR-L1 comprising sequence CSGSNTNIGKNYVS (SEQ ID NO:39); (ii) CDR-L2 comprising sequence ANSNRPS (SEQ ID NO:40); and (iii) CDR-L3 comprising sequence CASWDASLNGWV (SEQ ID NO:41). In some embodiments, the humanized antibody comprises a heavy chain variable domain comprising (i) CDR-H1 comprising sequence FSNAWMSWVRQAPG (SEQ ID NO:42); (ii) CDR-H2 comprising sequence SSISVGGHRTYYADSVKGR (SEQ ID NO:43); and (iii) CDR-H3 comprising sequence ARIRVGPSGGAFDY (SEQ ID NO:44). In some embodiments, the humanized antibody comprises a light chain variable domain comprising (i) CDR-L1 comprising sequence CSGSNTNIGKNYVS (SEQ ID NO:39); (ii) CDR-L2 comprising sequence ANSNRPS (SEQ ID NO:40); and (iii) CDR-L3 comprising sequence CASWDASLNGWV (SEQ ID NO:41) and a heavy chain variable domain comprising (i) CDR-H1 comprising sequence FSNAWMSWVRQAPG (SEQ ID NO:42); (ii) CDR-H2 comprising sequence SSISVGGHRTYYADSVKGR (SEQ ID NO:43); and (iii) CDR-H3 comprising sequence ARIRVGPSGGAFDY (SEQ ID NO:44).

In some embodiments, the humanized antibody comprises one or more (at least one, two, three, four, five, or six) CDRs selected from the group consisting of CSGSSSNIGNNAVN (SEQ ID NO:45), GNDRRPS (SEQ ID NO:46), CQTWGTGRGV (SEQ ID NO:47), FSDYYMSWVRQAPG (SEQ ID NO:48), SGVSWNGSRTHYADSVKGR (SEQ ID NO:49), and ARAARYSYYYYGMDV (SEQ ID NO:50). In some embodiments, the humanized antibody comprises a light chain variable domain comprising (i) CDR-L1 comprising sequence CSGSSSNIGNNAVN (SEQ ID NO:45); (ii) CDR-L2 comprising sequence GNDRRPS (SEQ ID NO:46); and (iii) CDR-L3 comprising sequence CQTWGTGRGV (SEQ ID NO:47). In some embodiments, the humanized antibody comprises a heavy chain variable domain comprising (i) CDR-H1 comprising sequence FSDYYMSWVRQAPG (SEQ ID NO:48); (ii) CDR-H2 comprising sequence SGVSWNGSRTHYADSVKGR (SEQ ID NO:49); and (iii) CDR-H3 comprising sequence ARAARYSYYYYGMDV (SEQ ID NO:50). In some embodiments, the humanized antibody comprises a light chain variable domain comprising (i) CDR-L1 comprising sequence CSGSSSNIGNNAVN (SEQ ID NO:45); (ii) CDR-L2 comprising sequence GNDRRPS (SEQ ID NO:46); and (iii) CDR-L3 comprising sequence CQTWGTGRGV (SEQ ID NO:47) and a heavy chain variable domain comprising (i) CDR-H1 comprising sequence FSDYYMSWVRQAPG (SEQ ID NO:48); (ii) CDR-H2 comprising sequence SGVSWNGSRTHYADSVKGR (SEQ ID NO:49); and (iii) CDR-H3 comprising sequence ARAARYSYYYYGMDV (SEQ ID NO:50).

In some embodiments, the humanized antibody comprises one or more (at least one, two, three, four, five, or six) CDRs selected from the group consisting of CSGSSSSIGNNFVS (SEQ ID NO:51), DNNKRPS (SEQ ID NO:52), CAAWDDSLNGWV (SEQ ID NO:53), FSNAWMSWVRQAPG (SEQ ID NO:54), SSISTSSNYIYYADSVKGR (SEQ ID NO:55), and ARVKKYSSGWYSNYAFDI (SEQ ID NO:56). In some embodiments, the humanized antibody comprises a light chain variable domain comprising (i) CDR-L1 comprising sequence CSGSSSSIGNNFVS (SEQ ID NO:51); (ii) CDR-L2 comprising sequence DNNKRPS (SEQ ID NO:52); and (iii) CDR-L3 comprising sequence CAAWDDSLNGWV (SEQ ID NO:53). In some embodiments, the humanized antibody comprises a heavy chain variable domain comprising (i) CDR-H1 comprising sequence FSNAWMSWVRQAPG (SEQ ID NO:54); (ii) CDR-H2 comprising sequence SSISTSSNYIYYADSVKGR (SEQ ID NO:55); and (iii) CDR-H3 comprising sequence ARVKKYSSGWYSNYAFDI (SEQ ID NO:56). In some embodiments, the humanized antibody comprises a light chain variable domain comprising (i) CDR-L1 comprising sequence CSGSSSSIGNNFVS (SEQ ID NO:51); (ii) CDR-L2 comprising sequence DNNKRPS (SEQ ID NO:52); and (iii) CDR-L3 comprising sequence CAAWDDSLNGWV (SEQ ID NO:53) and a heavy chain variable domain comprising (i) CDR-H1 comprising sequence FSNAWMSWVRQAPG (SEQ ID NO:54); (ii) CDR-H2 comprising sequence SSISTSSNYIYYADSVKGR (SEQ ID NO:55); and (iii) CDR-H3 comprising sequence ARVKKYSSGWYSNYAFDI (SEQ ID NO:56).

In some embodiments, the humanized antibody comprises one or more (at least one, two, three, four, five, or six) CDRs selected from the group consisting of CSGSSSNIGGESVS (SEQ ID NO:57), SNNQRPS (SEQ ID NO:58), CAAWDDSLNGWV (SEQ ID NO:59), FSSYAMSWVRQAPG (SEQ ID NO:60), SSISSSGRFIYYADSMKGR (SEQ ID NO:61), and TRLRRGSYFWAFDI (SEQ ID NO:62). In some embodiments, the humanized antibody comprises a light chain variable domain comprising (i) CDR-L1 comprising sequence CSGSSSNIGGESVS (SEQ ID NO:57); (ii) CDR-L2 comprising sequence SNNQRPS (SEQ ID NO:58); and (iii) CDR-L3 comprising sequence CAAWDDSLNGWV (SEQ ID NO:59). In some embodiments, the humanized antibody comprises a heavy chain variable domain comprising (i) CDR-H1 comprising sequence FSSYAMSWVRQAPG (SEQ ID NO:60); (ii) CDR-H2 comprising sequence SSISSSGRFIYYADSMKGR (SEQ ID NO:61); and (iii) CDR-H3 comprising sequence TRLRRGSYFWAFDI (SEQ ID NO:62). In some embodiments, the humanized antibody comprises a light chain variable domain comprising (i) CDR-L1 comprising sequence CSGSSSNIGGESVS (SEQ ID NO:57); (ii) CDR-L2 comprising sequence SNNQRPS (SEQ ID NO:58); and (iii) CDR-L3 comprising sequence CAAWDDSLNGWV (SEQ ID NO:59) and a heavy chain variable domain comprising (i) CDR-H1 comprising sequence FSSYAMSWVRQAPG (SEQ ID NO:60); (ii) CDR-H2 comprising sequence SSISSSGRFIYYADSMKGR (SEQ ID NO:61); and (iii) CDR-H3 comprising sequence TRLRRGSYFWAFDI (SEQ ID NO:62).

In some embodiments, the humanized antibody comprises one or more (at least one, two, three, four, five, or six) CDRs selected from the group consisting of CSGSSSNIGNNYVS (SEQ ID NO:127), SNNQRPS (SEQ ID NO:128), CAAWDDSLSHWL (SEQ ID NO:129), FSNAWMSWVRQVPG (SEQ ID NO:130), STLGGSGGGSTYYADSVKGR (SEQ ID NO:131), and AKLGGRSRYGRWPRQFDY (SEQ ID NO:132). In some embodiments, the humanized antibody comprises a light chain variable domain comprising (i) CDR-L1 comprising sequence CSGSSSNIGNNYVS (SEQ ID NO:127); (ii) CDR-L2 comprising sequence SNNQRPS (SEQ ID NO:128); and (iii) CDR-L3 comprising sequence CAAWDDSLSHWL (SEQ ID NO:129). In some embodiments, the humanized antibody comprises a heavy chain variable domain comprising (i) CDR-H1 comprising sequence FSNAWMSWVRQVPG (SEQ ID NO:130); (ii) CDR-H2 comprising sequence STLGGSGGGSTYYADSVKGR (SEQ ID NO:131); and (iii) CDR-H3 comprising sequence AKLGGRSRYGRWPRQFDY (SEQ ID NO:132). In some embodiments, the humanized antibody comprises a light chain variable domain comprising (i) CDR-L1 comprising sequence CSGSSSNIGNNYVS (SEQ ID NO:127); (ii) CDR-L2 comprising sequence SNNQRPS (SEQ ID NO:128); and (iii) CDR-L3 comprising sequence CAAWDDSLSHWL (SEQ ID NO:129) and a heavy chain variable domain comprising (i) CDR-H1 comprising sequence FSNAWMSWVRQVPG (SEQ ID NO:130); (ii) CDR-H2 comprising sequence STLGGSGGGSTYYADSVKGR (SEQ ID NO:131); and (iii) CDR-H3 comprising sequence AKLGGRSRYGRWPRQFDY (SEQ ID NO:132.

In some embodiments, the humanized antibody comprises one or more (at least one, two, three, four, five, or six) CDRs selected from the group consisting of CSGSSSNIGSNYVS (SEQ ID NO:133), GNYNRPS (SEQ ID NO:134), CAAWDDSLSGWV (SEQ ID NO:135), FSSYWMSWVRQAPG (SEQ ID NO:136), SSISGSGRRTYYADSVQGR (SEQ ID NO:137), and ARLVSYGSGSFGFDY (SEQ ID NO:138). In some embodiments, the humanized antibody comprises a light chain variable domain comprising (i) CDR-L1 comprising sequence CSGSSSNIGSNYVS (SEQ ID NO:133); (ii) CDR-L2 comprising sequence GNYNRPS (SEQ ID NO:134); and (iii) CDR-L3 comprising sequence CAAWDDSLSGWV (SEQ ID NO:135). In some embodiments, the humanized antibody comprises a heavy chain variable domain comprising (i) CDR-H1 comprising sequence FSSYWMSWVRQAPG (SEQ ID NO:136); (ii) CDR-H2 comprising sequence SSISGSGRRTYYADSVQGR (SEQ ID NO:137); and (iii) CDR-H3 comprising sequence ARLVSYGSGSFGFDY (SEQ ID NO:138). In some embodiments, the humanized antibody comprises a light chain variable domain comprising (i) CDR-L1 comprising sequence CSGSSSNIGSNYVS (SEQ ID NO:133); (ii) CDR-L2 comprising sequence GNYNRPS (SEQ ID NO:134); and (iii) CDR-L3 comprising sequence CAAWDDSLSGWV (SEQ ID NO:135) and a heavy chain variable domain comprising (i) CDR-H1 comprising sequence FSSYWMSWVRQAPG (SEQ ID NO:136); (ii) CDR-H2 comprising sequence SSISGSGRRTYYADSVQGR (SEQ ID NO:137); and (iii) CDR-H3 comprising sequence ARLVSYGSGSFGFDY (SEQ ID NO:138).

In some embodiments, the humanized antibody comprises one or more (at least one, two, three, four, five, or six) CDRs selected from the group consisting of CSGRSSNIGNSYVS (SEQ ID NO:139), RNNQRPS (SEQ ID NO:140), CAGWDDTLRAWV (SEQ ID NO:141), FRDYYVSWIRQAPG (SEQ ID NO:142), SSISGSGGRTYYADSVEGR (SEQ ID NO:143), and ARVSALRRPMTTVTTYWFDP (SEQ ID NO:144). In some embodiments, the humanized antibody comprises a light chain variable domain comprising (i) CDR-L1 comprising sequence CSGRSSNIGNSYVS (SEQ ID NO:139); (ii) CDR-L2 comprising sequence RNNQRPS (SEQ ID NO:140); and (iii) CDR-L3 comprising sequence CAGWDDTLRAWV (SEQ ID NO:141). In some embodiments, the humanized antibody comprises a heavy chain variable domain comprising (i) CDR-H1 comprising sequence FRDYYVSWIRQAPG (SEQ ID NO:142); (ii) CDR-H2 comprising sequence SSISGSGGRTYYADSVEGR (SEQ ID NO:143); and (iii) CDR-H3 comprising sequence ARVSALRRPMTTVTTYWFDP (SEQ ID NO:144). In some embodiments, the humanized antibody comprises a light chain variable domain comprising (i) CDR-L1 comprising sequence CSGRSSNIGNSYVS (SEQ ID NO:139); (ii) CDR-L2 comprising sequence RNNQRPS (SEQ ID NO:140); and (iii) CDR-L3 comprising sequence CAGWDDTLRAWV (SEQ ID NO:141) and a heavy chain variable domain comprising (i) CDR-H1 comprising sequence FRDYYVSWIRQAPG (SEQ ID NO:142); (ii) CDR-H2 comprising sequence SSISGSGGRTYYADSVEGR (SEQ ID NO:143); and (iii) CDR-H3 comprising sequence ARVSALRRPMTTVTTYWFDP (SEQ ID NO:144).

### (v) Human Antibodies

In some embodiments, the anti-oxidized LDL antibodies are human antibodies. As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy chain joining region (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA 90:2551 (1993); Jakobovits et al., Nature 362:255-258 (1993); Bruggermann et al., Year in Immuno. 7:33 (1993); and U.S. Pat. Nos. 5,591,669; 5,589,369; and 5,545,807.

Alternatively, phage display technology (McCafferty et al., Nature 348:552-553 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats; for their review see, e.g., Johnson, Kevin S. and Chiswell, David J., Current Opinion in Structural Biology 3:564-571 (1993). Several sources of V-gene segments can be used for phage display. Clackson et al., Nature 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222:581-597 (1991), or Griffith et al., EMBO J. 12:725-734 (1993). See also, U.S. Pat. Nos. 5,565,332 and 5,573,905.

Human antibodies may also be generated by in vitro activated B cells (see U.S. Pat. Nos. 5,567,610 and 5,229,275).

In some embodiments, the human antibody comprises one or more (at least one, two, three, four, five, or six) CDRs from IEIA8, IEI-G8, IEI-D8, KTT-D6, 1-B12, 1-C07, 1-C12, 1-G10, 2-F07, 2-F09, 4-A02, IEI-E3, 2D03, LDO-D4, and/or KTT-B8. In some embodiments, the human antibody comprises one or more (at least one, two, three, four, five, or six) CDRs from IEI-E3, 2D03, LDO-D4, and/or KTT-B8.

In some embodiments, the human antibody comprises a light chain variable domain or heavy chain variable domain selected from Table 2. In some embodiments, the human antibody comprises a heavy chain variable domain selected from the group consisting of SEQ ID NO:64, SEQ ID NO:68, SEQ ID NO:72, SEQ ID NO:76, SEQ ID NO:80, SEQ ID NO:84, SEQ ID NO:88, SEQ ID NO:92, SEQ ID NO:96, SEQ ID NO:100, SEQ ID NO:104, SEQ ID NO:108, SEQ ID NO:112, SEQ ID NO:116, SEQ ID NO:120, and SEQ ID NO:124. In some embodiments, the human antibody comprises a light chain variable domain selected from the group consisting of SEQ ID NO:66, SEQ ID NO:70, SEQ ID NO:74, SEQ ID NO:78, SEQ ID NO:82, SEQ ID NO:86, SEQ ID NO:90, SEQ ID NO:94, SEQ ID NO:98, SEQ ID NO:102, SEQ ID NO:106, SEQ ID NO:110, SEQ ID NO:114, SEQ ID NO:118, SEQ ID NO:122, and SEQ ID NO:126. In some embodiments, the human antibody comprises a heavy chain variable domain comprising SEQ ID NO:104 and a light chain variable domain comprising SEQ ID NO:106. In some embodiments, the human antibody comprises a heavy chain variable domain comprising SEQ ID NO:68 and a light chain variable domain comprising SEQ ID NO:70. In some embodiments, the human antibody comprises a heavy chain variable domain comprising SEQ ID NO:96 and a light chain variable domain comprising SEQ ID NO:98.

In some embodiments, the human antibody comprises one or more (at least one, two, three, four, five, or six) CDRs from a sequence selected from the group consisting of SEQ ID NO:64, SEQ ID NO:68, SEQ ID NO:72, SEQ ID NO:76, SEQ ID NO:80, SEQ ID NO:84, SEQ ID NO:88, SEQ ID NO:92, SEQ ID NO:96, SEQ ID NO:100, SEQ ID NO:104, SEQ ID NO:108, SEQ ID NO:112, SEQ ID NO:116, SEQ ID NO:120, SEQ ID NO:124, SEQ ID NO:66, SEQ ID NO:70, SEQ ID NO:74, SEQ ID NO:78, SEQ ID NO:82, SEQ ID NO:86, SEQ ID NO:90, SEQ ID NO:94, SEQ ID NO:98, SEQ ID NO:102, SEQ ID NO:106, SEQ ID NO:110, SEQ ID NO:114, SEQ ID NO:118, SEQ ID NO:122, and SEQ ID NO:126. In some embodiments, the human antibody comprises one or more (at least one, two, three, four, five, or six) CDRs derived from one or more V_{H} and/or V_{L} sequences of the antibodies in FIG. 3 of WO 2004/030607, which is incorporated by reference in its entirety. In some embodiments, the human antibody comprises one or more (at least one, two, three, four, five, or six) CDRs of the antibodies of Table 2 of WO 2007/025781, which is incorporated by reference in its entirety.

TABLE 2
IEIA8
Variable heavy (VH) region
(SEQ ID NO: 63)
GAGGTGCAGCTGTTGGAGTCTGGGGGAGGCTTGGTACAGCCTGGGGGGTC
CCTGAGACTCTCCTGTGCAGCCTCTGGATTCACCTTCAATAACGCCTGGA
TGAGCTGGGTCCGCCAGGCTCCAGGGAAGGGGCTGGAGTGGGTCTCATCC
ATTAGTAGTAGTAGTAGTTACATATACTACGCAGACTCAGTGAAGGGCCG
ATTCACCATCTCCAGAGACAATTCCAAGAACACGCTGTATCTGCAAATGA
ACAGCCTGAGAGCCGAGGACACTGCCGTGTATTACTGTGCGAGAGTCAGT
AGGTACTACTACGGACCATCTTTCTACTTTGACTCCTGGGGCCAGGGTAC
ACTGGTCACCGTGAGCAGC
Variable heavy (VH) region
(SEQ ID NO: 64)
EVQLLESGGGLVQPGGSLRLSCAASGFTFNNAWMSWVRQAPGKGLEWVSS
ISSSSSYIYYADSVKGRFTISRDNSKNTLYLQMNSLRAEDTAVYYCARVS
RYYYGPSFYFDSWGQGTLVTVSS
Variable light (VL) region
(SEQ ID NO: 65)
CAGTCTGTGCTGACTCAGCCACCCTCAGCGTCTGGGACCCCCGGGCAGAG
GGTCACCATCTCCTGCTCTGGAAGCAGGTCCAACATTGGGAATAATTATG
TATCCTGGTATCAGCAGCTCCCAGGAACGGCCCCCAAACTCCTCATCTAT
GGTAACAACAATCGGCCCTCAGGGGTCCCTGACCGATTCTCTGGCTCCAA
GTCTGGCACCTCAGCCTCCCTGGCCATCAGTGGGCTCCGGTCCGAGGATG
AGGCTGATTATTACTGTGCAGCATGGGATGACAGCCTGAATGGTCATTGG
GTGTTCGGCGGAGGAACCAAGCTGACGGTCCTAGGT
Variable light (VL) region
(SEQ ID NO: 66)
QSVLTQPPSASGTPGQRVTISCSGSRSNIGNNYVSWYQQLPGTAPKLLIY
GNNNRPSGVPDRFSGSKSGTSASLAISGLRSEDEADYYCAAWDDSLNGHW
VFGGGTKLTVLG
IEI-E3
Variable heavy (VH) region
(SEQ ID NO: 67)
GAGGTGCAGCTGTTGGAGTCTGGGGGAGGCTTGGTACAGCCTGGGGGGTC
CCTGAGACTCTCCTGTGCGGCCTCTGGATTCACCTTCAGTGACTACTACA
TGAGCTGGGTCCGCCAGGCTCCCGGGAAGGGGCTGGAGTGGGTATCGGGT
GTTAGTTGGAATGGCAGTAGGACGCACTATGCAGACTCTGTGAAGGGCCG
ATTCACCATCTCCAGAGACAATTCCAAGAACACGCTGTATCTGCAAATGA
ACAGCCTGAGAGCCGAGGACACTGCCGTGTATTACTGTGCGAGAGCGGCT
AGGTACTCCTACTACTACTACGGTATGGACGTCTGGGGCCAAGGTACACT
GGTCACCGTGAGCAGC
Variable heavy (VH) region
(SEQ ID NO: 68)
EVQLLESGGGLVQPGGSLRLSCAASGFTFSDYYMSWVRQAPGKGLEWVSG
VSWNGSRTHYADSVKGRFTISRDNSKNTLYLQMNSLRAEDTAVYYCARAA
RYSYYYYGMDVWGQGTLVTVSS
Variable light (VL) region
(SEQ ID NO: 69)
CAGTCTGTGCTGACTCAGCCACCCTCAGCGTCTGGGACCCCCGGGCAGAG
GGTCACCATCTCCTGTTCTGGAAGCAGCTCCAACATCGGAAATAATGCTG
TAAACTGGTATCAGCAGCTCCCAGGAACGGCCCCCAAACTCCTCATCTAT
GGGAATGATCGGCGGCCCTCAGGGGTCCCTGACCGATTCTCTGGCTCCAA
GTCTGGCACCTCAGCCTCCCTGGCCATCAGTGGGCTCCGGTCCGAGGATG
AGGCTGATTATTACTGTCAGACCTGGGGCACTGGCCGGGGGGTATTCGGC
GGAGGAACCAAGCTGACGGTCCTAGGT
Variable light (VL) region
(SEQ ID NO: 70)
QSVLTQPPSASGTPGQRVTISCSGSSSNIGNNAVNWYQQLPGTAPKLLIY
GNDRRPSGVPDRFSGSKSGTSASLAISGLRSEDEADYYCQTWGTGRGVFG
GGTKLTVLG
IEI-G8
Variable heavy (VH) region
(SEQ ID NO: 71)
GAGGTGCAGCTGTTGGAGTCTGGGGGAGGCTTGGTACAGCCTGGGGGGTC
CCTGAGACTCTCCTGTGCAGCCTCTGGATTCACCTTTAGTAGCTATTGGA
TGAGCTGGGTCCGCCAGGCTCCAGGGAAGGGGCTGGAGTGGGTCTCAAGT
ATCAGTGGTAGTGGTCGTAGGACATACTACGCAGACTCCGTGCAGGGCCG
GTTCACCATCTCCAGAGACAATTCCAAGAACACGCTGTATCTGCAAATGA
ACAGCCTGAGAGCCGAGGACACTGCCGTGTATTACTGTGCGAGATTGGTC
TCCTATGGTTCGGGGAGTTTCGGTTTTGACTACTGGGGCCAAGGTACACT
GGTCACCGTGAGCAGC
Variable heavy (VH) region
(SEQ ID NO: 72)
EVQLLESGGGLVQPGGSLRLSCAASGFTFSSYWMSWVRQAPGKGLEWVSS
ISGSGRRTYYADSVQGRFTISRDNSKNTLYLQMNSLRAEDTAVYYCARLV
SYGSGSFGFDYWGQGTLVTVSS
Variable light (VL) region
(SEQ ID NO: 73)
CAGTCTGTGCTGACTCAGCCACCCTCAGCGTCTGGGACCCCCGGGCAGAG
GGTCACCATCTCTTGTTCTGGAAGCAGCTCCAATATCGGAAGTAATTATG
TATCCTGGTATCAGCAGCTCCCAGGAACGGCCCCCAAACTCCTCATCTAT
GGTAACTACAATCGGCCCTCAGGGGTCCCTGACCGATTCTCTGGCTCCAA
GTCTGGCACCTCAGCCTCCCTGGCCATCAGTGGGCTCCGGTCCGAGGATG
AGGCTGATTATTACTGTGCAGCATGGGATGACAGCCTGAGTGGTTGGGTG
TTCGGCGGAGGAACCAAGCTGACGGTCCTAGGT
Variable light (VL) region
(SEQ ID NO: 74)
QSVLTQPPSASGTPGQRVTISCSGSSSNIGSNYVSWYQQLPGTAPKLLIY
GNYNRPSGVPDRFSGSKSGTSASLAISGLRSEDEADYYCAAWDDSLSGWV
FGGGTKLTVL
IEI-D8
Variable heavy (VH) region
(SEQ ID NO: 75)
GAGGTGCAGCTGTTGGAGTCTGGGGGAGGCTTGGTACAGCCTGGGGGGTC
CCTGAGACTCTCCTGTGCAGCCTCTGGATTCACCTTCAGTAACGCCTGGA
TGAGCTGGGTCCGCCAGGTTCCAGGGAAGGGGCTGGAGTGGGTCTCAACT
CTTGGTGGTAGTGGTGGTGGTAGCACATACTACGCAGACTCCGTGAAGGG
CCGGTTCACCATCTCCAGAGACAATTCCAAGAACACGCTGTATCTGCAAA
TGAACAGCCTGAGAGCCGAGGACACTGCCGTGTATTACTGTGCGAAGTTA
GGGGGGCGATCCCGATATGGGCGGTGGCCCCGCCAATTTGACTACTGGGG
CCAAGGTACACTGGTCACCGTGAGCAGC
Variable heavy (VH) region
(SEQ ID NO: 76)
EVQLLESGGGLVQPGGSLRLSCAASGFTFSNAWMSWVRQVPGKGLEWVST
LGGSGGGSTYYADSVKGRFTISRDNSKNTLYLQMNSLRAEDTAVYYCAKL
GGRSRYGRWPRQFDYWGQGTLVTVSS
Variable light (VL) region
(SEQ ID NO: 77)
CAGTCTGTGCTGACTCAGCCACCCTCAGCGTCTGGGACCCCCGGGCAGAG
GGTCACCATCTCCTGCTCTGGAAGCAGCTCCAACATTGGAAATAACTATG
TATCCTGGTATCAGCAGCTCCCAGGAACGGCCCCCAAACTCCTCATCTAT
AGTAATAATCAGCGGCCCTCAGGGGTCCCTGACCGATTCTCTGGCTCCAA
GTCTGGCACCTCAGCCTCCCTGGCCATCAGTGGGCTCCGGTCCGAGGATG
AGGCTGATTATTACTGTGCAGCATGGGATGACAGCCTGAGTCATTGGCTG
TTCGGCGGAGGAACCAAGCTGACGGTCCTAGGT
Variable light (VL) region
(SEQ ID NO: 78)
QSVLTQPPSASGTPGQRVTISCSGSSSNIGNNYVSWYQQLPGTAPKLLIY
SNNQRPSGVPDRFSGSKSGTSASLAISGLRSEDEADYYCAAWDDSLSHWL
FGGGTKLTVL
KTT-D6
Variable heavy (VH) region
(SEQ ID NO: 79)
GAGGTGCAGCTGTTGGAGTCTGGGGGAGGCTTGGTACAGCCTGGGGGGTC
CCTGAGACTCTCCTGTGCAGCCTCTGGATTCACCTTCAGTGACTACTACA
TGAGCTGGATCCGCCAGGCTCCAGGGAAGGGGCTGGAGTGGGTCTCAAGT
ATCAGTGGCCGTGGGGGTAGTTCCTACTACGCAGACTCCGTGAGGGGCCG
GTTCACCATCTCCAGAGACAATTCCAAGAACACGCTGTATCTGCAAATGA
ACAGCCTGAGAGCCGAGGACACTGCCGTGTATTACTGTGCGAGACTTTCC
TACAGCTATGGTTACGAGGGGGCCTACTACTTTGACTACTGGGGCCAGGG
TACACTGGTCACCGTGAGCAGC
Variable heavy (VH) region
(SEQ ID NO: 80)
EVQLLESGGGLVQPGGSLRLSCAASGFTFSDYYMSWIRQAPGKGLEWVSS
ISGRGGSSYYADSVRGRFTISRDNSKNTLYLQMNSLRAEDTAVYYCARLS
YSYGYEGAYYFDYWGQGTLVTVSS
Variable light (VL) region
(SEQ ID NO: 81)
CAGTCTGTGCTGACTCAGCCACCCTCAGCGTCTGGGACCCCCGGGCAGAG
GGTCACCATCTCCTGCTCTGGAAGCAGCTCCAACATTGGGAATAATTATG
TATCCTGGTATCAGCAGCTCCCAGGAACGGCCCCCAAACTCCTCATCTAT
AGGAATAATCAGCGGCCCTCAGGGGTCCCTGACCGATTCTCTGGCTCCAA
GTCTGGCACCTTAGCCTCCCTGGCCATCAGTGGGCTCCGGTCCGAGGATG
AGGCTGATTATTACTGTGCAACCTGGGATGACAGCCTGAATGGTTGGGTG
TTCGGCGGAGGAACCAAGCTGACGGTCCTAGGT
Variable light (VL) region
(SEQ ID NO: 82)
QSVLTQPPSASGTPGQRVTISCSGSSSNIGNNYVSWYQQLPGTAPKLLIY
RNNQRPSGVPDRFSGSKSGTLASLAISGLRSEDEADYYCATWDDSLNGWV
FGGGTKLTVLG
KTT-B8
Variable heavy (VH) region
(SEQ ID NO: 83)
GAGGTGCAGCTGTTGGAGTCTGGGGGAGGCTTGGTACAGCCTGGGGGGTC
CCTGAGACTCTCCTGTGCAGCCTCTGGATTCACCTTTAGCAGCTATGCCA
TGAGCTGGGTCCGCCAGGCTCCAGGGAAGGGGCTGGAGTGGGTCTCATCC
ATTAGTAGTAGTGGTCGTTTCATTTACTACGCAGACTCAATGAAGGGCCG
CTTCACCATCTCCAGAGACAATTCCAAGAACACGCTGTATCTGCAAATGA
ACAGCCTGAGAGCCGAGGACACTGCCGTGTATTACTGTACGAGGCTCCGG
AGAGGGAGCTACTTCTGGGCTTTTGATATCTGGGGCCAAGGTACACTGGT
CACCGTGAGCAGC
Variable heavy (VH) region
(SEQ ID NO: 84)
EVQLLESGGGLVQPGGSLRLSCAASGFTFSSYAMSWVRQAPGKGLEWVSS
ISSSGRFIYYADSMKGRFTISRDNSKNTLYLQMNSLRAEDTAVYYCTRLR
RGSYFWAFDIWGQGTLVTVSS
Variable light (VL) region
(SEQ ID NO: 85)
CAGTCTGTGCTGACTCAGCCACCCTCAGCGTCTGGGACCCCCGGGCAGAG
GGTCACCATCTCCTGTTCTGGAAGCAGCTCCAACATTGGCGGTGAGTCTG
TATCCTGGTATCAGCAGCTCCCAGGAACGGCCCCCAAACTCCTCATCTAT
AGTAATAATCAGCGGCCCTCAGGGGTCCCTGACCGATTCTCTGGCTCCAA
GTCTGGCACCTCAGCCTCCCTGGCCATCAGTGGGCTCCGGTCCGAGGATG
AGGCTGATTATTACTGTGCAGCATGGGATGACAGCCTGAATGGTTGGGTG
TTCGGCGGAGGAACCAAGCTGACGGTCCTAGGT
Variable light (VL) region
(SEQ ID NO: 86)
QSVLTQPPSASGTPGQRVTISCSGSSSNIGGESVSWYQQLPGTAPKLLIY
SNNQRPSGVPDRFSGSKSGTSASLAISGLRSEDEADYYCAAWDDSLNGWV
FGGGTKLTVLG
1-B12
Variable heavy (VH) region
(SEQ ID NO: 87)
GAGGTGCAGCTGTTGGAGTCTGGGGGAGGCTTGGTACAGCCTGGGGGGTC
CCTGAGACTCTCCTGTGCAGCCTCTGGATTCACCTTTAGAACGTATTGGA
TGACCTGGGTCCGCCAGGCTCCAGGGAAGGGGCTGGAGTGGGTCTCATCT
ATTAGCAGTAGCAGTAATTACATATTCTACGCAGACTCAGTGAAGGGCCG
ATTCACCATCTCCAGAGACAATTCCAAGAACACGCTGTATCTGCAAATGA
ACAGCCTGAGAGCCGAGGACACTGCCGTGTATTACTGTGCGAGACTCAGA
CGGAGCAGCTGGTACGGGGGGTACTGGTTCGACCCCTGGGGCCAAGGTAC
ACTGGTCACCGTGAGCTCA
Variable heavy (VH) region
(SEQ ID NO: 88)
EVQLLESGGGLVQPGGSLRLSCAASGFTFRTYWMTWVRQAPGKGLEWVSS
ISSSSNYIFYADSVKGRFTISRDNSKNTLYLQMNSLRAEDTAVYYCARLR
RSSWYGGYWFDPWGQGTLVTVSS
Variable light (VL) region
(SEQ ID NO: 89)
CAGTCTGTGCTGACTCAGCCACCCTCAGCGTCTGGGACCCCCGGGCAGAG
GGTCACCATCTCCTGCTCTGGAAGCAGCTCCAACATTGGGAATAATTATG
TATCCTGGTATCAGCAGCTCCCAGGAACGGCCCCCAAACTCCTCATCTAT
AGGAATAATCAGCGGCCCTCAGGGGTCCCTGACCGATTCTCTGGCTCCAA
GTCTGGCACCTCAGCCTCCCTGGCCATCAGTGGGCTCCGGTCCGAGGATG
AGGCTGATTATTACTGTGCAGCATGGGATGACAGCCTGAATGGTCATTGG
GTGTTCGGCGGAGGAACCAAGCTGACGGTCCTAGGT
Variable light (VL) region
(SEQ ID NO: 90)
QSVLTQPPSASGTPGQRVTISCSGSSSNIGNNYVSWYQQLPGTAPKLLIY
RNNQRPSGVPDRFSGSKSGTSASLAISGLRSEDEADYYCAAWDDSLNGHW
VFGGGTKLTVLG
1-C07
Variable heavy (VH) region
(SEQ ID NO: 91)
GAGGTGCAGCTGTTGGAGTCTGGGGGAGGCTTGGTACAGCCTGGGGGGTC
CCTGAGACTCTCCTGTGCAGCCTCTGGATTCACCTTCAGTAGCAACTACA
TGAGCTGGGTCCGCCAGGCTCCAGGGAAGGGGCTGGAGTGGGTCTCATCC
ATTAGTAGTAGTAGTAGTTACATATACTACGCAGACTCAGTGAAGGGCCG
ATTCACCATCTCCAGAGACAATTCCAAGAACACGCTGTATCTGCAAATGA
ACAGCCTGAGAGCCGAGGACACTGCCGTGTATTACTGTGCGAGAGTAGGC
CGGTATAACTGGAAGACGGGGCATGCTTTTGATATCTGGGGCCAGGGTAC
ACTGGTCACCGTGAGCTCA
Variable heavy (VH) region
(SEQ ID NO: 92)
EVQLLESGGGLVQPGGSLRLSCAASGFTFSSNYMSWVRQAPGKGLEWVSS
ISSSSSYIYYADSVKGRFTISRDNSKNTLYLQMNSLRAEDTAVYYCARVG
RYNWKTGHAFDIWGQGTLVTVSS
Variable light (VL) region
(SEQ ID NO: 93)
CAGTCTGTGCTGACTCAGCCACCCTCAGCGTCTGGGACCCCCGGGCAGAG
GGTCACCATCTCCTGCTCTGGAAGGACCTACAACATTGGAAATAATTATG
TATCGTGGTATCAGCAGCTCCCAGGAACGGCCCCCAAACTCCTCATCTAT
GGTAACATCAATCGGCCCTCAGGGGTCCCTGACCGATTCTCTGGCTCCAA
GTCTGGCACCTCAGCCTCCCTGGCCATCAGTGGGCTCCGGTCCGAGGATG
AGGCTGATTATTACTGTGCAGCATGGGATGTCAGGCTGAATGGTTGGGTG
TTCGGCGGAGGAACCAAGCTGACGGTCCTAGGT
Variable light (VL) region
(SEQ ID NO: 94)
QSVLTQPPSASGTPGQRVTISCSGRTYNIGNNYVSWYQQLPGTAPKLLIY
GNINRPSGVPDRFSGSKSGTSASLAISGLRSEDEADYYCAAWDVRLNGWV
FGGGTKLTVLG
1-C12
Variable heavy (VH) region
(SEQ ID NO: 95)
GAGGTGCAGCTGTTGGAGTCTGGGGGAGGCTTGGTACAGCCTGGGGGGTC
CCTGAGACTCTCCTGTGCAGCCTCTGGATTCACCTTCCGTGACTACTACG
TGAGCTGGATCCGCCAGGCTCCAGGGAAGGGGCTGGAGTGGGTCTCAAGT
ATTAGTGGTAGTGGGGGTAGGACATACTACGCAGACTCCGTGGAGGGCCG
GTTCACCATCTCCAGAGACAATTCCAAGAACACGCTGTATCTGCAAATGA
ACAGCCTGAGAGCCGAGGACACTGCCATGTATTACTGTGCCAGAGTATCC
GCCCTTCGGAGACCCATGACTACAGTAACTACTTACTGGTTCGACCCCTG
GGGCCAAGGTACACTGGTCACCGTGAGCTCA
Variable heavy (VH) region
(SEQ ID NO: 96)
EVQLLESGGGLVQPGGSLRLSCAASGFTFRDYYVSWIRQAPGKGLEWVSS
ISGSGGRTYYADSVEGRFTISRDNSKNTLYLQMNSLRAEDTAMYYCARVS
ALRRPMTTVTTYWFDPWGQGTLVTVSS
Variable light (VL) region
(SEQ ID NO: 97)
CAGTCTGTGCTGACTCAGCCACCCTCAGCGTCTGGGACCCCCGGGCAGAG
GGTCACCATCTCCTGCTCTGGAAGGAGCTCCAACATTGGGAATAGTTATG
TCTCCTGGTATCAGCAGCTCCCAGGAACGGCCCCCAAACTCCTCATCTAT
AGGAATAATCAGCGGCCCTCAGGGGTCCCTGACCGATTCTCTGGCTCCAA
GTCTGGCACCTCAGCCTCCCTGGCCATCAGTGGGCTCCGGTCCGAGGATG
AGGCTGATTATTACTGTGCAGGATGGGATGACACCCTGCGTGCTTGGGTG
TTCGGCGGAGGAACCAAGCTGACGGTCCTAGGT
Variable light (VL) region
(SEQ ID NO: 98)
QSVLTQPPSASGTPGQRVTISCSGRSSNIGNSYVSWYQQLPGTAPKLLIY
RNNQRPSGVPDRFSGSKSGTSASLAISGLRSEDEADYYCAGWDDTLRAWV
FGGGTKLTVLG
1-G10
Variable heavy (VH) region
(SEQ ID NO: 99)
GAGGTGCAGCTGTTGGAGTCTGGGGGAGGCTTGGTACAGCCTGGGGGGTC
CCTGAGACTCTCCTGTGCAGCCTCTGGATTCACCTTCAGTAACGCCTGGA
TGAGCTGGGTCCGCCAGGCTCCAGGGAAGGGGCTGGAGTGGGTCTCCGCT
ATTAGTGGTAGTGGTAACACATACTATGCAGACTCCGTGAAGGGCCGGTT
CACCATCTCCAGAGACAATTCCAAGAACACGCTGTATCTGCAAATGAACA
GCCTGAGAGCCGAGGACACTGCCGTGTATTACTGTGCGAGAGCCTCCCAC
CGTATATTAGGTTATGCTTTTGATATCTGGGGCCAGGGTACACTGGTCAC
CGTGAGCTCA
Variable heavy (VH) region
(SEQ ID NO: 100)
EVQLLESGGGLVQPGGSLRLSCAASGFTFSNAWMSWVRQAPGKGLEWVSA
ISGSGNTYYADSVKGRFTISRDNSKNTLYLQMNSLRAEDTAVYYCARASH
RILGYAFDIWGQGTLVTVSS
Variable light (VL) region
(SEQ ID NO: 101)
CAGTCTGTGCTGACTCAGCCACCCTCAGCGTCTGGGACCCCCGGGCAGAG
GGTCACCATCTCTTGTTCTGGAAGCCGCTCCAACATCGGGAGAAATGCTG
TTAGTTGGTATCAGCAGCTCCCAGGAACGGCCCCCAAACTCCTCATCTAT
GCTAACAGCAATCGGCCCTCAGGGGTCCCTGACCGATTCTCTGGCTCCAA
GTCTGGCACCTCAGCCTCCCTGGCCATCAGTGGGCTCCGGTCCGAGGATG
AGGCTGATTATTACTGTGCAGCATGGGATGGCAGCCTGAATGGTTGGGTG
TTCGGCGGAGGAACCAAGCTGACGGTCC
Variable light (VL) region
(SEQ ID NO: 102)
QSVLTQPPSASGTPGQRVTISCSGSRSNIGRNAVSWYQQLPGTAPKLLIY
ANSNRPSGVPDRFSGSKSGTSASLAISGLRSEDEADYYCAAWDGSLNGWV
FGGGTKLTV
2-D03
Variable heavy (VH) region
(SEQ ID NO: 103)
GAGGTGCAGCTGTTGGAGTCTGGGGGAGGCTTGGTACAGCCTGGGGGGTC
CCTGAGACTCTCCTGTGCAGCCTCTGGATTCACCTTCAGTAACGCCTGGA
TGAGCTGGGTCCGCCAGGCTCCAGGGAAGGGGCTGGAGTGGGTCTCAAGT
ATTAGTGTTGGTGGACATAGGACATATTATGCAGATTCCGTGAAGGGCCG
GTCCACCATCTCCAGAGACAATTCCAAGAACACGCTGTATCTGCAAATGA
ACAGCCTGAGAGCCGAGGACACTGCCGTGTATTACTGTGCACGGATACGG
GTGGGTCCGTCCGGCGGGGCCTTTGACTACTGGGGCCAGGGTACACTGGT
CACCGTGAGCTCA
Variable heavy (VH) region
(SEQ ID NO: 104)
EVQLLESGGGLVQPGGSLRLSCAASGFTFSNAWMSWVRQAPGKGLEWVSS
ISVGGHRTYYADSVKGRSTISRDNSKNTLYLQMNSLRAEDTAVYYCARIR
VGPSGGAFDYWGQGTLVTVSS
Variable light (VL) region
(SEQ ID NO: 105)
CAGTCTGTGCTGACTCAGCCACCCTCAGCGTCTGGGACCCCCGGGCAGAG
GGTCACCATCTCCTGCTCTGGAAGCAACACCAACATTGGGAAGAACTATG
TATCTTGGTATCAGCAGCTCCCAGGAACGGCCCCCAAACTCCTCATCTAT
GCTAATAGCAATCGGCCCTCAGGGGTCCCTGACCGATTCTCTGGCTCCAA
GTCTGGCACCTCAGCCTCCCTGGCCATCAGTGGGCTCCGGTCCGAGGATG
AGGCTGATTATTACTGTGCGTCATGGGATGCCAGCCTGAATGGTTGGGTA
TTCGGCGGAGGAACCAAGCTGACGGTCCTAGGT
Variable light (VL) region
(SEQ ID NO: 106)
QSVLTQPPSASGTPGQRVTISCSGSNTNIGKNYVSWYQQLPGTAPKLLIY
ANSNRPSGVPDRFSGSKSGTSASLAISGLRSEDEADYYCASWDASLNGWV
FGGGTKLTVLG
2-F07
Variable heavy (VH) region
(SEQ ID NO: 107)
GAGGTGCAGCTGTTGGAGTCTGGGGGAGGCTTGGTACAGCCTGGGGGGTC
CCTGAGACTCTCCTGTGCAGCCTCTGGATTCACCTTCAGTAACGCCTGGA
TGAGCTGGGTCCGCCAGGCTCCAGGGAAGGGGCTGGAGTGGGTCTCATCC
ATTAGTAGTAGTAGTAGTTACATATACTACGCAGACTCAGTGAAGGGCCG
ATCCACCATCTCCAGAGACAATTCCAAGAACACGCTGTATCTGCAAATGA
ACAGCCTGAGAGCCGAGGACACTGCCGTGTATTACTGTGCGAGGCTCACA
AATATTTTGACTGGTTATTATACCTCAGGATATGCTTTTGATATCTGGGG
CCAAGGTACACTGGTCACCGTGAGCTCA
Variable heavy (VH) region
(SEQ ID NO: 108)
EVQLLESGGGLVQPGGSLRLSCAASGFTFSNAWMSWVRQAPGKGLEWVSS
ISSSSSYIYYADSVKGRSTISRDNSKNTLYLQMNSLRAEDTAVYYCARLT
NILTGYYTSGYAFDIWGQGTLVTVSS
Variable light (VL) region
(SEQ ID NO: 109)
CAGTCTGTGCTGACTCAGCCACCCTCAGCGTCTGGGACCCCCGGGCAGAG
GGTCACCATCTCCTGCTCTGGAAGCACCTCCAACATTGGGAAGAATTATG
TATCCTGGTATCAGCAGCTCCCAGGAACGGCCCCCAAACTCCTCATCTAT
GGTAACAGCAATCGGCCCTCAGGGGTCCCTGACCGATTCTCTGGCTCCAA
GTCTGGCACCTCAGCCTCCCTGGCCATCAGTGGGCTCCGGTCCGAGGATG
AGGCTGATTATTACTGTGCAGCATGGGATGCCAGCCTCAGTGGTTGGGTG
TTCGGCGGAGGAACCAAGCTGACGGTCCTAGGT
Variable light (VL) region
(SEQ ID NO: 110)
QSVLTQPPSASGTPGQRVTISCSGSTSNIGKNYVSWYQQLPGTAPKLLIY
GNSNRPSGVPDRFSGSKSGTSASLAISGLRSEDEADYYCAAWDASLSGWV
FGGGTKLTVLG
2-F09
Variable heavy (VH) region
(SEQ ID NO: 111)
GAGGTGCAGCTGTTGGAGTCTGGGGGAGGCTTGGTACAGCCTGGGGGGTC
CCTGAGACTCTCCTGTGCAGCCTCTGGATTCACCTTCAGTAGTTCTTGGA
TGAGTTGGGTCCGCCAGGCTCCAGGGAAGGGGCTGGAGTGGGTCTCATCC
ATTAGTAGTAGTAGTAGTTACATATACTACGCAGACTCAGTGAAGGGCCG
ATTCACCATCTCCAGAGACAATTCCAAGAACACGCTGTATCTGCAAATGA
ACAGCCTGAGAGCCGAGGACACTGCCGTGTATTACTGTGCGAGAGTAGGG
AACTACGGTTTCTACCACTACATGGACGTCTGGGGCCAAGGTACACTGGT
CACCGTGAGCTCA
Variable heavy (VH) region
(SEQ ID NO: 112)
EVQLLESGGGLVQPGGSLRLSCAASGFTFSSSWMSWVRQAPGKGLEWVSS
ISSSSSYIYYADSVKGRFTISRDNSKNTLYLQMNSLRAEDTAVYYCARVG
NYGFYHYMDVWGQGTLVTVSS
Variable light (VL) region
(SEQ ID NO: 113)
CAGTCTGTGCTGACTCAGCCACCCTCAGCGTCTGGGACCCCCGGGCAGAG
GGTCACCATCTCTTGTTCTGGAGGCAGCTCAAACATCGGAAAAAGAGGTG
TAAATTGGTATCAGCAGCTCCCAGGAACGGCCCCCAAACTCCTCATCTAT
GGTAACAGAAATCGGCCCTCAGGGGTCCCTGACCGATTCTCTGGCTCCAA
GTCTGGCACCTCAGCCTCCCTGGCCATCAGTGGGCTCCGGTCCGAGGATG
AGGCTGATTATTACTGTGCTACATGGGATTACAGCCTCAATGCTTGGGTG
TTCGGCGGAGGAACCAAGCTGACGGTCCTAGGT
Variable light (VL) region
(SEQ ID NO: 114)
QSVLTQPPSASGTPGQRVTISCSGGSSNIGKRGVNWYQQLPGTAPKLLIY
GNRNRPSGVPDRFSGSKSGTSASLAISGLRSEDEADYYCATWDYSLNAWV
FGGGTKLTVLG
4-A02
Variable heavy (VH) region
(SEQ ID NO: 115)
GAGGTGCAGCTGTTGGAGTCTGGGGGAGGCTTGGTACAGCCTGGGGGGTC
CCTGAGACTCTCCTGTGCAGCCTCTGGATTCACCTTTAGTAGCTATTGGA
TGAGCTGGGTCCGCCAGGCTCCAGGGAAGGGGCTGGAGTGGGTCTCATCC
ATTAGTAGTAGTAGTAGTTACATATACTACGCAGACTCAGTGAAGGGCCG
ATTCACCATCTCCAGAGACAATTCCAAGAACACGCTGTATCTGCAAATGA
ACAGCCTGAGAGCCGAGGACACTGCCGTGTATTACTGTGCGAGAATTAAA
CGGTTACGATTCGGCTGGACCCCTTTTGACTACTGGGGCCAGGGTACACT
GGTCACCGTGAGCTCA
Variable heavy (VH) region
(SEQ ID NO: 116)
EVQLLESGGGLVQPGGSLRLSCAASGFTFSSYWMSWVRQAPGKGLEWVSS
ISSSSSYIYYADSVKGRFTISRDNSKNTLYLQMNSLRAEDTAVYYCARIK
RLRFGWTPFDYWGQGTLVTVSS
Variable light (VL) region
(SEQ ID NO: 117)
CAGTCTGTTCTGACTCAGCCACCCTCAGCGTCTGGGACCCCCGGGCAGAG
GGTCACCATCTCCTGTTCTGGAAGCAGCTCCAACATCGGAAATAATGGTG
TAAACTGGTATCAGCAGCTCCCAGGAACGGCCCCCAAACTCCTCATCTAT
GGTAACAACAATCGGCCCTCAGGGGTCCCTGACCGATTCTCTGGCTCCAA
GTCTGGCACCTCAGCCTCCCTGGCCATCAGTGGGCTCCGGTCCGAGGATG
AGGCTGATTATTACTGTGCAGCATGGGATGACAGCCTGCGTGGTTGGCTG
TTCGGCGGAGGAACCAAGCTGACGGTCCTAGGT
Variable light (VL) region
(SEQ ID NO: 118)
QSVLTQPPSASGTPGQRVTISCSGSSSNIGNNGVNWYQQLPGTAPKLLIY
GNNNRPSGVPDRFSGSKSGTSASLAISGLRSEDEADYYCAAWDDSLRGWL
FGGGTKLTVLG
4-C03
Variable heavy (VH) region
(SEQ ID NO: 119)
GAGGTGCAGCTGTTGGAGTCTGGGGGAGGCTTGGTACAGCCTGGGGGGTC
CCTGAGACTCTCCTGTGCAGCCTCTGGATTCACCTTCAGTAACGCCTGGA
TGAGCTGGGTCCGCCAGGCTCCAGGGAAGGGGCTGGAGTGGGTCTCATCC
ATTAGTAGTAGTAGTAGTTACATATACTACGCAGACTCAGTGAAGGGCCG
ATTCACCATCTCCAGAGACAATTCCAAGAACACGCTGTATCTGCAAATGA
ACAGCCTGAGAGCCGAGGACACTGCCGTGTATTACTGTGCGAGAGTCAAT
AGCAAAAAGTGGTATGAGGGCTACTTCTTTGACTACTGGGGCCAGGGTAC
ACTGGTCACCGTGAGCTCA
Variable heavy (VH) region
(SEQ ID NO: 120)
EVQLLESGGGLVQPGGSLRLSCAASGFTFSNAWMSWVRQAPGKGLEWVSS
ISSSSSYIYYADSVKGRFTISRDNSKNTLYLQMNSLRAEDTAVYYCARVN
SKKWYEGYFFDYWGQGTLVTVSS
Variable light (VL) region
(SEQ ID NO: 121)
CAGTCTGTGCTGACTCAGCCACCCTCAGCGTCTGGGACCCCCGGGCAGAG
GGTCACCATCTCCTGCTCTGGAAGCAGCTCCAACATTGGGAATAATTATG
TATCCTGGTATCAGCAGCTCCCAGGAACGGCCCCCAAACTCCTCATCTAT
GGTAACAGCAATCGGCCCTCAGGGGTCCCTGACCGATTCTCTGGCTCCAA
GTCTGGCACCTCAGCCTCCCTGGCCATCAGTGGGCTCCGGTCCGAGGATG
AGGCTGATTATTACTGTGCAGCATGGGATGACAGTCTGAGTGGTTGGGTG
TTCGGCGGAGGAACCAAGCTGACGGTCCTAGGT
Variable light (VL) region
(SEQ ID NO: 122)
QSVLTQPPSASGTPGQRVTISCSGSSSNIGNNYVSWYQQLPGTAPKLLIY
GNSNRPSGVPDRFSGSKSGTSASLAISGLRSEDEADYYCAAWDDSLSGWV
FGGGTKLTVLG
4-D04
Variable heavy (VH) region
(SEQ ID NO: 123)
GAGGTGCAGCTGTTGGAGTCTGGGGGAGGCTTGGTACAGCCTGGGGGGTC
CCTGAGACTCTCCTGTGCAGCCTCTGGATTCACCTTCAGTAACGCCTGGA
TGAGCTGGGTCCGCCAGGCTCCAGGGAAGGGGCTGGAGTGGGTCTCATCC
ATTAGTACTAGTAGTAATTACATATACTACGCAGACTCAGTGAAGGGCCG
GTTCACCATCTCCAGAGACAATTCCAAGAACACGCTGTATCTGCAAATGA
ACAGCCTGAGAGCCGAGGACACTGCCGTGTATTACTGTGCGAGAGTCAAG
AAGTATAGCAGTGGCTGGTACTCGAATTATGCTTTTGATATCTGGGGCCA
AGGTACACTGGTCACCGTGAGCTCA
Variable heavy (VH) region
(SEQ ID NO: 124)
EVQLLESGGGLVQPGGSLRLSCAASGFTFSNAWMSWVRQAPGKGLEWVSS
ISTSSNYIYYADSVKGRFTISRDNSKNTLYLQMNSLRAEDTAVYYCARVK
KYSSGWYSNYAFDIWGQGTLVTVSS
Variable light (VL) region
(SEQ ID NO: 125)
CAGTCTGTGCTGACTCAGCCACCCTCAGCGTCTGGGACCCCCGGGCAGAG
GGTCACCATCTCCTGCTCTGGAAGCAGCTCCAGCATTGGGAATAATTTTG
TATCCTGGTATCAGCAGCTCCCAGGAACGGCCCCCAAACTCCTCATCTAT
GACAATAATAAGCGACCCTCAGGGGTCCCTGACCGATTCTCTGGCTCCAA
GTCTGGCACCTCAGCCTCCCTGGCCATCAGTGGGCTCCGGTCCGAGGATG
AGGCTGATTATTACTGTGCAGCATGGGATGACAGCCTGAATGGTTGGGTG
TTCGGCGGAGGAACCAAGCTGACGGTCCTAGGT
Variable light (VL) region
(SEQ ID NO: 126)
QSVLTQPPSASGTPGQRVTISCSGSSSSIGNNFVSWYQQLPGTAPKLLIY
DNNKRPSGVPDRFSGSKSGTSASLAISGLRSEDEADYYCAAWDDSLNGWV
FGGGTKLTVLG

In some embodiments, the human antibody comprises one or more (at least one, two, three, four, five, or six) CDRs selected from the group consisting of CSGSNTNIGKNYVS (SEQ ID NO:39), ANSNRPS (SEQ ID NO:40), CASWDASLNGWV (SEQ ID NO:41), FSNAWMSWVRQAPG (SEQ ID NO:42), SSISVGGHRTYYADSVKGR, (SEQ ID NO:43), and ARIRVGPSGGAFDY (SEQ ID NO:44). In some embodiments, the human antibody comprises a light chain variable domain comprising (i) CDR-L1 comprising sequence CSGSNTNIGKNYVS (SEQ ID NO:39); (ii) CDR-L2 comprising sequence ANSNRPS (SEQ ID NO:40); and (iii) CDR-L3 comprising sequence CASWDASLNGWV (SEQ ID NO:41). In some embodiments, the human antibody comprises a heavy chain variable domain comprising (i) CDR-H1 comprising sequence FSNAWMSWVRQAPG (SEQ ID NO:42); (ii) CDR-H2 comprising sequence SSISVGGHRTYYADSVKGR (SEQ ID NO:43); and (iii) CDR-H3 comprising sequence ARIRVGPSGGAFDY (SEQ ID NO:44). In some embodiments, the human antibody comprises a light chain variable domain comprising (i) CDR-L1 comprising sequence CSGSNTNIGKNYVS (SEQ ID NO:39); (ii) CDR-L2 comprising sequence ANSNRPS (SEQ ID NO:40); and (iii) CDR-L3 comprising sequence CASWDASLNGWV (SEQ ID NO:41) and a heavy chain variable domain comprising (i) CDR-H1 comprising sequence FSNAWMSWVRQAPG (SEQ ID NO:42); (ii) CDR-H2 comprising sequence SSISVGGHRTYYADSVKGR (SEQ ID NO:43); and (iii) CDR-H3 comprising sequence ARIRVGPSGGAFDY (SEQ ID NO:44).

In some embodiments, the human antibody comprises one or more (at least one, two, three, four, five, or six) CDRs selected from the group consisting of CSGSSSNIGNNAVN (SEQ ID NO:45), GNDRRPS (SEQ ID NO:46), CQTWGTGRGV (SEQ ID NO:47), FSDYYMSWVRQAPG (SEQ ID NO:48), SGVSWNGSRTHYADSVKGR (SEQ ID NO:49), and ARAARYSYYYYGMDV (SEQ ID NO:50). In some embodiments, the human antibody comprises a light chain variable domain comprising (i) CDR-L1 comprising sequence CSGSSSNIGNNAVN (SEQ ID NO:45); (ii) CDR-L2 comprising sequence GNDRRPS (SEQ ID NO:46); and (iii) CDR-L3 comprising sequence CQTWGTGRGV (SEQ ID NO:47). In some embodiments, the human antibody comprises a heavy chain variable domain comprising (i) CDR-H1 comprising sequence FSDYYMSWVRQAPG (SEQ ID NO:48); (ii) CDR-H2 comprising sequence SGVSWNGSRTHYADSVKGR (SEQ ID NO:49); and (iii) CDR-H3 comprising sequence ARAARYSYYYYGMDV (SEQ ID NO:50). In some embodiments, the human antibody comprises a light chain variable domain comprising (i) CDR-L1 comprising sequence CSGSSSNIGNNAVN (SEQ ID NO:45); (ii) CDR-L2 comprising sequence GNDRRPS (SEQ ID NO:46); and (iii) CDR-L3 comprising sequence CQTWGTGRGV (SEQ ID NO:47) and a heavy chain variable domain comprising (i) CDR-H1 comprising sequence FSDYYMSWVRQAPG (SEQ ID NO:48); (ii) CDR-H2 comprising sequence SGVSWNGSRTHYADSVKGR (SEQ ID NO:49); and (iii) CDR-H3 comprising sequence ARAARYSYYYYGMDV (SEQ ID NO:50).

In some embodiments, the human antibody comprises one or more (at least one, two, three, four, five, or six) CDRs selected from the group consisting of CSGSSSSIGNNFVS (SEQ ID NO:51), DNNKRPS (SEQ ID NO:52), CAAWDDSLNGWV (SEQ ID NO:53), FSNAWMSWVRQAPG (SEQ ID NO:54), SSISTSSNYIYYADSVKGR (SEQ ID NO:55), and ARVKKYSSGWYSNYAFDI (SEQ ID NO:56). In some embodiments, the human antibody comprises a light chain variable domain comprising (i) CDR-L1 comprising sequence CSGSSSSIGNNFVS (SEQ ID NO:51); (ii) CDR-L2 comprising sequence DNNKRPS (SEQ ID NO:52); and (iii) CDR-L3 comprising sequence CAAWDDSLNGWV (SEQ ID NO:53). In some embodiments, the human antibody comprises a heavy chain variable domain comprising (i) CDR-H1 comprising sequence FSNAWMSWVRQAPG (SEQ ID NO:54); (ii) CDR-H2 comprising sequence SSISTSSNYIYYADSVKGR (SEQ ID NO:55); and (iii) CDR-H3 comprising sequence ARVKKYSSGWYSNYAFDI (SEQ ID NO:56). In some embodiments, the human antibody comprises a light chain variable domain comprising (i) CDR-L1 comprising sequence CSGSSSSIGNNFVS (SEQ ID NO:51); (ii) CDR-L2 comprising sequence DNNKRPS (SEQ ID NO:52); and (iii) CDR-L3 comprising sequence CAAWDDSLNGWV (SEQ ID NO:53) and a heavy chain variable domain comprising (i) CDR-H1 comprising sequence FSNAWMSWVRQAPG (SEQ ID NO:54); (ii) CDR-H2 comprising sequence SSISTSSNYIYYADSVKGR (SEQ ID NO:55); and (iii) CDR-H3 comprising sequence ARVKKYSSGWYSNYAFDI (SEQ ID NO:56).

In some embodiments, the human antibody comprises one or more (at least one, two, three, four, five, or six) CDRs selected from the group consisting of CSGSSSNIGGESVS (SEQ ID NO:57), SNNQRPS (SEQ ID NO:58), CAAWDDSLNGWV (SEQ ID NO:59), FSSYAMSWVRQAPG (SEQ ID NO:60), SSISSSGRFIYYADSMKGR (SEQ ID NO:61), and TRLRRGSYFWAFDI (SEQ ID NO:62). In some embodiments, the human antibody comprises a light chain variable domain comprising (i) CDR-L1 comprising sequence CSGSSSNIGGESVS (SEQ ID NO:57); (ii) CDR-L2 comprising sequence SNNQRPS (SEQ ID NO:58); and (iii) CDR-L3 comprising sequence CAAWDDSLNGWV (SEQ ID NO:59). In some embodiments, the human antibody comprises a heavy chain variable domain comprising (i) CDR-H1 comprising sequence FSSYAMSWVRQAPG (SEQ ID NO:60); (ii) CDR-H2 comprising sequence SSISSSGRFIYYADSMKGR (SEQ ID NO:61); and (iii) CDR-H3 comprising sequence TRLRRGSYFWAFDI (SEQ ID NO:62). In some embodiments, the human antibody comprises a light chain variable domain comprising (i) CDR-L1 comprising sequence CSGSSSNIGGESVS (SEQ ID NO:57); (ii) CDR-L2 comprising sequence SNNQRPS (SEQ ID NO:58); and (iii) CDR-L3 comprising sequence CAAWDDSLNGWV (SEQ ID NO:59) and a heavy chain variable domain comprising (i) CDR-H1 comprising sequence FSSYAMSWVRQAPG (SEQ ID NO:60); (ii) CDR-H2 comprising sequence SSISSSGRFIYYADSMKGR (SEQ ID NO:61); and (iii) CDR-H3 comprising sequence TRLRRGSYFWAFDI (SEQ ID NO:62).

In some embodiments, the human antibody comprises one or more (at least one, two, three, four, five, or six) CDRs selected from the group consisting of CSGSSSNIGNNYVS (SEQ ID NO:127), SNNQRPS (SEQ ID NO:128), CAAWDDSLSHWL (SEQ ID NO:129), FSNAWMSWVRQVPG (SEQ ID NO:130), STLGGSGGGSTYYADSVKGR (SEQ ID NO:131), and AKLGGRSRYGRWPRQFDY (SEQ ID NO:132). In some embodiments, the human antibody comprises a light chain variable domain comprising (i) CDR-L1 comprising sequence CSGSSSNIGNNYVS (SEQ ID NO:127); (ii) CDR-L2 comprising sequence SNNQRPS (SEQ ID NO:128); and (iii) CDR-L3 comprising sequence CAAWDDSLSHWL (SEQ ID NO:129). In some embodiments, the human antibody comprises a heavy chain variable domain comprising (i) CDR-H1 comprising sequence FSNAWMSWVRQVPG (SEQ ID NO:130); (ii) CDR-H2 comprising sequence STLGGSGGGSTYYADSVKGR (SEQ ID NO:131); and (iii) CDR-H3 comprising sequence AKLGGRSRYGRWPRQFDY (SEQ ID NO:132). In some embodiments, the human antibody comprises a light chain variable domain comprising (i) CDR-L1 comprising sequence CSGSSSNIGNNYVS (SEQ ID NO:127); (ii) CDR-L2 comprising sequence SNNQRPS (SEQ ID NO:128); and (iii) CDR-L3 comprising sequence CAAWDDSLSHWL (SEQ ID NO:129) and a heavy chain variable domain comprising (i) CDR-H1 comprising sequence FSNAWMSWVRQVPG (SEQ ID NO:130); (ii) CDR-H2 comprising sequence STLGGSGGGSTYYADSVKGR (SEQ ID NO:131); and (iii) CDR-H3 comprising sequence AKLGGRSRYGRWPRQFDY (SEQ ID NO:132.

In some embodiments, the human antibody comprises one or more (at least one, two, three, four, five, or six) CDRs selected from the group consisting of CSGSSSNIGSNYVS (SEQ ID NO:133), GNYNRPS (SEQ ID NO:134), CAAWDDSLSGWV (SEQ ID NO:135), FSSYWMSWVRQAPG (SEQ ID NO:136), SSISGSGRRTYYADSVQGR (SEQ ID NO:137), and ARLVSYGSGSFGFDY (SEQ ID NO:138). In some embodiments, the human antibody comprises a light chain variable domain comprising (i) CDR-L1 comprising sequence CSGSSSNIGSNYVS (SEQ ID NO:133); (ii) CDR-L2 comprising sequence GNYNRPS (SEQ ID NO:134); and (iii) CDR-L3 comprising sequence CAAWDDSLSGWV (SEQ ID NO:135). In some embodiments, the human antibody comprises a heavy chain variable domain comprising (i) CDR-H1 comprising sequence FSSYWMSWVRQAPG (SEQ ID NO:136); (ii) CDR-H2 comprising sequence SSISGSGRRTYYADSVQGR (SEQ ID NO:137); and (iii) CDR-H3 comprising sequence ARLVSYGSGSFGFDY (SEQ ID NO:138). In some embodiments, the human antibody comprises a light chain variable domain comprising (i) CDR-L1 comprising sequence CSGSSSNIGSNYVS (SEQ ID NO:133); (ii) CDR-L2 comprising sequence GNYNRPS (SEQ ID NO:134); and (iii) CDR-L3 comprising sequence CAAWDDSLSGWV (SEQ ID NO:135) and a heavy chain variable domain comprising (i) CDR-H1 comprising sequence FSSYWMSWVRQAPG (SEQ ID NO:136); (ii) CDR-H2 comprising sequence SSISGSGRRTYYADSVQGR (SEQ ID NO:137); and (iii) CDR-H3 comprising sequence ARLVSYGSGSFGFDY (SEQ ID NO:138).

In some embodiments, the human antibody comprises one or more (at least one, two, three, four, five, or six) CDRs selected from the group consisting of CSGRSSNIGNSYVS (SEQ ID NO:139), RNNQRPS (SEQ ID NO:140), CAGWDDTLRAWV (SEQ ID NO:141), FRDYYVSWIRQAPG (SEQ ID NO:142), SSISGSGGRTYYADSVEGR (SEQ ID NO:143), and ARVSALRRPMTTVTTYWFDP (SEQ ID NO:144). In some embodiments, the human antibody comprises a light chain variable domain comprising (i) CDR-L1 comprising sequence CSGRSSNIGNSYVS (SEQ ID NO:139); (ii) CDR-L2 comprising sequence RNNQRPS (SEQ ID NO:140); and (iii) CDR-L3 comprising sequence CAGWDDTLRAWV (SEQ ID NO:141). In some embodiments, the human antibody comprises a heavy chain variable domain comprising (i) CDR-H1 comprising sequence FRDYYVSWIRQAPG (SEQ ID NO:142); (ii) CDR-H2 comprising sequence SSISGSGGRTYYADSVEGR (SEQ ID NO:143); and (iii) CDR-H3 comprising sequence ARVSALRRPMTTVTTYWFDP (SEQ ID NO:144). In some embodiments, the human antibody comprises a light chain variable domain comprising (i) CDR-L1 comprising sequence CSGRSSNIGNSYVS (SEQ ID NO:139); (ii) CDR-L2 comprising sequence RNNQRPS (SEQ ID NO:140); and (iii) CDR-L3 comprising sequence CAGWDDTLRAWV (SEQ ID NO:141) and a heavy chain variable domain comprising (i) CDR-H1 comprising sequence FRDYYVSWIRQAPG (SEQ ID NO:142); (ii) CDR-H2 comprising sequence SSISGSGGRTYYADSVEGR (SEQ ID NO:143); and (iii) CDR-H3 comprising sequence ARVSALRRPMTTVTTYWFDP (SEQ ID NO:144).

### (vi) Antibody Fragments

In some embodiments, the anti-oxidized LDL antibodies are antibody fragments. Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992) and Brennan et al., Science 229:81 (1985)). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F(ab′)₂ fragments (Carter et al., Bio/Technology 10:163-167 (1992)). According to another approach, F(ab′)₂ fragments can be isolated directly from recombinant host cell culture. Other techniques for the production of antibody fragments will be apparent to the skilled practitioner. In other embodiments, the antibody of choice is a single chain Fv fragment (scFv). See WO 93/16185; U.S. Pat. No. 5,571,894; and U.S. Pat. No. 5,587,458. The antibody fragment may also be a “linear antibody”, e.g., as described in U.S. Pat. No. 5,641,870 for example. Such linear antibody fragments may be monospecific or bispecific.

In some embodiments, fragments of the antibodies described herein are provided. In some embodiments, the antibody fragments are antigen binding fragments.

(vii) Bispecific Antibodies

In some embodiments, the anti-oxidized LDL antibodies are bispecific antibodies. Bispecific antibodies are antibodies that have binding specificities for at least two different epitopes. Exemplary bispecific antibodies may bind to two different epitopes of oxidized LDL. Other such antibodies may bind the oxidized LDL and further bind a second different oxidized LDL. Alternatively, an anti-oxidized LDL binding arm may be combined with an arm that binds to a triggering molecule on a leukocyte such as a T-cell receptor molecule (e.g. CD2 or CD3), or Fc receptors for IgG (FcγR), such as FcγRI (CD64), FcγRII (CD32) and FcγRIII (CD16) so as to focus cellular defense mechanisms to the cell. Bispecific antibodies can be prepared as full length antibodies or antibody fragments (e.g. F(ab′)₂ bispecific antibodies).

Methods for making bispecific antibodies are known in the art. Traditional production of full length bispecific antibodies is based on the coexpression of two immunoglobulin heavy chain-light chain pairs, where the two chains have different specificities (Millstein et al., Nature 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J., 10:3655-3659 (1991).

According to a different approach, antibody variable domains with the desired binding specificities (antibody-antigen combining sites) are fused to immunoglobulin constant domain sequences. In some embodiments, the fusion is with an immunoglobulin heavy chain constant domain, comprising at least part of the hinge, CH2, and CH3 regions. In some embodiments, the first heavy chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In some embodiments of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology 121:210 (1986).

According to another approach described in U.S. Pat. No. 5,731,168, the interface between a pair of antibody molecules can be engineered to maximize the percentage of heterodimers that are recovered from recombinant cell culture. In some embodiments, the interface comprises at least a part of the C_{H}3 domain of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science 229: 81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy chain variable domain (V_{H}) connected to a light chain variable domain (V_{L}) by a linker that is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber et al., J. Immunol. 152:5368 (1994).

Antibodies with more than two valencies are contemplated. For example, trispecific antibodies can be prepared. Tutt et al., J. Immunol. 147: 60 (1991).

(viii) Multivalent Antibodies

In some embodiments, the anti-oxidized LDL antibodies are multivalent antibodies. A multivalent antibody may be internalized (and/or catabolized) faster than a bivalent antibody by a cell expressing an antigen to which the antibodies bind. The antibodies provided herein can be multivalent antibodies (which are other than of the IgM class) with three or more antigen binding sites (e.g., tetravalent antibodies), which can be readily produced by recombinant expression of nucleic acid encoding the polypeptide chains of the antibody. The multivalent antibody can comprise a dimerization domain and three or more antigen binding sites. The preferred dimerization domain comprises (or consists of) an Fc region or a hinge region. In this scenario, the antibody will comprise an Fc region and three or more antigen binding sites amino-terminal to the Fc region. The preferred multivalent antibody herein comprises (or consists of) three to about eight, but preferably four, antigen binding sites. The multivalent antibody comprises at least one polypeptide chain (and preferably two polypeptide chains), wherein the polypeptide chain(s) comprise two or more variable domains. For instance, the polypeptide chain(s) may comprise VD1-(X1)ₙ-VD2-(X2)ₙ-Fc, wherein VD1 is a first variable domain, VD2 is a second variable domain, Fc is one polypeptide chain of an Fc region, X1 and X2 represent an amino acid or polypeptide, and n is 0 or 1. For instance, the polypeptide chain(s) may comprise: VH-CH1-flexible linker-VH-CH1-Fc region chain; or VH-CH1-VH-CH1-Fc region chain. The multivalent antibody herein preferably further comprises at least two (and preferably four) light chain variable domain polypeptides. The multivalent antibody herein may, for instance, comprise from about two to about eight light chain variable domain polypeptides. The light chain variable domain polypeptides contemplated here comprise a light chain variable domain and, optionally, further comprise a CL domain

### (ix) Other Amino Acid Sequence Modifications

Amino acid sequence modification(s) of the oxidized LDL binding antibodies described herein are contemplated. For example, it may be desirable to improve the binding affinity and/or other biological properties of the antibody. Amino acid sequence variants of the anti-oxidized LDL antibody are prepared by introducing appropriate nucleotide changes into the anti-oxidized LDL antibody nucleic acid, or by peptide synthesis. Such modifications include, for example, deletions from, and/or insertions into and/or substitutions of, residues within the amino acid sequences of the anti-oxidized LDL antibody. Any combination of deletion, insertion, and substitution is made to arrive at the final construct, provided that the final construct possesses the desired characteristics. The amino acid changes also may alter post-translational processes of the anti-oxidized LDL antibody, such as changing the number or position of glycosylation sites.

A useful method for identification of certain residues or regions of the anti-oxidized LDL antibody that are preferred locations for mutagenesis is called “alanine scanning mutagenesis” as described by Cunningham and Wells, Science 244:1081-1085 (1989). Here, a residue or group of target residues are identified (e.g., charged residues such as Arg, Asp, His, Lys, and Glu) and replaced by a neutral or negatively charged amino acid (most preferably Alanine or Polyalanine) to affect the interaction of the amino acids with oxidized LDL antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the expressed anti-oxidized LDL antibody variants are screened for the desired activity.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an anti-oxidized LDL antibody with an N-terminal methionyl residue or the antibody fused to a cytotoxic polypeptide. Other insertional variants of the anti-oxidized LDL antibody molecule include the fusion to the N- or C-terminus of the anti-oxidized LDL antibody to an enzyme (e.g. for ADEPT) or a polypeptide which increases the serum half-life of the antibody.

Another type of variant is an amino acid substitution variant. These variants have at least one amino acid residue in the anti-oxidized LDL antibody molecule replaced by a different residue. The sites of greatest interest for substitutional mutagenesis include the hypervariable regions, but FR alterations are also contemplated. Conservative substitutions are shown in the Table below under the heading of “preferred substitutions”. If such substitutions result in a change in biological activity, then more substantial changes, denominated “exemplary substitutions” in the Table, or as further described below in reference to amino acid classes, may be introduced and the products screened.

TABLE 3

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Amino acids may be grouped according to similarities in the properties of their side chains (in A. L. Lehninger, in Biochemistry, second ed., pp. 73-75, Worth Publishers, New York (1975)):

(1) non-polar: Ala (A), Val (V), Leu (L), Ile (I), Pro (P), Phe (F), Trp (W), Met (M)

(2) uncharged polar: Gly (G), Ser (S), Thr (T), Cys (C), Tyr (Y), Asn (N), Gln (Q)

(3) acidic: Asp (D), Glu (E)

(4) basic: Lys (K), Arg (R), His (H)

Alternatively, naturally occurring residues may be divided into groups based on common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

Non-conservative substitutions will entail exchanging a member of one of these classes for another class.

Any cysteine residue not involved in maintaining the proper conformation of the anti-oxidized LDL antibody also may be substituted, generally with serine, to improve the oxidative stability of the molecule and prevent aberrant crosslinking. Conversely, cysteine bond(s) may be added to the antibody to improve its stability (particularly where the antibody is an antibody fragment such as an Fv fragment).

A particularly preferred type of substitutional variant involves substituting one or more hypervariable region residues of a parent antibody (e.g., a humanized antibody). Generally, the resulting variant(s) selected for further development will have improved biological properties relative to the parent antibody from which they are generated. A convenient way for generating such substitutional variants involves affinity maturation using phage display. Briefly, several hypervariable region sites (e.g., 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g., binding affinity) as herein disclosed. In order to identify candidate hypervariable region sites for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the antibody and oxidized LDL. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Another type of amino acid variant of the antibody alters the original glycosylation pattern of the antibody. The antibodies may comprise non-amino acid moieties. For example, the antibodies may be glycosylated. Such glycosylation may occur naturally during expression of the antibodies in the host cell or host organism, or may be a deliberate modification arising from human intervention. By altering is meant deleting one or more carbohydrate moieties found in the antibody, and/or adding one or more glycosylation sites that are not present in the antibody.

Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in an antibody amino acid sequence creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Nucleic acid molecules encoding amino acid sequence variants of the anti-oxidized LDL antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the anti-oxidized LDL antibody.

It may be desirable to modify the antibody provided herein with respect to effector function, e.g., so as to enhance antigen-dependent cell-mediated cyotoxicity (ADCC) and/or complement dependent cytotoxicity (CDC) of the antibody. This may be achieved by introducing one or more amino acid substitutions in an Fc region of the antibody. Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med. 176:1191-1195 (1992) and Shopes, B. J., Immunol. 148:2918-2922 (1992). Homodimeric antibodies with enhanced anti-tumor activity may also be prepared using heterobifunctional cross-linkers as described in Wolff et al., Cancer Research 53:2560-2565 (1993). Alternatively, an antibody can be engineered which has dual Fc regions and may thereby have enhanced complement mediated lysis and ADCC capabilities. See Stevenson et al., Anti-Cancer Drug Design 3:219-230 (1989).

For increasing serum half the serum half life of the antibody, amino acid alterations can be made in the antibody as described in U.S. 2006/0067930, which is hereby incorporated by reference in its entirety.

### (x) Other Antibody Modifications

Other modifications of the antibody are contemplated herein. For example, the antibody may be linked to one of a variety of nonproteinaceous polymers, e.g., polyethylene glycol, polypropylene glycol, polyoxyalkylenes, or copolymers of polyethylene glycol and polypropylene glycol. The antibody also may be entrapped in microcapsules prepared, for example, by coacervation techniques or by interfacial polymerization (for example, hydroxymethylcellulose or gelatin-microcapsules and poly-(methylmethacylate) microcapsules, respectively), in colloidal drug delivery systems (for example, liposomes, albumin microspheres, microemulsions, nano-particles and nanocapsules), or in macroemulsions. Such techniques are disclosed in Remington's Pharmaceutical Sciences, 16th edition, Oslo, A., Ed., (1980).

Additionally or alternatively the humanized antibodies may be subjected to other chemical modification. One such desirable modification is addition of one or more polyethylene glycol (PEG) moieties. Pegylation has been shown to increase significantly the half-life of various antibody fragments in vivo (Chapman 2002 Adv. Drug Delivery Rev. 54, 531-545). However, random Pegylation of antibody fragments can have highly detrimental effects on the binding affinity of the fragment for the antigen. In order to avoid this it is desirable that Pegylation is restricted to specific, targeted residues of the humanized antibodies (see Knight et al, 2004 Platelets 15, 409-418 and Chapman, supra).

(xi) Screening for Antibodies with Desired Properties

To screen for antibodies which bind to an epitope on oxidized LDL bound by an antibody of interest, a routine cross-blocking assay such as that described in Antibodies, A Laboratory Manual, Cold Spring Harbor Laboratory, Ed Harlow and David Lane (1988), can be performed. This assay can be used to determine if a test antibody binds the same site or epitope as an anti-oxidized LDL antibody provided herein. Alternatively, or additionally, epitope mapping can be performed by methods known in the art. For example, the antibody sequence can be mutagenized such as by alanine scanning, to identify contact residues. The mutant antibody is initially tested for binding with polyclonal antibody to ensure proper folding. In a different method, peptides corresponding to different regions of oxidized LDL can be used in competition assays with the test antibodies or with a test antibody and an antibody with a characterized or known epitope.

### III. Obtaining Antibodies for Use in the Methods and Uses

The antibodies used in the methods and uses described herein may be obtained using methods well-known in the art, including recombinant methods. The following sections provide guidance regarding these methods.

### (i) Polynucleotides

“Polynucleotide,” or “nucleic acid,” as used interchangeably herein, refer to polymers of nucleotides of any length, and include DNA and RNA.

Polynucleotides may encode any of the anti-oxidized LDL antibodies described herein. For example, the polynucleotide may encode an entire immunoglobulin molecule chain, such as a light chain or a heavy chain. A complete heavy chain includes not only a heavy chain variable region (V_{H}) but also a heavy chain constant region (C_{H}), which typically will comprise three constant domains: C_{H}1, C_{H}2 and C_{H}3; and a “hinge” region. In some situations, the presence of a constant region is desirable.

The polynucleotide may encode a variable light chain and/or a variable heavy chain. In some embodiments, the polynucleotide comprises a light chain variable domain or heavy chain variable domain selected from Table 2. In some embodiments, the polynucleotide comprises a heavy chain variable domain selected from the group consisting of SEQ ID NO:63, SEQ ID NO:67, SEQ ID NO:71, SEQ ID NO:75, SEQ ID NO:79, SEQ ID NO:83, SEQ ID NO:87, SEQ ID NO:91, SEQ ID NO:95, SEQ ID NO:99, SEQ ID NO:103, SEQ ID NO:107, SEQ ID NO:111, SEQ ID NO:115, SEQ ID NO:119, and SEQ ID NO:123. In some embodiments, polynucleotide comprises a light chain variable domain selected from the group consisting of SEQ ID NO:65, SEQ ID NO:69, SEQ ID NO:73, SEQ ID NO:77, SEQ ID NO:81, SEQ ID NO:85, SEQ ID NO:89, SEQ ID NO:93, SEQ ID NO:97, SEQ ID NO:101, SEQ ID NO:105, SEQ ID NO:109, SEQ ID NO:113, SEQ ID NO:117, SEQ ID NO:121, and SEQ ID NO:125. In some embodiments, the polynucleotide comprises a heavy chain variable domain comprising SEQ ID NO:103 and a light chain variable domain comprising SEQ ID NO:105. In some embodiments, the polynucleotide comprises a heavy chain variable domain comprising SEQ ID NO:67 and a light chain variable domain comprising SEQ ID NO:69.

Other polypeptides which may be encoded by the polynucleotide include antigen-binding antibody fragments such as single domain antibodies (“dAbs”), Fv, scFv, Fab′ and F(ab)₂ and “minibodies”. Minibodies are (typically) bivalent antibody fragments from which the C_{H}1 and C_{K} or C_{L} domain has been excised. As minibodies are smaller than conventional antibodies they should achieve better tissue penetration in clinical/diagnostic use, but being bivalent they should retain higher binding affinity than monovalent antibody fragments, such as dAbs. Accordingly, unless the context dictates otherwise, the term “antibody” as used herein encompasses not only whole antibody molecules but also antigen-binding antibody fragments of the type discussed above.

Whilst the encoded polypeptide will typically have CDR sequences identical or substantially identical to those of IEIA8, IEI-G8, IEI-D8, KTT-D6, 1-B12, 1-C07, 1-C12, 1-G10, 2-F07, 2-F09, 4-A02, IEI-E3, 2D03, LDO-D4, and/or KTT-B8. The polynucleotide will thus preferably encode a polypeptide having a heavy and/or light chain variable region as described herein relative to the heavy and/or light chain (as appropriate) of IEIA8, IEI-G8, IEI-D8, KTT-D6, 1-B12, 1-C07, 1-C12, 1-G10, 2-F07, 2-F09, 4-A02, IEI-E3, 2D03, LDO-D4, and/or KTT-B8. If the encoded polypeptide comprises a partial or complete heavy and/or light chain constant region, this too is advantageously of human origin.

In some embodiments, at least one of the framework regions of the encoded polypeptide, and most preferably each of the framework regions, will comprise amino acid substitutions relative to the human acceptor so as to become more similar to those of IEIA8, IEI-G8, IEI-D8, KTT-D6, 1-B12, 1-C07, 1-C12, 1-G10, 2-F07, 2-F09, 4-A02, IEI-E3, 2D03, LDO-D4, and/or KTT-B8, so as to increase the binding activity of the humanized antibody.

In some embodiments, each framework region present in the encoded polypeptide will comprise at least one amino acid substitution relative to the corresponding human acceptor framework. Thus, for example, the framework regions may comprise, in total, three, four, five, six, seven, eight, nine, ten, eleven, twelve, thirteen, fourteen or fifteen amino acid substitutions relative to the acceptor framework regions. Advantageously, the mutations are backmutations to match the residues present at the equivalent positions in the IEIA8, IEI-G8, IEI-D8, KTT-D6, 1-B12, 1-C07, 1-C12, 1-G10, 2-F07, 2-F09, 4-A02, IEI-E3, 2D03, LDO-D4, and/or KTT-B8 framework. In some embodiments, six backmutations are made in the heavy chain and one in the light chain.

Suitably, the polynucleotide described herein may be isolated and/or purified. In some embodiments, the polynucleotide is an isolated polynucleotide.

The term “isolated polynucleotide” is intended to indicate that the molecule is removed or separated from its normal or natural environment or has been produced in such a way that it is not present in its normal or natural environment. In some embodiments, the polynucleotides are purified polynucleotides. The term purified is intended to indicate that at least some contaminating molecules or substances have been removed.

Suitably, the polynucleotide is substantially purified, such that the relevant polynucleotide constitutes the dominant (i.e., most abundant) polynucleotide present in a composition.

Recombinant nucleic acids comprising an insert coding for a heavy chain variable domain and/or for a light chain variable domain may be used in the methods as described herein. By definition such nucleic acids comprise coding single stranded nucleic acids, double stranded nucleic acids consisting of said coding nucleic acids and of complementary nucleic acids thereto, or these complementary (single stranded) nucleic acids themselves.

Modification(s) may also be made outside the heavy chain variable domain and/or of the light chain variable domain of the anti-oxidized LDL antibody. Such a mutant nucleic acid may be a silent mutant wherein one or more nucleotides are replaced by other nucleotides with the new codons coding for the same amino acid(s). Such a mutant sequence may be a degenerate sequence. Degenerate sequences are degenerated within the meaning of the genetic code in that an unlimited number of nucleotides are replaced by other nucleotides without resulting in a change of the amino acid sequence originally encoded. Such degenerated sequences may be useful due to their different restriction sites and/or frequency of particular codons which are preferred by the specific host, particularly yeast, bacterial or mammalian cells, to obtain an optimal expression of the heavy chain variable domain and/or the light chain variable domain.

Provided herein is also the use of sequences having a degree of sequence identity or sequence homology with amino acid sequence(s) of a polypeptide having the specific properties defined herein or of any nucleotide sequence encoding such a polypeptide (hereinafter referred to as a “homologous sequence(s)”). Here, the term “homologue” means an entity having a certain homology with the subject amino acid sequences and the subject nucleotide sequences. Here, the term “homology” can be equated with “identity”.

In some embodiments, homologous amino acid sequence and/or nucleotide sequence should provide and/or encode a polypeptide which retains the functional activity and/or enhances the activity of the antibody. In some embodiments, homologous sequence is taken to include an amino acid sequence which may be at least 75, 85, or 90% identical, preferably at least 95 or 98% identical to the subject sequence. Typically, the homologues will comprise the same active sites etc. as the subject amino acid sequence. Although homology can also be considered in terms of similarity (i.e., amino acid residues having similar chemical properties/functions). In some embodiments, it is preferred to express homology in terms of sequence identity.

In the present context, a homologous sequence is taken to include a nucleotide sequence which may be at least 75, 85, or 90% identical, preferably at least 95 or 98% identical to a nucleotide sequence encoding a polypeptide described herein (the subject sequence). Typically, the homologues will comprise the same sequences that code for the active sites etc. as the subject sequence. Although homology can also be considered in terms of similarity (i.e., amino acid residues having similar chemical properties/functions). In some embodiments, it is preferred to express homology in terms of sequence identity.

### (ii) Expression of Recombinant Antibodies

The description below relates primarily to production of polypeptides by culturing cells transformed or transfected with a vector containing polypeptide-encoding polynucleotides. It is, of course, contemplated that alternative methods, which are well-known in the art, may be employed to prepare polypeptides (e.g., solid-phase techniques or in vitro protein synthesis).

Polynucleotides as described herein may be inserted into an expression vector(s) for production of antibodies. Polynucleotide encoding light and heavy chain variable regions as described herein are optionally linked to constant regions, and inserted into an expression vector(s). The light and heavy chains can be cloned in the same or different expression vectors. The DNA segments encoding immunoglobulin chains are operably linked to control sequences in the expression vector(s) that ensure the expression of immunoglobulin polypeptides. Expression control sequences include, but are not limited to, promoters (e.g., naturally-associated or heterologous promoters), signal sequences, enhancer elements, and transcription termination sequences.

Suitably, the expression control sequences are eukaryotic promoter systems in vectors capable of transforming or transfecting eukaryotic host cells (e.g., COS cells—such as COS 7 cells—or CHO cells). Once the vector has been incorporated into the appropriate host, the host is maintained under conditions suitable for high level expression of the nucleotide sequences, and the collection and purification of the cross-reacting antibodies.

These expression vectors are typically replicable in the host organisms either as episomes or as an integral part of the host chromosomal DNA.

Selection Gene Component—Commonly, expression vectors contain selection markers (e.g., ampicillin-resistance, hygromycin-resistance, tetracycline resistance, kanamycin resistance or neomycin resistance) to permit detection of those cells transformed with the desired DNA sequences (see, e.g., Itakura et al., U.S. Pat. No. 4,704,362). In some embodiments, selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

One example of a selection scheme utilizes a drug to arrest growth of a host cell. Those cells that are successfully transformed with a heterologous gene produce a protein conferring drug resistance and thus survive the selection regimen. Examples of such dominant selection use the drugs neomycin, mycophenolic acid and hygromycin.

Another example of suitable selectable markers for mammalian cells are those that enable the identification of cells competent to take up the nucleic acid encoding anti-oxidized LDL antibodies described herein, such as DHFR, thymidine kinase, metallothionein-I and -III, preferably primate metallothionein genes, adenosine deaminase, ornithine decarboxylase, etc.

For example, cells transformed with the DHFR selection gene are first identified by culturing all of the transformants in a culture medium that contains methotrexate (Mtx), a competitive antagonist of DHFR. An appropriate host cell when wild-type DHFR is employed is the Chinese hamster ovary (CHO) cell line deficient in DHFR activity (e.g., ATCC CRL-9096).

Alternatively, host cells (particularly wild-type hosts that contain endogenous DHFR) transformed or co-transformed with DNA sequences encoding an antibody described herein, wild-type DHFR protein, and another selectable marker such as aminoglycoside 3′-phosphotransferase (APH) can be selected by cell growth in medium containing a selection agent for the selectable marker such as an aminoglycosidic antibiotic, e.g., kanamycin, neomycin, or G418. See U.S. Pat. No. 4,965,199.

A suitable selection gene for use in yeast is the trp1 gene present in the yeast plasmid YRp7 (Stinchcomb et al., Nature 282:39 (1979)). The trp1 gene provides a selection marker for a mutant strain of yeast lacking the ability to grow in tryptophan, for example, ATCC No. 44076 or PEP4-1. Jones, Genetics 85:12 (1977). The presence of the trp1 lesion in the yeast host cell genome then provides an effective environment for detecting transformation by growth in the absence of tryptophan. Similarly, Leu2-deficient yeast strains (ATCC 20,622 or 38,626) are complemented by known plasmids bearing the Leu2 gene.

In addition, vectors derived from the 1.6 um circular plasmid pKD1 can be used for transformation of Kluyveromyces yeasts. Alternatively, an expression system for large-scale production of recombinant calf chymosin was reported for K lactis. Van den Berg, Bio/Technology 8:135 (1990). Stable multi-copy expression vectors for secretion of mature recombinant human serum albumin by industrial strains of Kluyveromyces have also been disclosed. Fleer et al., Bio/Technology 9:968-975 (1991).

Signal Sequence Component—The anti-oxidized LDL antibodies may be produced recombinantly not only directly, but also as a fusion polypeptide with a heterologous polypeptide, which is preferably a signal sequence or other polypeptide having a specific cleavage site at the N-terminus of the mature protein or polypeptide. The heterologous signal sequence selected preferably is one that is recognized and processed (i.e., cleaved by a signal peptidase) by the host cell. A signal sequence can be substituted with a prokaryotic signal sequence selected, for example, from the group of the alkaline phosphatase, penicillinase, 1 pp, or heat-stable enterotoxin II leaders. For yeast secretion the native signal sequence may be substituted by, e.g., the yeast invertase leader, a factor leader (including Saccharomyces and Kluyveromyces α-factor leaders), or acid phosphatase leader, the C. albicans glucoamylase leader, or the signal described in WO 90/13646. In mammalian cell expression, mammalian signal sequences as well as viral secretory leaders, for example, the herpes simplex gD signal, are available.

The DNA for such precursor region is ligated in reading frame to DNA encoding the anti-oxidized LDL antibodies described herein.

Origin of Replication—Both expression and cloning vectors contain a nucleic acid sequence that enables the vector to replicate in one or more selected host cells. Generally, in cloning vectors this sequence is one that enables the vector to replicate independently of the host chromosomal DNA, and includes origins of replication or autonomously replicating sequences. Such sequences are well known for a variety of bacteria, yeast, and viruses. The origin of replication from the plasmid pBR322 is suitable for most Gram-negative bacteria, the 2μ plasmid origin is suitable for yeast, and various viral origins (SV40, polyoma, adenovirus, VSV or BPV) are useful for cloning vectors in mammalian cells. Generally, the origin of replication component is not needed for mammalian expression vectors (the SV40 origin may typically be used only because it contains the early promoter).

Promoter Component—Expression and cloning vectors usually contain a promoter that is recognized by the host organism and is operably linked to the nucleic acid encoding an anti-oxidized LDL antibody. Promoters suitable for use with prokaryotic hosts include the phoA promoter, β-lactamase and lactose promoter systems, alkaline phosphatase promoter, a tryptophan (trp) promoter system, and hybrid promoters such as the tac promoter. However, other known bacterial promoters are suitable. Promoters for use in bacterial systems also will contain a Shine-Dalgarno (S.D.) sequence operably linked to the DNA encoding the anti-oxidized LDL antibody.

Promoter sequences are known for eukaryotes. Virtually all eukaryotic genes have an AT-rich region located approximately 25 to 30 bases upstream from the site where transcription is initiated. Another sequence found 70 to 80 bases upstream from the start of transcription of many genes is a CNCAAT region where N may be any nucleotide. At the 3′ end of most eukaryotic genes is an AATAAA sequence that may be the signal for addition of the poly A tail to the 3′ end of the coding sequence. All of these sequences are suitably inserted into eukaryotic expression vectors.

Examples of suitable promoter sequences for use with yeast hosts include the promoters for 3-phosphoglycerate kinase or other glycolytic enzymes, such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657. Yeast enhancers also are advantageously used with yeast promoters.

The transcription of an anti-oxidized LDL antibody described herein from vectors in mammalian host cells is controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and most preferably Simian Virus 40 (SV40), from heterologous mammalian promoters, e.g., the actin promoter or an immunoglobulin promoter, from heat-shock promoters, provided such promoters are compatible with the host cell systems.

The early and late promoters of the SV40 virus are conveniently obtained as an SV40 restriction fragment that also contains the SV40 viral origin of replication. The immediate early promoter of the human cytomegalovirus is conveniently obtained as a HindIII E restriction fragment. A system for expressing DNA in mammalian hosts using the bovine papilloma virus as a vector is disclosed in U.S. Pat. No. 4,419,446. A modification of this system is described in U.S. Pat. No. 4,601,978. See also Reyes et al., Nature 297:598-601 (1982) on expression of human β-interferon cDNA in mouse cells under the control of a thymidine kinase promoter from herpes simplex virus. Alternatively, the Rous Sarcoma Virus long terminal repeat can be used as the promoter.

Enhancer Element Component—Transcription of a DNA encoding the anti-oxidized LDL antibody described herein by higher eukaryotes is often increased by inserting an enhancer sequence into the vector. Many enhancer sequences are now known from mammalian genes (globin, elastase, albumin, .alpha.-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. See also Yaniv, Nature 297:17-18 (1982) on enhancing elements for activation of eukaryotic promoters. The enhancer may be spliced into the vector at a position 5′ or 3′ to the anti-oxidized LDL antibody-encoding sequence, but is preferably located at a site 5′ from the promoter.

Transcription Termination Component—Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. One useful transcription termination component is the bovine growth hormone polyadenylation region. See WO94/11026 and the expression vector disclosed therein.

The vectors containing the polynucleotide sequences (e.g., the variable heavy and/or variable light chain encoding sequences and optional expression control sequences) can be transferred into a host cell by well-known methods, which vary depending on the type of cellular host. For example, calcium chloride transfection is commonly utilized for prokaryotic cells, whereas calcium phosphate treatment, electroporation, lipofection, biolistics or viral-based transfection may be used for other cellular hosts. (See generally Sambrook et al., Molecular Cloning: A Laboratory Manual (Cold Spring Harbor Press, 2nd ed., 1989). Other methods used to transform mammalian cells include the use of polybrene, protoplast fusion, liposomes, electroporation, and microinjection (see generally, Sambrook et al., supra). For production of transgenic animals, transgenes can be microinjected into fertilized oocytes, or can be incorporated into the genome of embryonic stem cells, and the nuclei of such cells transferred into enucleated oocytes.

When heavy and light chains are cloned on separate expression vectors, the vectors are co-transfected to obtain expression and assembly of intact immunoglobulins. Once expressed, the whole antibodies, their dimers, individual light and heavy chains, or other immunoglobulin forms can be purified according to standard procedures of the art, including ammonium sulfate precipitation, affinity columns, column chromatography, HPLC purification, gel electrophoresis and the like (see generally Scopes, Protein Purification (Springer-Verlag, N.Y., (1982)). Substantially pure immunoglobulins of at least about 90 to 95% homogeneity are preferred, and 98 to 99% or more homogeneity is most preferred, for pharmaceutical uses.

(iii) Constructs

Typically the construct will be an expression vector allowing expression, in a suitable host, of the polypeptide(s) encoded by the polynucleotide. The construct may comprise, for example, one or more of the following: a promoter active in the host; one or more regulatory sequences, such as enhancers; an origin of replication; and a marker, preferably a selectable marker. The host may be a eukaryotic or prokaryotic host, although eukaryotic (and especially mammalian) hosts may be preferred. The selection of suitable promoters will obviously depend to some extent on the host cell used, but may include promoters from human viruses such as HSV, SV40, RSV and the like. Numerous promoters are known to those skilled in the art.

The construct may comprise a polynucleotide which encodes a polypeptide comprising three light chain hypervariable loops or three heavy chain hypervariable loops. Alternatively the polynucleotide may encode a polypeptide comprising three heavy chain hypervariable loops and three light chain hypervariable loops joined by a suitably flexible linker of appropriate length. Another possibility is that a single construct may comprise a polynucleotide encoding two separate polypeptides—one comprising the light chain loops and one comprising the heavy chain loops. The separate polypeptides may be independently expressed or may form part of a single common operon.

The construct may comprise one or more regulatory features, such as an enhancer, an origin of replication, and one or more markers (selectable or otherwise). The construct may take the form of a plasmid, a yeast artificial chromosome, a yeast mini-chromosome, or be integrated into all or part of the genome of a virus, especially an attenuated virus or similar which is non-pathogenic for humans.

The construct may be conveniently formulated for safe administration to a mammalian, preferably human, subject. Typically, they will be provided in a plurality of aliquots, each aliquot containing sufficient construct for effective immunization of at least one normal adult human subject.

The construct may be provided in liquid or solid form, preferably as a freeze-dried powder which, typically, is rehydrated with a sterile aqueous liquid prior to use.

The construct may be formulated with an adjuvant or other component which has the effect of increasing the immune response of the subject (e.g., as measured by specific antibody titer) in response to administration of the construct.

### (iv) Vectors

The term “vector” includes expression vectors and transformation vectors and shuttle vectors.

The term “expression vector” means a construct capable of in vivo or in vitro expression.

The term “transformation vector” means a construct capable of being transferred from one entity to another entity—which may be of the species or may be of a different species. If the construct is capable of being transferred from one species to another—such as from an Escherichia coli plasmid to a bacterium, such as of the genus Bacillus, then the transformation vector is sometimes called a “shuttle vector”. It may even be a construct capable of being transferred from an E. coli plasmid to an Agrobacterium to a plant.

Vectors may be transformed into a suitable host cell as described below to provide for expression of a polypeptide encompassed in the present invention. Thus, in a further aspect the invention provides a process for preparing polypeptides for use in the present invention which comprises cultivating a host cell transformed or transfected with an expression vector as described above under conditions to provide for expression by the vector of a coding sequence encoding the polypeptides, and recovering the expressed polypeptides.

The vectors may be for example, plasmid, virus or phage vectors provided with an origin of replication, optionally a promoter for the expression of the said polynucleotide and optionally a regulator of the promoter.

Vectors may contain one or more selectable marker genes which are well known in the art.

### (v) Host Cells

The host cell may be a bacterium, a yeast or other fungal cell, insect cell, a plant cell, or a mammalian cell, for example.

The invention also provides a transgenic multicellular host organism which has been genetically manipulated so as to produce a polypeptide in accordance with the invention. The organism may be, for example, a transgenic mammalian organism (e.g., a transgenic goat or mouse line).

E. coli is one prokaryotic host that may be of use. Other microbial hosts include bacilli, such as Bacillus subtilis, and other enterobacteriaceae, such as Salmonella, Serratia, and various Pseudomonas species. In these prokaryotic hosts, one can make expression vectors, which will typically contain expression control sequences compatible with the host cell (e.g., an origin of replication). In addition, any number of a variety of well-known promoters will be present, such as the lactose promoter system, a tryptophan (trp) promoter system, a beta-lactamase promoter system, or a promoter system from phage lambda. The promoters will typically control expression, optionally with an operator sequence, and have ribosome binding site sequences and the like, for initiating and completing transcription and translation.

Other microbes, such as yeast, may be used for expression. Saccharomyces is a preferred yeast host, with suitable vectors having expression control sequences (e.g., promoters), an origin of replication, termination sequences and the like as desired. Typical promoters include 3-phosphoglycerate kinase and other glycolytic enzymes. Inducible yeast promoters include, among others, promoters from alcohol dehydrogenase, isocytochrome C, and enzymes responsible for maltose and galactose utilization.

In addition to microorganisms, mammalian tissue cell culture may also be used to express and produce the humanized antibodies as described herein and in some instances are preferred (See Winnacker, From Genes to Clones, VCH Publishers, N.Y., N.Y. (1987). For some embodiments, eukaryotic cells (e.g., COS7 cells) may be preferred, because a number of suitable host cell lines capable of secreting heterologous proteins (e.g., intact immunoglobulins) have been developed in the art, and include CHO cell lines, various Cos cell lines, HeLa cells, preferably, myeloma cell lines, or transformed B-cells or hybridomas.

In some embodiments, the host cell is a vertebrate host cell. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR(CHO, Urlaub et al., Proc. Natl. Acad. Sci. USA 77:4216 (1980)) or CHO-DP-12 line; mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2).

Alternatively, antibody-coding sequences can be incorporated into transgenes for introduction into the genome of a transgenic animal and subsequent expression in the milk of the transgenic animal (see, e.g., U.S. Pat. Nos. 5,741,957; 5,304,489; and 5,849,992). Suitable transgenes include coding sequences for light and/or heavy chains in operable linkage with a promoter and enhancer from a mammary gland specific gene, such as casein or beta lactoglobulin.

Alternatively, the antibodies described herein can be produced in transgenic plants (e.g., tobacco, maize, soybean and alfalfa). Improved ‘plantibody’ vectors (Hendy et al. (1999) J. Immunol. Methods 231:137-146) and purification strategies coupled with an increase in transformable crop species render such methods a practical and efficient means of producing recombinant immunoglobulins not only for human and animal therapy, but for industrial applications as well (e.g., catalytic antibodies). Moreover, plant produced antibodies have been shown to be safe and effective and avoid the use of animal-derived materials. Further, the differences in glycosylation patterns of plant and mammalian cell-produced antibodies have little or no effect on antigen binding or specificity. In addition, no evidence of toxicity or HAMA has been observed in patients receiving topical oral application of a plant-derived secretory dimeric IgA antibody (see Larrick et al. (1998) Res. Immunol. 149:603-608).

Full length antibody, antibody fragments, and antibody fusion proteins can be produced in bacteria, in particular when glycosylation and Fc effector function are not needed, such as when the therapeutic antibody is conjugated to a cytotoxic agent (e.g., a toxin) and the immunoconjugate by itself shows effectiveness in tumor cell destruction. Full length antibodies have greater half life in circulation. Production in E. coli is faster and more cost efficient. For expression of antibody fragments and polypeptides in bacteria, see, e.g., U.S. Pat. Nos. 5,648,237; 5,789,199; and 5,840,523, which describes translation initiation region (TIR) and signal sequences for optimizing expression and secretion, these patents incorporated herein by reference. After expression, the antibody is isolated from the E. coli cell paste in a soluble fraction and can be purified through, e.g., a protein A or G column depending on the isotype. Final purification can be carried out similar to the process for purifying antibody expressed e.g., in CHO cells.

Suitable host cells for the expression of glycosylated anti-oxidized LDL antibodies described herein are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells.

### (vi) Purification of Antibody

When using recombinant techniques, the antibody can be produced intracellularly, in the periplasmic space, or directly secreted into the medium. If the antibody is produced intracellularly, as a first step, the particulate debris, either host cells or lysed fragments, are removed, for example, by centrifugation or ultrafiltration. Carter et al., Bio/Technology 10: 163-167 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxylapatite chromatography, gel electrophoresis, dialysis, and affinity chromatography, with affinity chromatography being the preferred purification technique. The suitability of protein A as an affinity ligand depends on the species and isotype of any immunoglobulin Fc domain that is present in the antibody. Protein A can be used to purify antibodies that are based on human γ1, γ2, or γ4 heavy chains (Lindmark et al., J. Immunol. Meth. 62:1-13 (1983)). Protein G is recommended for all mouse isotypes and for human γ3 (Guss et al., EMBO J. 5:15671575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a C_{H3} domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g., from about 0-0.25M salt).

### IV. Pharmaceutical Formulations

Provided herein are pharmaceutical formulations comprising an anti-oxidized LDL antibody for use in the methods and uses described herein. Therapeutic formulations of the antibodies used in accordance with the present invention are prepared for storage by mixing an antibody having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

In some embodiments, the pharmaceutical formulations comprising the anti-oxidized LDL antibodies are lyophilized. Such lyophilized formulations may be reconstituted with a suitable diluent to a high protein concentration and the reconstituted formulation may be administered subcutaneously to the mammal to be treated herein.

Crystallized forms of the antibody or antibody are also contemplated. See, for example, U.S. 2002/0136719A1 (Shenoy et al.).

The formulation herein may also contain more than one active compound as necessary for the particular indication being treated, preferably those with complementary activities that do not adversely affect each other. For example, it may be desirable to further provide an anti-inflammatory agent, an anti-diabetic agent, and/or cholesterol-lowering drug of the “statin” class in the formulation. In some embodiments, the formulation comprises a second active agent, wherein the second active agent is insulin. In some embodiments, the insulin is rapid acting, short acting, regular acting, intermediate acting, or long acting insulin. In some embodiments, the insulin is and/or comprises Humalog, Lispro, Novolog, Apidra, Humulin, Aspart, regular insulin, NPH, Lente, Ultralente, Lantus, Glargine, Levemir, or Detemir. In some embodiments, the formulation comprises a second active agent, wherein the second active agent is a statin. In some embodiments, the statin is and/or comprises Atorvastatin (e.g., Lipitor or Torvast), Cerivastatin (e.g., Lipobay or Baycol), Fluvastatin (e.g., Lescol or Lescol), Lovastatin (e.g., Mevacor, Altocor, or Altoprev) Mevastatin, Pitavastatin (e.g., Livalo or Pitava), Pravastatin (e.g., Pravachol, Selektine, or Lipostat) Rosuvastatin (e.g., Crestor), Simvastatin (e.g., Zocor or Lipex), Vytorin, Advicor, Besylate Caduet or Simcor. The type and effective amounts of such other agents depend, for example, on the amount of antibody present in the formulation and clinical parameters of the subjects. These are generally used in the same dosages and with administration routes as used hereinbefore or about from 1 to 99% of the heretofore employed dosages. In some embodiments, the second active agent and the anti-oxidized LDL antibody are in a single pharmaceutical formulation. In some embodiments, the second active agent and the anti-oxidized LDL antibody are in separate formulations.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The formulations to be used for in vivo administration may be sterile. This is readily accomplished by filtration through sterile filtration membranes.

### V. Articles of Manufacture

The anti-oxidized LDL antibodies described herein may be contained within an article of manufacture comprising instructions for the methods and uses described herein. Preferably, the article of manufacture comprises: (a) a container comprising a composition comprising an anti-oxidized LDL antibody described herein and a pharmaceutically acceptable carrier or diluent within the container; and (b) a package insert with instructions for administering the composition to a subject suffering from insulin resistance and/or needing increased insulin sensitivity.

In some embodiments, the subject has metabolic syndrome. In some embodiments, the subject is at risk for developing metabolic syndrome. In some embodiments, the subject has one or more characteristics selected from the group consisting of (a) waist circumference of about 102 cm or more in men and about 88 cm or more in women, (b) fasting triglycerides of about 150 mg/dL or more, (c) a fasting glucose of about 95 mg/dL or higher, and (d) high levels of oxidized LDL. In some embodiments, the subject further has inflammation associated with diabetes. In some embodiments, the subject has a blood glucose level of about 95 mg/dL or higher after an overnight fast. In some embodiments, the subject has a blood glucose level of about 126 mg/dL or higher after an overnight fast. In some embodiments, the subject has a blood glucose level of about 140 mg/dL after a two-hour oral glucose tolerance test. In some embodiments, the subject has a blood glucose level of about 200 mg/dL after a two-hour oral glucose tolerance test. In some embodiments, the subject has pre-diabetes. In some embodiments, the subject has diabetes. In some embodiments, the diabetes is selected from the group consisting of type-I diabetes, type-II diabetes, and gestational diabetes. In some embodiments, the diabetes is type-II diabetes.

The article of manufacture comprises a container and a label or package insert on or associated with the container. Suitable containers include, for example, bottles, vials, syringes, etc. The containers may be formed from a variety of materials such as glass or plastic. The container holds or contains a composition that is effective for treating the insulin sensitivity and may have a sterile access port (for example the container may be an intravenous solution bag or a vial having a stopper pierceable by a hypodermic injection needle). At least one active agent in the composition is the antibody. The label or package insert indicates that the composition is used for treating insulin sensitivity in a subject suffering therefrom with specific guidance regarding dosing amounts and intervals of antibody and any other drug being provided. The article of manufacture may further comprise a second container comprising a pharmaceutically acceptable diluent buffer, such as bacteriostatic water for injection (BWFI), phosphate-buffered saline, Ringer's solution and dextrose solution. The article of manufacture may further include other materials desirable from a commercial and user standpoint, including other buffers, diluents, filters, needles, and syringes.

Optionally, the article of manufacture herein further comprises a container comprising another (e.g., second) agent other than the antibody for treatment and further comprising instructions on treating the mammal with such agent. In some embodiments, the second agent is an anti-inflammatory agent, an anti-diabetic agent, and/or cholesterol-lowering drug of the “statin” class. In some embodiments, the second active agent is insulin. In some embodiments, the insulin is rapid acting, short acting, regular acting, intermediate acting, or long acting insulin. In some embodiments, the insulin is and/or comprises Humalog, Lispro, Novolog, Apidra, Humulin, Aspart, regular insulin, NPH, Lente, Ultralente, Lantus, Glargine, Levemir, or Detemir. In some embodiments, the second active agent is a statin. In some embodiments, the statin is and/or comprises Atorvastatin (e.g., Lipitor or Torvast), Cerivastatin (e.g., Lipobay or Baycol), Fluvastatin (e.g., Lescol or Lescol), Lovastatin (e.g., Mevacor, Altocor, or Altoprev) Mevastatin, Pitavastatin (e.g., Livalo or Pitava), Pravastatin (e.g., Pravachol, Selektine, or Lipostat) Rosuvastatin (e.g., Crestor), Simvastatin (e.g., Zocor or Lipex), Vytorin, Advicor, Besylate Caduet or Simcor.

A “package insert” is used to refer to instructions customarily included in commercial packages of therapeutic products, that contain information about the indications, usage, dosage, administration, contraindications, other therapeutic products to be combined with the packaged product, and/or warnings concerning the use of such therapeutic products, etc.

Further details of the invention are illustrated by the following non-limiting Examples. The disclosures of all references in the specification are expressly incorporated herein by reference.

### EXAMPLES

The examples, which are intended to be purely exemplary of the invention and should therefore not be considered to limit the invention in any way, also describe and detail aspects and embodiments of the invention discussed above. The foregoing examples and detailed description are offered by way of illustration and not by way of limitation.

### Example 1

### Effect of Anti-Oxidized LDL Antibody on Serum Biomarkers and Insulin Resistance in High Fat and Fructose-Fed Rhesus Monkeys

To investigate the effects of anti-oxidized LDL antibodies on serum markers of inflammation, metabolism, pro-coagulant activity, and insulin resistance, high fat and fructose-fed rhesus monkeys were treated with anti-oxidized LDL antibodies.

### Experimental Design

Baseline Period (Re-Start Fructose; First Three Months)

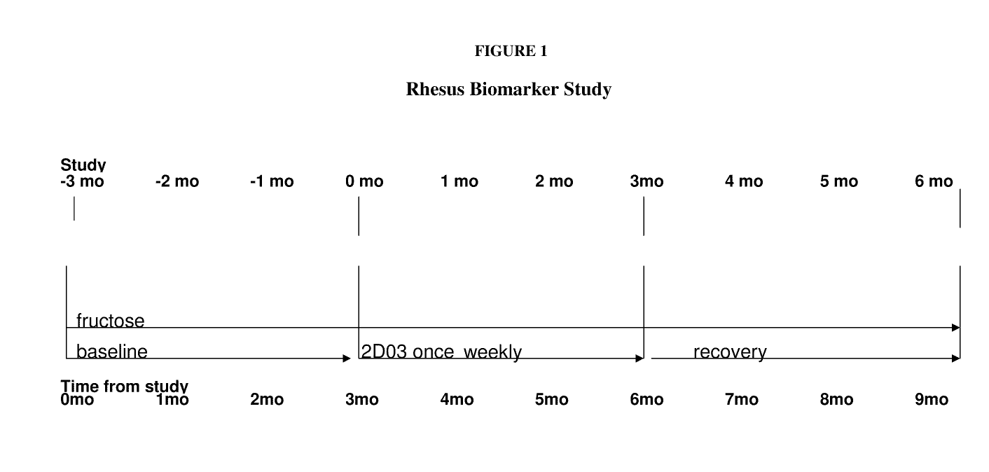

Prior to initiation of fructose supplementation, an intravenous glucose tolerance test (IVGTT), a dual-energy X-ray absorptiometry (DEXA) scan for body fat, and ultrasound measures of carotid intimal-medial thickness (CIMT) were performed once on each animal. At the same time (prior to glucose administration for IVGTT) urine samples were collected from each animal, by recovering 3-10 mL from the cage pan. Two blood samples (pre-fructose 1 and pre-fructose 2, described in Table 4 below; see also FIG. 1) were withdrawn from each of the 10 animals not less than 4 days apart. One may be at the time of sedation for IVGTT but prior to administration of glucose.

After scans were completed in all animals, and following the second blood sample, fructose supplementation to the seven high-fat and fructose-supplemented diet (“HFD”) animals were begun and continued through the end of the study (HFD=High fat+fructose diet, with limited exercise; animal numbers 19267, 20169, 20280, 20282, 20358, 21074, 2193). The three other animals were given a normal diet (Normal Diet group=normal diet+normal exercise; animal numbers 19836, 19843, 23527).

Blood samples were withdrawn from all 10 animals during the Baseline period as described in Table 4. See also FIG. 1. At the end of the three month Baseline period, all 10 animals again underwent IVGTT, DEXA, and CIMT, and a urine sample was collected.

Treatment Period

Upon completion of the baseline period, all 10 animals were treated with the anti-oxidized LDL antibody, 2D03, at a dosage of 10 mg/kg by IV, once every week for twelve weeks. The dose volume was adjusted weekly to body weight. The dose was administered intravenously via a superficial vein on the arm or leg (e.g. cephalic, saphenous) using disposable syringe and needle or butterfly catheter. Intravenous injection was followed by a saline flush of approximately 1 mL. The anti-oxidized LDL antibody formulation used in the experiment was 150 mg/ml of 2D03 in 20 mM Histidine Acetate, 150 mM Arginine Acetate, and 0.02% Polysorbate 20, pH 5.5.

A total of 13 doses were given to each animal. This first day of dosing was designated Study Day 0 Animals were dosed on Study Days 0, 7, 14, 21, 28, 35, 42, 49, 56, 63, 70, 77, and 84. Day of first dose will be Study Day 0. All days preceding Study Day 0 will be designated Study Day −1, Study Day −2, etc. All days following Study Day 0 will be designated Study Day 1, Study Day 2, etc. During the treatment period blood samples were withdrawn at various time points, as defined in Table 4 below. See also FIG. 1. In the week following administration of the final dose all animals again underwent IVGTT/DEXA/CIMT and urine sampling.

Post-Treatment Observation Period

Following administration of the final dose of the anti-oxidized LDL antibody, 2D03, all 10 animals were observed for twelve additional weeks. Blood samples were taken at various timepoints throughout the period, as described in Table 4 below. At the end of the observation period, a final IVGTT/DEXA/CIMT was completed, and a final urine sample was collected.

TABLE 4
Experimental Schedule

| | | | BLOOD |
|---|---|---|---|
| | | | VOL/ |
| SAMPLE TIME | STUDY | | SAMPLE |
| NAME | DAY | ASSAY | (ML) |
| Pre-fructose | | IVGTT/DEXA/CIMT/urine | 8 |
| pre-fructose 1 | | Elisa/RBM/chem | 19.5 |
| | | CBC/hgbA1c/LIPO | |
| | | Paxgene | |
| pre-fructose 2 | | Elisa | 16 |
| | | coag | |
| pre-fructose 3 | — | T-cell pheno/T-cell sorting | 9 |
| 12 weeks- START | −85 | | |
| FRUCTOSE | | | |
| 11 weeks | −77 | T-cell pheno/T-cell sorting | 9 |
| 10 weeks | −70 | Elisa | 16 |
| | | coag | |
| 9 weeks | −63 | | |
| 8 weeks | −56 | Elisa/RBM/chem | 17 |
| | | CBC/hgbA1c/LIPO | |
| 7 weeks | −49 | | |
| 6 weeks | −42 | Elisa | 16 |
| | | coag | |
| 5 weeks | −35 | T-cell pheno/T-cell sorting | 9 |
| 4 weeks | −28 | Elisa/RBM/chem | 17 |
| | | CBC/hgbA1c/LIPO | |
| 3 weeks | −21 | | |
| 2 weeks | −14 | Elisa/ | 16 |
| | | coag | |
| 1 week | −7 | T-cell pheno/T-cell sorting | 9 |
| Pre-dose | | IVGTT/DEXA/CIMT/urine | 8 |
| Week 0 | 0 | Elisa/RBM/chem | 20.5 |
| (Pre- | | CBC/hgbA1c/LIPO | |
| dose 1) | | Paxgene | |
| | | PK/ATA | |

Time = 0 FIRST DOSE ADMINISTRATION

| | | | |
|---|---|---|---|
| 5 mins post 1st dose | 0 | PK/ATA | 1 |
| 8 hours post 1st dose | 0 | PK/ATA | 1 |
| 24 hours post-dose | 1 | PK/ATA | 1 |
| (day 1) | | | |
| 72 hours post-dose | 3 | PK/ATA | 1 |
| (day 3) | | | |
| week 1 (pre-dose 2) | 7 | PK/ATA | 1 |
| week 2 (pre-dose 3) | 14 | PK/ATA | 17 |
| | | Elisas | |
| | | coag | |
| week 3 (pre-dose 4) | 21 | PK/ATA | 1 |
| week 4 (pre-dose 5) | 28 | Elisa/RBM/chem | 18 |
| | | CBC/hgbA1c/LIPO | |
| | | PK/ATA | |
| week 5 (pre-dose 6) | 35 | PK/ATA | 1 |
| week 6 (pre-dose 7) | 42 | PK/ATA | 26 |
| | | Elisas | |
| | | coag | |
| | | T-cell pheno/T-cell sorting | |
| week 7 (pre-dose 8) | 49 | PK/ATA | 1 |
| (week 8 pre-dose 9) | 56 | Elisa/RBM/chem | 18 |
| | | CBC/hgbA1c/LIPO | |
| | | PK/ATA | |
| week 9 (pre-dose 10) | 63 | PK/ATA | 1 |
| week 10 (pre-dose 11) | 70 | PK/ATA | 17 |
| | | Elisas | |
| | | coag | |
| week 11 (pre-dose 12) | 77 | PK/ATA | 11 |
| week 12 (pre-dose 13) | 84 | Elisa/RBM/chem | 18 |
| | | CBC/hgbA1c/LIPO | |
| | | PK/ATA | |
| 5 mins post last dose | 84 | PK/ATA | 1 |
| 8 hours post last dose | 84 | PK/ATA | 1 |
| 24 hours post last dose | 85 | PK/ATA | 1 |
| Post-dose | | IVGTT/DEXA/CIMT/urine | 8 |
| 3 days post last dose | 87 | PK/ATA | 12.5 |
| | | Paxgene | |
| | | T-cell pheno/T-cell sorting | |
| week 13 (1 week post | 91 | PK/ATA | 1 |
| last dose) | | | |
| week 14 | 98 | PK/ATA/ | 17 |
| 2 wks post-dosing) | | Elisas | |
| | | coag | |
| week 15 | 105 | | |
| week 16 | 112 | Elisa/RBM/chem | 18 |
| (4 wks post-dosing) | | CBC/hgbA1c/LIPO | |
| | | PK/ATA | |
| week 17 | 119 | | |
| week 18 | 126 | PK/ATA/ | 17 |
| (6 wk post-dosing) | | Elisas | |
| | | coag | |
| week 19 | 133 | | |
| week 20 | 140 | Elisa/RBM/chem | 18 |
| (8 wk post-dosing) | | CBC/hgbA1c/LIPO | |
| | | PK/ATA | |
| week 21 | 147 | | |
| week 22 | 154 | PK/ATA | 26 |
| (10 wk post-dosing) | | Elisas | |
| | | coag | |
| | | T-cell pheno/T-cell sorting | |
| week 23 | 161 | | |
| week 24 | 168 | Elisa/RBM/chem | 20.5 |
| | | CBC/hgbA1c/LIPO | |
| | | Paxgene | |
| | | PK/ATA | |
| End-of-study | | IVGTT/DEXA/CIMT/urine | 8 |

### Materials and Methods

Animals Used in Experiments

Macaca mulatta (rhesus monkey) were used in the experiments. The rhesus monkey is an accepted nonhuman primate model that responds to this class of pharmaceuticals. With high fat and fructose feeding, along with limitations on exercise, these animals have been shown in previous studies to exhibit most of the hallmarks of insulin resistance seen in humans with insulin resistance. The use of these monkeys will maximize the likelihood of identifying experimental responses that are quantitatively and qualitatively similar to those which may be expected to be seen in humans.

The body weight range of these monkeys at the time of dosing ranged from 14.4 to 21 kg for HFD animals and 7.8 to 9.8 for normal diet group. These monkeys were all male, non-naïve animals from 9.5 to 12.5 years of age.

HFD and Normal Diet

Animals in the HFD group were fed Customized Primate Research Center Diet 5A1F (Lab Diet, Inc) Animals were offered up to 20 biscuits per day, half in the morning and half in the afternoon. These animals were also fed peanut butter treat (300 kcal) and a half of an apple every day Animals were fed their morning meal at 10:00 hours, the remains of the morning meal is removed at 14:00 hours and weighed. The evening meal was fed at 15:00 hours and the remains of the meal removed at 17:00 hours. Animals did not have food available during the lights outs period. Each animal in the HFD group also received 500 mL of 10% fructose in cherry Kool-Aid three times per week.

Animals in the normal diet group were fed Monkey Diet #5038 or Monkey Diet Jumbo #5037 (Lab Diet Inc.). Animals were offered up to 20 biscuits per day, half in the morning and half in the afternoon. Animals in the normal diet group did not receive fructose supplementation. They were fed in the same manner as above.

Food intake for each animal (biscuit count) was monitored and recorded (with the exception of weekends) throughout the study.

To provide fasting blood samples, food was withheld from animals overnight (following the evening feeding) until after the blood collection on days of sampling.

Glucose and Insulin Tolerance Tests

IVGTTs were performed by measuring blood glucose clearance after an IV bolus infusion of sterile 50% dextrose solution (600 mg/kg). Animals were fasted overnight before the test and sedated with Telezol (3 mg/kg) for the procedure. A 1 mL blood sample was collected before administration of the dextrose and at 1, 3, 5, 10, 20, 40, and 60 minutes after administration of dextrose. Blood glucose was measured immediately in whole blood with a glucometer (Onetouch Ultra Blood Glucose Monitor; LifeScan; Milpitas, Calif.). Plasma was assayed for insulin by the ONPRC/OHSU Endocrine Services Laboratory (Portland, Oreg.) using an Immulite 2000. The insulin and glucose area under the curve (AUC), where glucose and insulin levels are plotted against time, were calculated using Prism GraphPad software.

DEXA Scans

For body composition analysis, animals were sedated with Telezol (3 mg/kg) and the sedation maintained with ketamine. The animals were laid flat on the DEXA (Hologic Discovery A) and a whole body scan was performed. Central fat mass were calculated as well as whole body fat. This was compared to central and total lean mass.

Carotid Ultrasound Scans

The animals were sedated with Ketamine (10 mg/kg). The carotid arteries were imaged with a Siemens Doppler Ultrasound machine. The carotid intimal thickness were calculated by the automated software package from Siemens.

Body Weight

During the treatment period, all animals were weighed before feeding on the day before dosing in order to adjust test article volume to maintain the 10 mg/kg dose. During baseline and post-treatment observation periods, animals were weighed once every two weeks.

Urine Sampling

Urine samples were collected from each animal pre-fructose, week −1, week 12 (post-last-dose), and week 24. Three −10 mL clean urine was collected from each animal by recovery from the cage pan. Urine was centrifuged to remove large debris, then placed in a clean tube labeled with the animal number, study day, date, and sample type (“urine”), and stored at −70° C.

Blood Sampling and Processing

Venous blood samples were withdrawn from each of the 10 animals on study as shown in Table 4.

Blood collected in RTT (serum) was allowed to clot at room temperature for 20-30 minutes, then centrifuged to yield serum. The serum was decanted into clean plastic tubes in approximately 1 mL aliquots. Tubes were clearly labeled with animal number, sample timepoint, sample description (“serum”), and date. The serum was stored at approximately 70° C. until shipped on dry ice. These samples were assayed for various biomarkers, test article and anti-test article antibody levels, and serum chemistry profile.

Blood collected in LTT (EDTA) for Complete Blood Cell Count and Hemoglobin A1c was refrigerated and transferred for assay.

Blood collected in LTT (EDTA) for LipoScience's Lipid Profile Analysis was centrifuged to yield plasma. The plasma was decanted into clean plastic tubes labeled with animal number, sample timepoint, sample description (“EDTA plasma”), and date. Tubes were stored at 4° C. (refrigerated) and shipped overnight on wet ice for analysis.

Blood collected in PaxGene tubes was allowed to sit at room temperature for 2 hours after collection to allow for red blood cell lysis. Tubes were then transferred to the 70° C. freezer for future microarray analysis.

Blood collected in GTT (lithium heparin) was transferred to the immunology specialist for T-cell assays. Cells recovered from the T-cell sorting were stored at 70° C. for future cytokine expression/release analysis.

Blood collected in BTT (sodium citrate) was centrifuged to yield plasma. The plasma was decanted into clean plastic tubes labeled with animal number, sample timepoint, sample description (“citrate plasma”), and date, then stored at 70° C.

Assays

Serum 2D03 Concentration

Serum samples for measurement of 2D03 concentration were stored at 70° C. and defrosted just before assay. The concentration of 2D03 in each serum sample was determined using an antigen binding based ELISA method. The assay used MDA ApoB100 (Academy BioMedical Company, Inc.; Houston, Tex.) as the capture reagent and a rabbit anti-human IgG HRP (Dako; Glostrup, Denmark) as the detection reagent. The reportable range of the assay was 1.4 to 195 ng/mL in well. A minimum serum sample dilution of 1:200 was used for all samples. The minimum quantifiable concentration was 280 ng/mL.

Anti-Therapeutic Antibody Assay

Anti-therapeutic antibody (ATA) levels were measured from serum stored at 70° C. and defrosted prior to testing using an assay developed for the Meso Scale Discovery (MSD) electrochemiluminescence platform (Gaithersburg, Md.).

Rules Based Medicine Multi Analyte Profile

Frozen serum was submitted to Rules Based Medicine Inc. (Austin, Tex.) for the Human Multi Analyte Profile (version 1.6), a proprietary Luminex based assay of 89 different biomarkers. Several pivotal analytes were also measured by an ELISA using specific rhesus antibodies or human antibodies proven to capture the rhesus analyte.

Enzyme Linked Immunoabsorbent Assays

All assays were performed using commercially available kits and according to the manufacturer's directions. Assays either used rhesus specific antibodies or human antibodies proven to capture the rhesus analyte. Some analytes were identical to those also measured in the Rules Based Medicine multi analyte panel. The following analytes were measured by ELISA in serum which had been stored at 70° C. and defrosted prior to assay:
Oxidized LDL: Oxidized LDL Competitive ELISA kit, Mercodia, Inc., Catalog No. 10 1158 01
Granulocyte Macrophage Colony Stimulating Factor (GM-CSF): Monkey GM CSF kit, Cell Sciences, Inc., Catalog No. CKM000
Interferon gamma IFN-γ: Interferon gamma (Monkey) EIA kit, ALPCO Immuno assays, Inc., Catalog No. 45 IFNMK E01
Tumor Necrosis Factor alpha (TNF-α): TNF-α Monkey ELISA Kit, Invitrogen Inc., Catalog No. KPC3011
Adiponectin: Human Serum Adiponectin Kit Acrp30, Alpha Diagnostic International, Inc. Catalog No. 100 140 ADH
C Reactive Protein (CRP): Monkey C Reactive Protein Kit, Alpha Diagnostics Inc., Catalog No. 1050
Interleukin-18 (IL-18): IL-18 Kit, MBL, Inc., Catalog No. 7620
Interleukin-8 (IL-8): Interleukin-8 ELISA Kit, R&D, Inc., Catalog No. D8050
Soluble CD40 (sCD40): Soluble CD40 ELISA kit, Kamya Biomedical, Catalog No. KT 004
Interleukin-1 receptor antagonist (IL-1ra): IL 1ra kit, Biosource International (now Invitrogen), Catalog No. KAC1181
Monocyte chemoattractant protein 1 (MCP-1): Monocyte chemoattractant protein 1 ELISA kit BD OptEIA, Becton Dickinson, Catalog No. 559017
Interleukin 6 (IL-6): Monkey Interleukin 6 ELISA Kit, Cell Sciences, Inc., Catalog No. CKM005
Interleukin 1 beta (IL-1β): Monkey Interleukin 1 beta ELISA Kit, Cell Sciences, Inc., Catalog No. CKM039

Lipoprotein Particle Analysis

Fresh, EDTA anticoagulated plasma (refrigerated) was shipped overnight to LipoScience for nuclear magnetic resonance (NMR) analysis of lipoprotein subfractions.

Concentrations of the following 10 subclass categories were measured: large VLDL (including chylomicrons, if present) (>60 nm), medium VLDL (35-60 nm), small VLDL (27-35 nm), IDL (23-27 nm), large LDL (21.2-23 nm), medium small LDL (19.8-21.2 nm), very small LDL (18-19.8 nm), large HDL (8.8-13 nm), medium HDL (8.2-8.8 nm), and small HDL (7.3-8.2 nm). Since levels of the “medium small” and “very small” LDL subclasses have been found to have very similar correlations with lipid levels and coronary disease and diabetes outcomes, they were grouped into a single “small LDL” subclass (18-21.2 nm). VLDL and LDL subclass particle concentrations are given in units of nmol/L and those of HDL subclasses in μmol/L. Further summation of the subclass levels also provided total VLDL, LDL (including IDL), and HDL particle concentrations. Weighted average VLDL, LDL, and HDL particle sizes (in nm diameter units) were computed as the sum of the diameter of each subclass multiplied by its relative mass percentage as estimated from the amplitude of its NMR signal.

Serum Chemistry Panel

A standard serum chemistry profile of 17 analytes plus HDL, LDL, cholesterol, and triglycerides was performed using the Roche Cobas Integra 400 multi channel chemistry analyzer (Roche, Inc.; Indianapolis, Ind.) from serum that had been stored at 70° C. and defrosted just prior to assay. Insulin levels were measured from the same defrosted serum using a Siemens Immulite 2000 immunoassay analyzer (Siemens, Inc.; Piscataway, N.J.).

Urinalysis

Urine which had been stored at 70° C. was defrosted and tested by dipstick (Multistix SG 10, Siemens, Inc.; Piscataway N.J.).

Complete Blood Cell Count

A standard panel of 10 blood parameters was performed on fresh, EDTA anticoagulated whole blood using a Horiba ABX Pentra 60 C (Horiba ABX; Irvine, Calif.).

Hemoglobin A1c

HbA1c levels were measured from EDTA anticoagulated whole blood using an A1cNow kit from Bayer (Pittsburgh, Pa.) and following manufacturers' directions.

T-Cell Phenotyping and Sorting

T-cell phenotyping was performed before the start of fructose feeding, before the start of dosing, during dosing Week 6 (before administration of dose 7), and 10 weeks following the final dose. Peripheral blood mononuclear cells (PBMCs) were isolated from whole blood after centrifugation over Ficoll-Histopaque (Sigma; St Louis, Mo.). Cells were frozen for subsequent analysis by using a controlled cyropreservation apparatus procedure (CryoMed Freezer No. 7454; ThermoForma; Marietta, Ohio). All antibodies were purchased from PharMingen (San Diego, Calif.), Ebioscience (San Diego, Calif.), or Caltag (Burlingame, Calif.) and were used in accordance with manufacturers' recommendations. Samples were collected using FACSCalibur or FACSLSRII (Becton Dickinson; San Jose, Calif.), and data were analyzed using CellQuest (BD Biosciences; Mountain View, Calif.) or FlowJo (Treestar; Ashland, Oreg.), with a minimum of 10⁵ events collected per sample.

Pharmacokinetic Analysis

The pharmacokinetic (PK) parameters of 2D03 were calculated by non compartmental analysis using WinNonlin Professional Edition (version 5.2.1, Pharsight Corporation; Mountain View, Calif.). Nominal doses and sampling times were used.

The following parameters were estimated:
Cₘₐₓ—Maximum observed concentration in serum
t_{1/2}—Elimination half life
AUC₀₋₇—Area under the serum concentration time curve from Days 0 to 7, estimated by the linear trapezoidal rule
AUC_{τ}—Area under the serum concentration-time curve from Days 84 to 91 signifying AUC at steady state, estimated by the linear trapezoidal rule.
CLₛₛ—Clearance at steady state
Vₛₛ—Volume of distribution at steady state
AR—Accumulation ratio for C_{trough} (ratio of predose concentrations at Days 84 and 7).

Statistical Analysis

Occasional estimates of significance were calculated using a paired t-test evaluation without applying a correction for multiple measures.

### Results

IVGTT

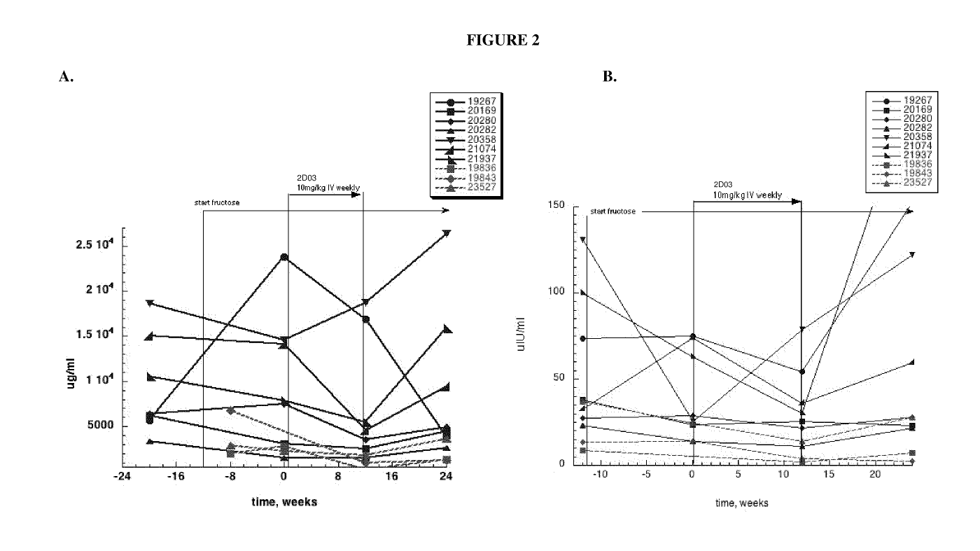

Results of the glucose tolerance tests are shown in Table 5. There was improvement in insulin sensitivity (reduction in insulin AUC) in 8 of 10 animals during the 2D03 treatment period, with all but 1 animal showing a return toward baseline (increase in) insulin AUC after the 12 week washout (FIG. 2A). Pre-IVGTT serum glucose levels did not change during this study. Four of 7 HFD animals had elevated or very variable pre-IVGTT plasma insulin levels throughout the study. The other 3 HFD animals had plasma insulin levels similar to the 3 control monkeys. Pre-IVGTT insulin decreased in 7 of 10 animals during treatment with 2D03, and then increased in 6 of 10 animals following washout (FIG. 2B).

Insulin sensitivity as indicated by IVGTT was increased after treatment with 2D03 in the high fructose diet animals with or without elevated insulin levels (FIG. 3A). Further, glucose tolerance as measured by insulin AUC using IVGTT experiments was reduced after treatment with 2D03 in both the high fructose diet and normal diet animals (FIG. 3B).

TABLE 5

| animal | | June | | | November | | | January | | | April | |
|---|---|---|---|---|---|---|---|---|---|---|---|---|
| number | | 2008 | | | 2008 | | | 2009 | | | 2009 | |
| 19267 | | BASELINE | AUC | | BASELINE | AUC | | BASELINE | AUC | | BASELINE | AUC |
| | GLU- | 45 | 4908 | GLU- | 49 | 5119 | GLU- | 45 | 4996 | GLU- | 61 | 4884 |
| | COSE | | | COSE | | | COSE | | | COSE | | |
| | INSU- | 73.5 | 5701 | INSU- | 75.1 | 23796 | INSU- | 54.2 | 16919 | INSU- | 152 | 3795 |
| | LIN | | | LIN | | | LIN | | | LIN | | |
| | | September | | | November | | | January | | | April | |
| | | 2008 | | | 2008 | | | 2009 | | | 2009 | |
| 19836 | | BASELINE | AUC | | BASELINE | AUC | | BASELINE | AUC | | BASELINE | AUC |
| | GLU- | 59 | 5081 | GLU- | 47 | 3550 | GLU- | 53 | 2870 | GLU- | 54 | 4353 |
| | COSE | | | COSE | | | COSE | | | COSE | | |
| | INSU- | 8.8 | 1977 | INSU- | 147 | 2769 | INSU- | 2 | 200.5 | INSU- | 7.1 | 1351 |
| | LIN | | | LIN | | | LIN | | | LIN | | |
| | | | | Value error. Most likely 14.7, | | | | | | | | |
| | | | | GNE sample has low insulin | | | | | | | | |
| | | September | | | November | | | January | | | April | |
| | | 2008 | | | 2008 | | | 2009 | | | 2009 | |
| 19843 | | BASELINE | AUC | | BASELINE | AUC | | BASELINE | AUC | | BASELINE | AUC |
| | GLU- | 60 | 6753 | GLU- | 48 | 4662 | GLU- | 53 | 3817 | GLU- | 45 | 5727 |
| | COSE | | | COSE | | | COSE | | | COSE | | |
| | INSU- | 13.4 | 1956 | INSU- | 14.2 | 1352 | INSU- | 4 | 928.4 | INSU- | 2.59 | 1229 |
| | LIN | | | LIN | | | LIN | | | LIN | | |
| | | June | | | November | | | January | | | April | |
| | | 2008 | | | 2008 | | | 2009 | | | 2009 | |
| 20169 | | BASELINE | AUC | | BASELINE | AUC | | BASELINE | AUC | | BASELINE | AUC |
| | GLU- | 53 | 5438 | GLU- | 55 | 6147 | GLU- | 66 | 5919 | GLU- | 54 | 6459 |
| | COSE | | | COSE | | | COSE | | | COSE | | |
| | INSU- | 38.1 | 6204 | INSU- | 23.6 | 3119 | INSU- | 25.4 | 2498 | INSU- | 23 | 4423 |
| | LIN | | | LIN | | | LIN | | | LIN | | |
| | | June | | | November | | | January | | | April | |
| | | 2008 | | | 2008 | | | 2009 | | | 2009 | |
| 20280 | | BASELINE | AUC | | BASELINE | AUC | | BASELINE | AUC | | BASELINE | AUC |
| | GLU- | 47 | 6250 | GLU- | 55 | 6347 | GLU- | 62 | 4634 | GLU- | 50 | 6485 |
| | COSE | | | COSE | | | COSE | | | COSE | | |
| | INSU- | 27.3 | 6371 | INSU- | 28.7 | 7484 | INSU- | 21.5 | 3566 | INSU- | 27.7 | 4916 |
| | LIN | | | LIN | | | LIN | | | LIN | | |
| | | June | | | November | | | January | | | April | |
| | | 2008 | | | 2008 | | | 2009 | | | 2009 | |
| 20282 | | BASELINE | AUC | | BASELINE | AUC | | BASELINE | AUC | | BASELINE | AUC |
| | GLU- | 61 | 5141 | GLU- | 62 | 4948 | GLU- | 63 | 3557 | GLU- | 68 | 7755 |
| | COSE | | | COSE | | | COSE | | | COSE | | |
| | INSU- | 23.1 | 3401 | INSU- | 14 | 1541 | INSU- | 11 | 1560 | INSU- | 21.6 | 2608 |
| | LIN | | | LIN | | | LIN | | | LIN | | |
| | | June | | | November | | | January | | | April | |
| | | 2008 | | | 2008 | | | 2009 | | | 2009 | |
| 20358 | | BASELINE | AUC | | BASELINE | AUC | | BASELINE | AUC | | BASELINE | AUC |
| | GLU- | 41 | 4464 | GLU- | 57 | 5339 | GLU- | 95 | 5030 | GLU- | 57 | 3940 |
| | COSE | | | COSE | | | COSE | | | COSE | | |
| | INSU- | 131 | 18626 | INSU- | 25.6 | 14628 | INSU- | 78.8 | 18705 | INSU- | 122 | 26390 |
| | LIN | | | LIN | | | LIN | | | LIN | | |
| | | June | | | November | | | January | | | April | |
| | | 2008 | | | 2008 | | | 2009 | | | 2009 | |
| 21074 | | BASELINE | AUC | | BASELINE | AUC | | BASELINE | AUC | | BASELINE | AUC |
| | GLU- | 48 | 6653 | GLU- | 57 | 4510 | GLU- | 50 | 6001 | GLU- | 46 | 6248 |
| | COSE | | | COSE | | | COSE | | | COSE | | |
| | INSU- | 32.9 | 15077 | INSU- | 74.2 | 14150 | INSU- | 36.3 | 4554 | INSU- | 49.7 | 9471 |
| | LIN | | | LIN | | | LIN | | | LIN | | |
| | | June | | | November | | | January | | | April | |
| | | 2008 | | | 2008 | | | 2009 | | | 2009 | |
| 21937 | | BASELINE | AUC | | BASELINE | AUC | | BASELINE | AUC | | BASELINE | AUC |
| | GLU- | 44 | 5229 | GLU- | 51 | 5773 | GLU- | 57 | 4568 | GLU- | 48 | 5122 |
| | COSE | | | COSE | | | COSE | | | COSE | | |
| | INSU- | 100 | 10561 | INSU- | 63.2 | 5737 | INSU- | 30.3 | 5479 | INSU- | 216 | 15886 |
| | LIN | | | LIN | | | LIN | | | LIN | | |
| | | September | | | November | | | January | | | April | |
| | | 2008 | | | 2008 | | | 2009 | | | 2009 | |
| 23527 | | BASELINE | AUC | | BASELINE | AUC | | BASELINE | AUC | | BASELINE | AUC |
| | GLU- | 60 | 6197 | GLU- | 57 | 3810 | GLU- | 53 | 2994 | GLU- | 60 | 2948 |
| | COSE | | | COSE | | | COSE | | | COSE | | |
| | INSU- | 37.1 | 2880 | INSU- | 24.6 | 2268 | INSU- | 13.8 | 1837 | INSU- | 27.9 | 3590 |
| | LIN | | | LIN | | | LIN | | | LIN | | |

AUC = area under the curve;
baseline = pre-test plasma concentration (mg/dL or uIU/mL);
IVGTT = intravenous glucose tolerance test.
Note:
Values are in day · μg/mL.

DEXA

There was no marked change in either percent total body fat or percent trunk fat attributable to 2D03 treatment. Two control animals (Animals 19836 and 23527) increased their body fat markedly during the 9 month study.

Body Weight

Average weight change for the HFD group was +0.5 kg (range −0.2 to +0.8) during the experimental period from −9 weeks to 24 weeks. While 1 control animal (Animal 19843) showed no weight change over that time period, Animal 19836 gained 1.6 kg and Animal 23527 gained 2.7 kg. In spite of continuing consumption of the high fat diet, the HFD animals did not gain as much weight as 2 of 3 control animals.

Food Intake

Food intake did not change during the experiment.

Assays

Serum 2D03 Concentration

Mean 2D03 Cₘₐₓ following the first IV dose was 236±30 μg/mL. Mean 2D03 Cₘₐₓ following the final IV dose was 286±46 μg/mL. All animals achieved trough serum levels of 2D03 between approximately 36 and 49 μg/mL following the first dose and between 23 and 137 μg/mL following the penultimate dose.

Anti Therapeutic Antibody (ATA) Assay

Antibodies to 2D03 were detected in the serum from Animal 19267 on Days 112, 126, and 140, which led to a decline in serum concentrations in that monkey beyond Day 14. The serum concentrations for this animal were constantly lower than those for the rest of the animals Additionally, Animals 20169 and 21937 in the HFD group and Animals 19836 and 19843 in the control group were also positive for antibodies to 2D03 during the recovery phase; however, the serum 2D03 concentrations were similar to those monkeys who did not test positive for ATAs.

Rules Based Medicine Multi Analyte Assay

89 parameters were tested by Rules Based Medicine. The following analytes showed a marked change during the treatment period: GM-CSF, EN-RAGE, FGF-basic, IL-1β, TNF-α, IL-13, IL-15, interferon-γ, and thrombopoietin.

GM-CSF levels spiked in all animals at the 8 week timepoint, were returning toward baseline at the end of the treatment period, and had returned to baseline value by 4 weeks.

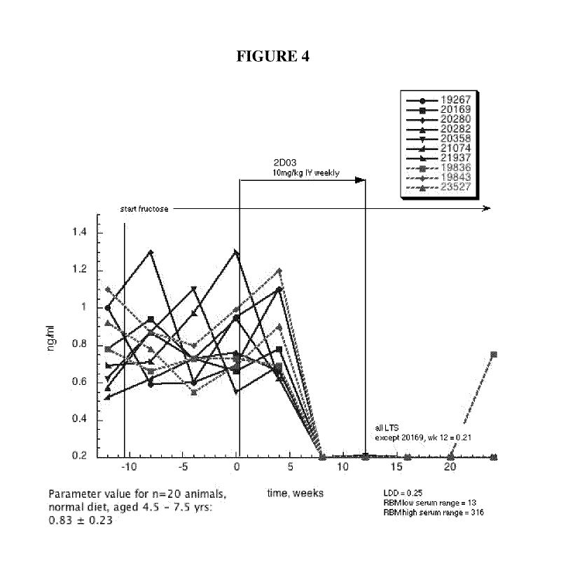

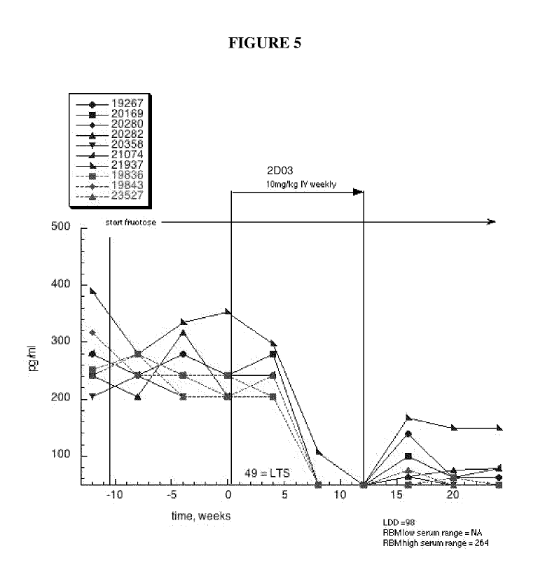

EN-RAGE, FGF-basic, IL-1β, and TNF-α levels all decreased during 2D03 treatment and remained low through the end of the washout period, although FGF-basic showed some return toward baseline values (see e.g., FIGS. 4 and 5).

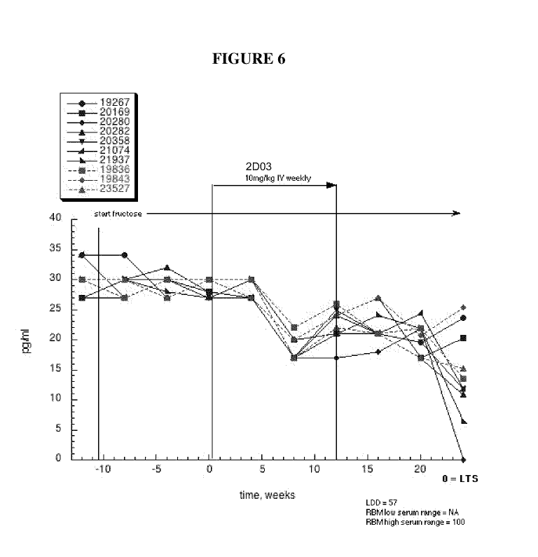

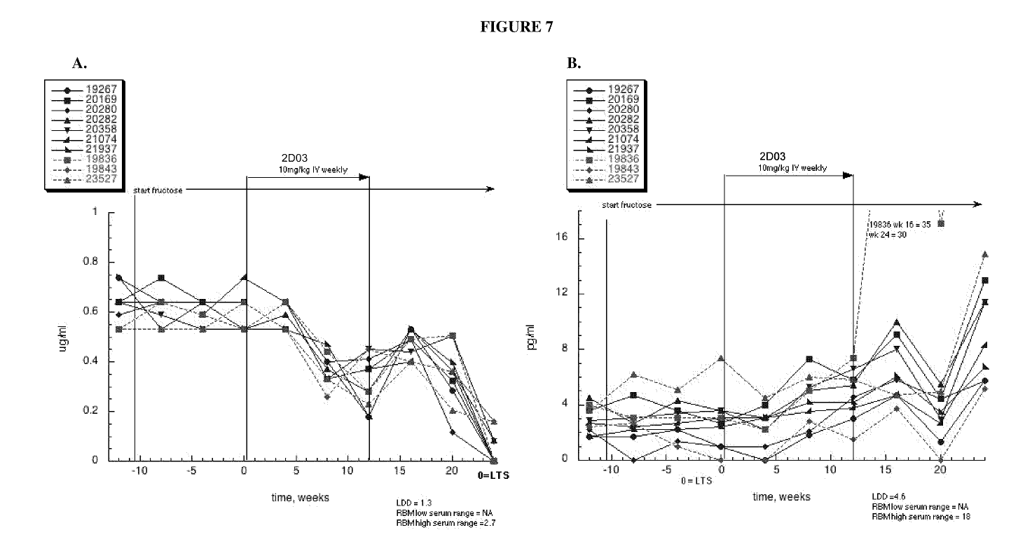

Values for IL-13 and IL-15 were close to the limits of detection in the RBM assay but both showed a decrease during 2D03 treatment, which persisted through the washout period (FIGS. 6 and 7A, respectively). Interferon-γ levels rose slightly during the treatment period and continued to rise for the first four weeks of the washout period, then showed a brief decline at Week 20, and another increase at Week 24 (FIG. 7B).

Thrombopoietin levels increased during 2D03 treatment and returned to baseline during the washout period.

Enzyme Linked Immunoabsorbent Assays

Oxidized LDL as measured by an ELISA was variable over the experimental period but no clear effect on this analyte was seen during 2D03 treatment.

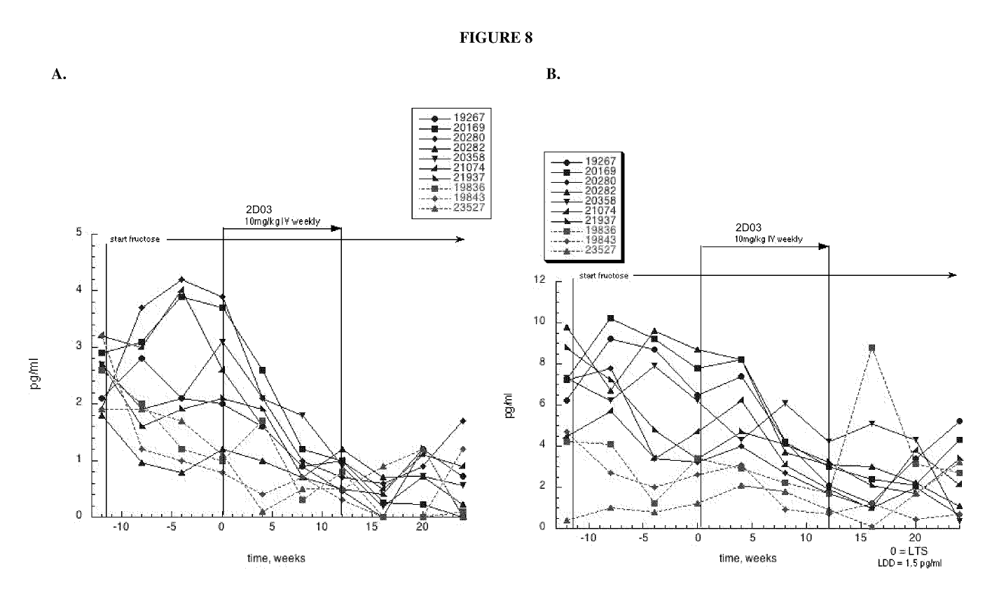

Following the start of fructose supplementation, HFD animals displayed higher IL-1β levels than control animals. These levels decreased markedly, to the levels of the control animals, during the 2D03 treatment period. This reduction was maintained through the end of the washout period (FIG. 8A). A marked decrease in IL-1β was also measured in the RBM assay (see FIG. 5B); however, the separation in pre-treatment values between HFD and control groups was not evident in those results. Absolute values measured were also somewhat lower.

Pre treatment levels of TNF-α were elevated in HFD animals compared with control. These levels were reduced markedly during the treatment period, and the reduction was maintained through the washout period (FIG. 8B).

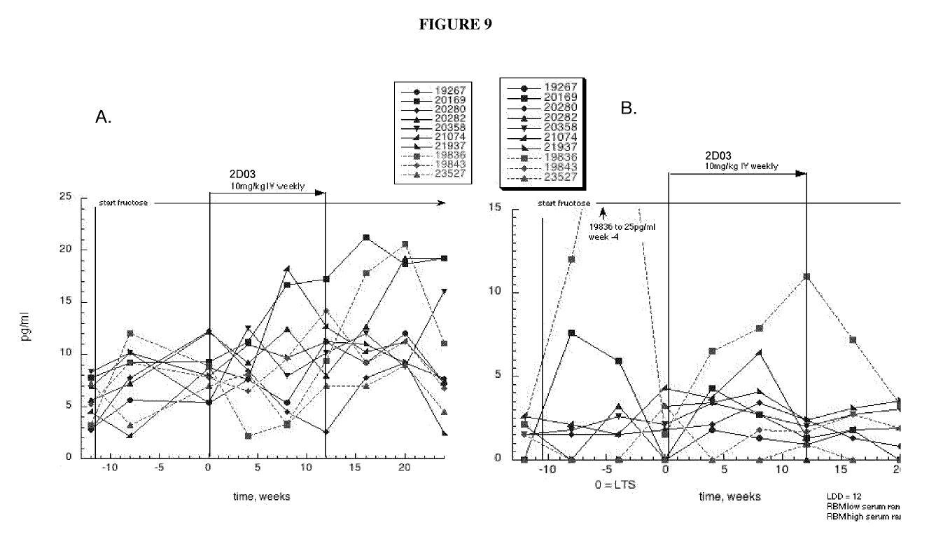

IL-6, as measured by an ELISA, showed a small increase in variability during the 2D03 treatment period, and persisted through the washout period. IL-6 levels measured in the RBM panel were highly variable but did not increase in variability during dosing (FIG. 9A-B).

There was no change in IL-1 receptor antagonist (IL-1ra) level during the study. here was no marked change in adiponectin, CRP, IL-18, or soluble CD40 ligand in any animal during the 9 months of this study. This is in agreement with the results for these analytes obtained in the Rules Based Medicine multi analyte profile.

Lipoprotein Particle Analysis

There was no marked change in any measured lipid subfraction during the period of treatment with 2D03. In HFD animals, small VLDL particle concentrations and total VLDL particles and chylomicrons trended lower beginning shortly after the start of fructose supplementation, continued to decrease slightly throughout the treatment period, and appeared to level off during the washout period.

At the pre-fructose timepoint, IDL particles in 5 of 7 HFD animals were markedly elevated compared with the control animals In all but 1 of these animals, the IDL level began to drop at the first timepoint following addition of fructose to the diet. In the remaining animal, the IDL dropped by the second timepoint following fructose addition. IDL levels of all monkeys then continued to lower for the remainder of the study. Treatment with 2D03 did not appear to affect this parameter.

Serum Chemistry Panel

BUN was low in HFD animals compared to normal diet animals throughout the study (HFD 7.5±1.7 mg/dL versus control 16.9±2.7 mg/dL pre-treatment). In normal diet animals, although never abnormally high, BUN increased slightly during the treatment period (HFD max 11.3±3.3 mg/dL (Week 12) versus control max 24±6.5 mg/dL (Week 12)), but returned to baseline within 4 weeks post-final dose. Serum creatinine was never outside normal values but increased slightly in most animals during the treatment period (HFD 0.93±0.09 mg/dL pre-treatment to max 1.19±0.06 mg/dL (Week 12) versus control 0.85±0.1 mg/dL pre-treatment to max 1.14±0.16 mg/dL (Week 12)). This parameter returned to baseline by 4 weeks post-treatment.

The urine protein:creatinine ratio was not markedly altered during the treatment period. Serum albumin decreased slightly in all animals following 4 weeks of 2D03 treatment, then increased to a maximum of approximately 125% of pre-treatment baseline 4 weeks following the final dose, returning to baseline value by the end of the washout period. Serum calcium levels showed a drop from the pre-treatment mean of 5.9±0.25 mg/dL (HFD) and 5.7±0.72 mg/dL (control) to 4.4±0.8 mg/dL (HFD) and 5.0±0.4 mg/dL (control) at Week 8, followed by an increase to 8.5±0.7 mg/dL (HFD) and 8.0±0.2 mg/dL (control) at Week 12. The increase persisted to the end of the study. Serum inorganic phosphorus did not change during the experiment. Baseline serum chloride was 96.7±2.3 mg/dL (HFD) and 94.1±5.6 mg/dL (control). Serum chloride increased slowly through the treatment period and this persisted through the washout phase (max 110.3±2.6 mg/dL HFD (Week 24); max 110.3±3.2 mg/dL control (Week 24)). There was an increase in variability of sodium levels over the same time period.

Urinalysis

Most HFD and control animals showed an increase in urine protein content between the pre-fructose and pre-treatment phase timepoints. All but 1 animal displayed a drop in urine protein during 2D03 treatment (from the predose to the Week 12 timepoints). The protein levels in most animals then increased toward predose levels by the end of the 12 week washout.

Two of the HFD animals were positive for urine glucose at the pre-fructose timepoint. No glucose was detected in any urine sample either at the pre-dose or post-treatment period timepoints. By the end of the washout period, however, 4 HFD animals had positive urine glucose results.

Complete Blood Cell Count

There was no marked change in any measured parameter throughout the study period. Although thrombopoietin levels as measured in the RBM assay rose during the treatment phase, platelet counts were stable over the 9 months of the study.

Hemoglobin A1c

Hemoglobin A1c was not elevated in any animal at the start of the study and did not change upon treatment.

T-Cell Phenotyping and Sorting

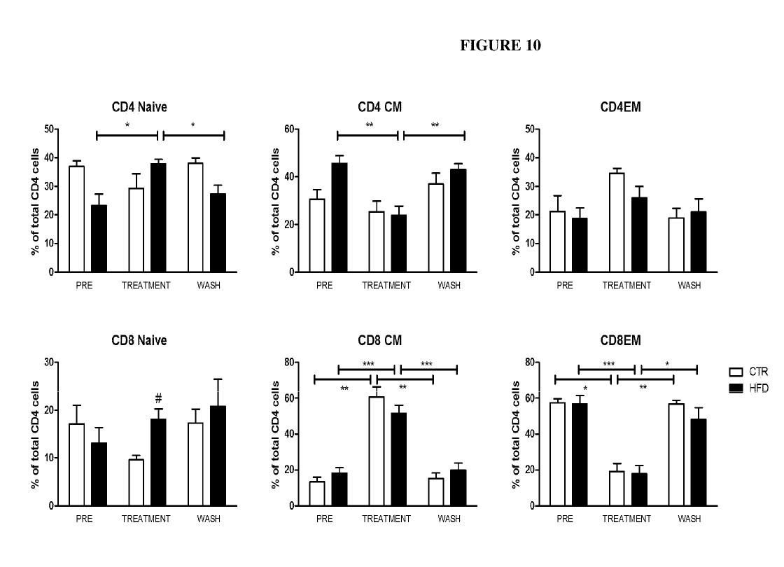

T-cell phenotyping showed a shift in CD8 positive T-cell subpopulations from effector memory to central memory following 12 weeks of 2D03 treatment. Approximately 60% of CD8 positive T-cells were effector memory and 20% central memory pre-treatment versus 20% effector memory and 60% central memory post-treatment. There was also a shift in the CD4 subpopulation from central memory to naive during 2D03 treatment. These shifts were reversed by the end of the washout period. A paired T test calculation uncorrected for multiple measures showed these population shifts to be significant (FIG. 10).

Pharmacokinetic Analysis

2D03 exhibits a biphasic profile with an initial distribution phase followed by an elimination phase. 2D03 accumulation was observed and the steady state was reached by Day 63. The mean accumulation ratio was calculated as 2.70 for the HFD group and 4.58 for the control group. The mean elimination half life (t_{1/2}) was calculated as 13.7±2.50 days (9.38-16.5 days) for HFD monkeys and 11.6±3.78 days (8.47-15.8 days) for the normal diet monkeys. AUC at steady state (AUC_{τ}) was similar for all monkeys and was approximately 843 and 680 day·μg/mL for HFD and normal diet monkeys, respectively. CLₛₛ was calculated as 12.2 mL/day/kg for HFD monkeys and 15.6 mL/day/kg for normal diet monkeys. Vₛₛ was 275 mL/kg for HFD monkeys and 405 mL/kg for normal diet monkeys.

Because antibodies to 2D03 were detected in the serum samples from Animal 19267, which led to a decline in serum concentrations beyond Day 14, this animal was excluded from PK analysis. In addition, Animals 20169 and 21937 in the HFD group and Animals 19836 and 19843 in the control group were also positive for antibodies to 2D03 during the recovery phase, but these animals were included in the PK analysis.

### CONCLUSIONS

Rhesus monkeys on a HFD became obese and insulin resistant with elevated inflammatory cytokines. Weekly treatment with 10 mg/kg of 2D03 IV improved insulin sensitivity and “normalized” the inflammatory cytokine profile. 2D03 was shown to modulate several pro- and anti-inflammatory mediators. A reduction was seen in IL-1β, TNF-α, IL-13, IL-15, FGF-basic and notably in EN-RAGE. In contrast, GM-CSF levels were increased. This dose was well tolerated with only modest trends measured in some serum chemistry analytes.

2D03 is a full length human IgG1 antibody that binds with high affinity to the malondialdehyde oxidized apo B 100 protein portion of the LDL molecule. Taken together these data demonstrate that 2D03 is anti-inflammatory and improves insulin sensitivity in Rhesus monkeys on HFD and is consistent with the previous experimental observations supporting 2D03's therapeutic potential for the treatment of diseases and disorders such as type II diabetes.

What is claimed is:

1. A method of increasing insulin sensitivity in a subject, comprising administering to the subject an effective amount of a composition comprising an antibody that selectively binds to an epitope of oxidized low density lipoprotein (LDL).

2. The method of claim 1, wherein the epitope of oxidized LDL comprises an epitope of oxidized ApoB-100.

3. The method of claim 2, wherein the epitope of oxidized ApoB-100 is selected from the group consisting of SEQ ID NO:1-SEQ ID NO:38.

4. The method of claim 3, wherein the antibody is a monoclonal antibody.

5. The method of claim 4, wherein the monoclonal antibody is an IgG1 antibody.

6. The method of claim 5, wherein the monoclonal antibody is a humanized antibody or human antibody.

7. The method of claim 4, wherein the monoclonal antibody comprises (a) a light chain variable domain comprising (i) CDR-L1 comprising sequence CSGSNTNIGKNYVS (SEQ ID NO:39); (ii) CDR-L2 comprising sequence ANSNRPS (SEQ ID NO:40); and/or (iii) CDR-L3 comprising sequence CASWDASLNGWV (SEQ ID NO:41) and (b) a heavy chain variable domain comprising (i) CDR-H1 comprising sequence FSNAWMSWVRQAPG (SEQ ID NO:42); (ii) CDR-H2 comprising sequence SSISVGGHRTYYADSVKGR (SEQ ID NO:43); and/or (iii) CDR-H3 comprising sequence ARIRVGPSGGAFDY (SEQ ID NO:44).

8. The method of claim 4, wherein the monoclonal antibody comprises (a) a light chain variable domain comprising (i) CDR-L1 comprising sequence CSGSSSNIGNNAVN (SEQ ID NO:45); (ii) CDR-L2 comprising sequence GNDRRPS (SEQ ID NO:46); and/or (iii) CDR-L3 comprising sequence CQTWGTGRGV (SEQ ID NO:47) and (b) a heavy chain variable domain comprising (i) CDR-H1 comprising sequence FSDYYMSWVRQAPG (SEQ ID NO:48); (ii) CDR-H2 comprising sequence SGVSWNGSRTHYADSVKGR (SEQ ID NO:49); and/or (iii) CDR-H3 comprising sequence ARAARYSYYYYGMDV (SEQ ID NO:50).

9. The method of claim 4, wherein the monoclonal antibody comprises (a) a light chain variable domain comprising (i) CDR-L1 comprising sequence CSGSSSNIGNNYVS (SEQ ID NO:127); (ii) CDR-L2 comprising sequence SNNQRPS (SEQ ID NO:128); and (iii) CDR-L3 comprising sequence CAAWDDSLSHWL (SEQ ID NO:129) and (b) a heavy chain variable domain comprising (i) CDR-H1 comprising sequence FSNAWMSWVRQVPG (SEQ ID NO:130); (ii) CDR-H2 comprising sequence STLGGSGGGSTYYADSVKGR (SEQ ID NO:131); and (iii) CDR-H3 comprising sequence AKLGGRSRYGRWPRQFDY (SEQ ID NO:132).

10. The method of claim 4, wherein the monoclonal antibody comprises (a) a light chain variable domain comprising (i) CDR-L1 comprising sequence CSGSSSNIGSNYVS (SEQ ID NO:133); (ii) CDR-L2 comprising sequence GNYNRPS (SEQ ID NO:134); and (iii) CDR-L3 comprising sequence CAAWDDSLSGWV (SEQ ID NO:135) and (b) a heavy chain variable domain comprising (i) CDR-H1 comprising sequence FSSYWMSWVRQAPG (SEQ ID NO:136); (ii) CDR-H2 comprising sequence SSISGSGRRTYYADSVQGR (SEQ ID NO:137); and (iii) CDR-H3 comprising sequence ARLVSYGSGSFGFDY (SEQ ID NO:138).

11. The method of claim 4, wherein the monoclonal antibody comprises (a) a light chain variable domain comprising (i) CDR-L1 comprising sequence CSGRSSNIGNSYVS (SEQ ID NO:139); (ii) CDR-L2 comprising sequence RNNQRPS (SEQ ID NO:140); and (iii) CDR-L3 comprising sequence CAGWDDTLRAWV (SEQ ID NO:141) and (b) a heavy chain variable domain comprising (i) CDR-H1 comprising sequence FRDYYVSWIRQAPG (SEQ ID NO:142); (ii) CDR-H2 comprising sequence SSISGSGGRTYYADSVEGR (SEQ ID NO:143); and (iii) CDR-H3 comprising sequence ARVSALRRPMTTVTTYWFDP (SEQ ID NO:144).

12. The method of claim 6, wherein the monoclonal antibody is a human antibody and the human antibody comprises (a) a heavy chain variable domain comprising a sequence selected from the group consisting of SEQ ID NO:64, SEQ ID NO:68, SEQ ID NO:72, SEQ ID NO:76, SEQ ID NO:80, SEQ ID NO:84, SEQ ID NO:88, SEQ ID NO:92, SEQ ID NO:96, SEQ ID NO:100, SEQ ID NO:104, SEQ ID NO:108, SEQ ID NO:112, SEQ ID NO:116, SEQ ID NO:120, and SEQ ID NO:124 and (b) a light chain variable domain comprising a sequence selected from the group consisting of SEQ ID NO:66, SEQ ID NO:70, SEQ ID NO:74, SEQ ID NO:78, SEQ ID NO:82, SEQ ID NO:86, SEQ ID NO:90, SEQ ID NO:94, SEQ ID NO:98, SEQ ID NO:102, SEQ ID NO:106, SEQ ID NO:110, SEQ ID NO:114, SEQ ID NO:118, SEQ ID NO:122, and SEQ ID NO:126.

13. The method of claim 1, wherein the antibody is an antigen binding fragment.

14. The method of claim 13, wherein the antigen binding fragment is selected from the group consisting of a Fab fragment, a Fab′ fragment, a F(ab′)₂ fragment, a scFv, a Fv, and a diabody.

15. The method of claim 1, wherein the antibody further reduces inflammation.

16. The method of claim 1, wherein the antibody further reduces levels of an inflammatory marker, wherein the inflammatory marker is selected from the group consisting of IL-1β, IL-15, EN-RAGE, MCP-1, IL-6, and TNF-α.

17. The method of claim 1, wherein the subject has metabolic syndrome or is at risk for developing metabolic syndrome.

18. The method of claim 1, wherein the subject has pre-diabetes or diabetes.

19. The method of claim 1, wherein the subject has a cardiovascular disease or coronary heart disease.

20. The method of claim 1, further comprising administering a second therapeutic agent.

21. The method, the use, the medicament, or the antibody of claim 20, wherein the second therapeutic agent is insulin.

22. The method, the use, the medicament, or the antibody of claim 20, wherein the second therapeutic agent is a statin.


SEQUENCE LISTING (144 sequences)

SEQ ID NO: 1 - protein, 20 aa, Artificial Sequence
  Synthetic construct
FLDTVYGNCSTHFTVKTRKG

SEQ ID NO: 2 - protein, 20 aa, Artificial Sequence
  Synthetic construct
PQCSTHILQWLKRVHANPLL

SEQ ID NO: 3 - protein, 20 aa, Artificial Sequence
  Synthetic construct
VISIPRLQAEARSEILAHWS

SEQ ID NO: 4 - protein, 20 aa, Artificial Sequence
  Synthetic construct
KLVKEALKESQLPTVMDFRK

SEQ ID NO: 5 - protein, 20 aa, Artificial Sequence
  Synthetic construct
LKFVTQAEGAKQTEATMTFK

SEQ ID NO: 6 - protein, 20 aa, Artificial Sequence
  Synthetic construct
DGSLRHKFLDSNIKFSHVEK

SEQ ID NO: 7 - protein, 20 aa, Artificial Sequence
  Synthetic construct
KGTYGLSCQRDPNTGRLNGE

SEQ ID NO: 8 - protein, 20 aa, Artificial Sequence
  Synthetic construct
RLNGESNLRFNSSYLQGTNQ

SEQ ID NO: 9 - protein, 20 aa, Artificial Sequence
  Synthetic construct
SLTSTSDLQSGIIKNTASLK

SEQ ID NO: 10 - protein, 20 aa, Artificial Sequence
  Synthetic construct
TASLKYENYELTLKSDTNGK

SEQ ID NO: 11 - protein, 20 aa, Artificial Sequence
  Synthetic construct
DMTFSKQNALLRSEYQADYE

SEQ ID NO: 12 - protein, 20 aa, Artificial Sequence
  Synthetic construct
MKVKIIRTIDQMQNSELQWP

SEQ ID NO: 13 - protein, 20 aa, Artificial Sequence
  Synthetic construct
IALDDAKINFNEKLSQLQTY

SEQ ID NO: 14 - protein, 20 aa, Artificial Sequence
  Synthetic construct
KTTKQSFDLSVKAQYKKNKH

SEQ ID NO: 15 - protein, 20 aa, Artificial Sequence
  Synthetic construct
EEEMLENVSLVCPKDATRFK

SEQ ID NO: 16 - protein, 20 aa, Artificial Sequence
  Synthetic construct
GSTSHHLVSRKSISAALEHK

SEQ ID NO: 17 - protein, 20 aa, Artificial Sequence
  Synthetic construct
IENIDFNKSGSSTASWIQNV

SEQ ID NO: 18 - protein, 20 aa, Artificial Sequence
  Synthetic construct
IREVTQRLNGEIQALELPQK

SEQ ID NO: 19 - protein, 20 aa, Artificial Sequence
  Synthetic construct
EVDVLTKYSQPEDSLIPFFE

SEQ ID NO: 20 - protein, 20 aa, Artificial Sequence
  Synthetic construct
HTFLIYITELLKKLQSTTVM

SEQ ID NO: 21 - protein, 20 aa, Artificial Sequence
  Synthetic construct
LLDIANYLMEQIQDDCTGDE

SEQ ID NO: 22 - protein, 20 aa, Artificial Sequence
  Synthetic construct
CTGDEDYTYLILRVIGNMGQ

SEQ ID NO: 23 - protein, 20 aa, Artificial Sequence
  Synthetic construct
GNMGQTMEQLTPELKSSILK

SEQ ID NO: 24 - protein, 20 aa, Artificial Sequence
  Synthetic construct
SSILKCVQSTKPSLMIQKAA

SEQ ID NO: 25 - protein, 20 aa, Artificial Sequence
  Synthetic construct
IQKAAIQALRKMEPKDKDQE

SEQ ID NO: 26 - protein, 20 aa, Artificial Sequence
  Synthetic construct
RLNGESNLRFNSSYLQGTNQ

SEQ ID NO: 27 - protein, 20 aa, Artificial Sequence
  Synthetic construct
SLNSHGLELNADILGTDKIN

SEQ ID NO: 28 - protein, 20 aa, Artificial Sequence
  Synthetic construct
WIQNVDTKYQIRIQIQEKLQ

SEQ ID NO: 29 - protein, 20 aa, Artificial Sequence
  Synthetic construct
TYISDWWTLAAKNLTDFAEQ

SEQ ID NO: 30 - protein, 20 aa, Artificial Sequence
  Synthetic construct
EATLQRIYSLWEHSTKNHLQ

SEQ ID NO: 31 - protein, 20 aa, Artificial Sequence
  Synthetic construct
ALLVPPETEEAKQVLFLDTV

SEQ ID NO: 32 - protein, 20 aa, Artificial Sequence
  Synthetic construct
IEIGLEGKGFEPTLEALFGK

SEQ ID NO: 33 - protein, 20 aa, Artificial Sequence
  Synthetic construct
SGASMKLTTNGRFREHNAKF

SEQ ID NO: 34 - protein, 20 aa, Artificial Sequence
  Synthetic construct
NLIGDFEVAEKINAFRAKVH

SEQ ID NO: 35 - protein, 20 aa, Artificial Sequence
  Synthetic construct
GHSVLTAKGMALFGEGKAEF

SEQ ID NO: 36 - protein, 20 aa, Artificial Sequence
  Synthetic construct
FKSSVITLNTNAELFNQSDI

SEQ ID NO: 37 - protein, 20 aa, Artificial Sequence
  Synthetic construct
FPDLGQEVALNANTKNQKIR

SEQ ID NO: 38 - protein, 20 aa, Artificial Sequence
  Synthetic construct
ATRFKHLRKYTYNYEAESSS

SEQ ID NO: 39 - protein, 14 aa, Artificial Sequence
  Synthetic construct
CSGSNTNIGKNYVS

SEQ ID NO: 40 - protein, 7 aa, Artificial Sequence
  Synthetic construct
ANSNRPS

SEQ ID NO: 41 - protein, 12 aa, Artificial Sequence
  Synthetic construct
CASWDASLNGWV

SEQ ID NO: 42 - protein, 14 aa, Artificial Sequence
  Synthetic construct
FSNAWMSWVRQAPG

SEQ ID NO: 43 - protein, 19 aa, Artificial Sequence
  Synthetic construct
SSISVGGHRTYYADSVKGR

SEQ ID NO: 44 - protein, 14 aa, Artificial Sequence
  Synthetic construct
ARIRVGPSGGAFDY

SEQ ID NO: 45 - protein, 14 aa, Artificial Sequence
  Synthetic construct
CSGSSSNIGNNAVN

SEQ ID NO: 46 - protein, 7 aa, Artificial Sequence
  Synthetic construct
GNDRRPS

SEQ ID NO: 47 - protein, 10 aa, Artificial Sequence
  Synthetic construct
CQTWGTGRGV

SEQ ID NO: 48 - protein, 14 aa, Artificial Sequence
  Synthetic construct
FSDYYMSWVRQAPG

SEQ ID NO: 49 - protein, 19 aa, Artificial Sequence
  Synthetic construct
SGVSWNGSRTHYADSVKGR

SEQ ID NO: 50 - protein, 15 aa, Artificial Sequence
  Synthetic construct
ARAARYSYYYYGMDV

SEQ ID NO: 51 - protein, 14 aa, Artificial Sequence
  Synthetic construct
CSGSSSSIGNNFVS

SEQ ID NO: 52 - protein, 7 aa, Artificial Sequence
  Synthetic construct
DNNKRPS

SEQ ID NO: 53 - protein, 12 aa, Artificial Sequence
  Synthetic construct
CAAWDDSLNGWV

SEQ ID NO: 54 - protein, 14 aa, Artificial Sequence
  Synthetic construct
FSNAWMSWVRQAPG

SEQ ID NO: 55 - protein, 19 aa, Artificial Sequence
  Synthetic construct
SSISTSSNYIYYADSVKGR

SEQ ID NO: 56 - protein, 18 aa, Artificial Sequence
  Synthetic construct
ARVKKYSSGWYSNYAFDI

SEQ ID NO: 57 - protein, 14 aa, Artificial Sequence
  Synthetic construct
CSGSSSNIGGESVS

SEQ ID NO: 58 - protein, 7 aa, Artificial Sequence
  Synthetic construct
SNNQRPS

SEQ ID NO: 59 - protein, 12 aa, Artificial Sequence
  Synthetic construct
CAAWDDSLNGWV

SEQ ID NO: 60 - protein, 14 aa, Artificial Sequence
  Synthetic construct
FSSYAMSWVRQAPG

SEQ ID NO: 61 - protein, 19 aa, Artificial Sequence
  Synthetic construct
SSISSSGRFIYYADSMKGR

SEQ ID NO: 62 - protein, 14 aa, Artificial Sequence
  Synthetic construct
TRLRRGSYFWAFDI

SEQ ID NO: 63 - DNA, 369 bp, Artificial Sequence
  Synthetic construct
gaggtgcagctgttggagtctgggggaggcttggtacagcctggggggtccctgagactctcctgtgcagcctctggattcaccttcaataacgcctggatgagctgggtccgccaggctccagggaaggggctggagtgggtctcatccattagtagtagtagtagttacatatactacgcagactcagtgaagggccgattcaccatctccagagacaattccaagaacacgctgtatctgcaaatgaacagcctgagagccgaggacactgccgtgtattactgtgcgagagtcagtaggtactactacggaccatctttctactttgactcctggggccagggtacactggtcaccgtgagcagc

SEQ ID NO: 64 - protein, 123 aa, Artificial Sequence
  Synthetic construct
EVQLLESGGGLVQPGGSLRLSCAASGFTFNNAWMSWVRQAPGKGLEWVSSISSSSSYIYYADSVKGRFTISRDNSKNTLYLQMNSLRAEDTAVYYCARVSRYYYGPSFYFDSWGQGTLVTVSS

SEQ ID NO: 65 - DNA, 336 bp, Artificial Sequence
  Synthetic construct
cagtctgtgctgactcagccaccctcagcgtctgggacccccgggcagagggtcaccatctcctgctctggaagcaggtccaacattgggaataattatgtatcctggtatcagcagctcccaggaacggcccccaaactcctcatctatggtaacaacaatcggccctcaggggtccctgaccgattctctggctccaagtctggcacctcagcctccctggccatcagtgggctccggtccgaggatgaggctgattattactgtgcagcatgggatgacagcctgaatggtcattgggtgttcggcggaggaaccaagctgacggtcctaggt

SEQ ID NO: 66 - protein, 112 aa, Artificial Sequence
  Synthetic construct
QSVLTQPPSASGTPGQRVTISCSGSRSNIGNNYVSWYQQLPGTAPKLLIYGNNNRPSGVPDRFSGSKSGTSASLAISGLRSEDEADYYCAAWDDSLNGHWVFGGGTKLTVLG

SEQ ID NO: 67 - DNA, 366 bp, Artificial Sequence
  Synthetic construct
gaggtgcagctgttggagtctgggggaggcttggtacagcctggggggtccctgagactctcctgtgcggcctctggattcaccttcagtgactactacatgagctgggtccgccaggctcccgggaaggggctggagtgggtatcgggtgttagttggaatggcagtaggacgcactatgcagactctgtgaagggccgattcaccatctccagagacaattccaagaacacgctgtatctgcaaatgaacagcctgagagccgaggacactgccgtgtattactgtgcgagagcggctaggtactcctactactactacggtatggacgtctggggccaaggtacactggtcaccgtgagcagc

SEQ ID NO: 68 - protein, 122 aa, Artificial Sequence
  Synthetic construct
EVQLLESGGGLVQPGGSLRLSCAASGFTFSDYYMSWVRQAPGKGLEWVSGVSWNGSRTHYADSVKGRFTISRDNSKNTLYLQMNSLRAEDTAVYYCARAARYSYYYYGMDVWGQGTLVTVSS

SEQ ID NO: 69 - DNA, 327 bp, Artificial Sequence
  Synthetic construct
cagtctgtgctgactcagccaccctcagcgtctgggacccccgggcagagggtcaccatctcctgttctggaagcagctccaacatcggaaataatgctgtaaactggtatcagcagctcccaggaacggcccccaaactcctcatctatgggaatgatcggcggccctcaggggtccctgaccgattctctggctccaagtctggcacctcagcctccctggccatcagtgggctccggtccgaggatgaggctgattattactgtcagacctggggcactggccggggggtattcggcggaggaaccaagctgacggtcctaggt

SEQ ID NO: 70 - protein, 109 aa, Artificial Sequence
  Synthetic construct
QSVLTQPPSASGTPGQRVTISCSGSSSNIGNNAVNWYQQLPGTAPKLLIYGNDRRPSGVPDRFSGSKSGTSASLAISGLRSEDEADYYCQTWGTGRGVFGGGTKLTVLG

SEQ ID NO: 71 - DNA, 366 bp, Artificial Sequence
  Synthetic construct
gaggtgcagctgttggagtctgggggaggcttggtacagcctggggggtccctgagactctcctgtgcagcctctggattcacctttagtagctattggatgagctgggtccgccaggctccagggaaggggctggagtgggtctcaagtatcagtggtagtggtcgtaggacatactacgcagactccgtgcagggccggttcaccatctccagagacaattccaagaacacgctgtatctgcaaatgaacagcctgagagccgaggacactgccgtgtattactgtgcgagattggtctcctatggttcggggagtttcggttttgactactggggccaaggtacactggtcaccgtgagcagc

SEQ ID NO: 72 - protein, 122 aa, Artificial Sequence
  Synthetic construct
EVQLLESGGGLVQPGGSLRLSCAASGFTFSSYWMSWVRQAPGKGLEWVSSISGSGRRTYYADSVQGRFTISRDNSKNTLYLQMNSLRAEDTAVYYCARLVSYGSGSFGFDYWGQGTLVTVSS

SEQ ID NO: 73 - DNA, 333 bp, Artificial Sequence
  Synthetic construct
cagtctgtgctgactcagccaccctcagcgtctgggacccccgggcagagggtcaccatctcttgttctggaagcagctccaatatcggaagtaattatgtatcctggtatcagcagctcccaggaacggcccccaaactcctcatctatggtaactacaatcggccctcaggggtccctgaccgattctctggctccaagtctggcacctcagcctccctggccatcagtgggctccggtccgaggatgaggctgattattactgtgcagcatgggatgacagcctgagtggttgggtgttcggcggaggaaccaagctgacggtcctaggt

SEQ ID NO: 74 - protein, 110 aa, Artificial Sequence
  Synthetic construct
QSVLTQPPSASGTPGQRVTISCSGSSSNIGSNYVSWYQQLPGTAPKLLIYGNYNRPSGVPDRFSGSKSGTSASLAISGLRSEDEADYYCAAWDDSLSGWVFGGGTKLTVL

SEQ ID NO: 75 - DNA, 378 bp, Artificial Sequence
  Synthetic construct
gaggtgcagctgttggagtctgggggaggcttggtacagcctggggggtccctgagactctcctgtgcagcctctggattcaccttcagtaacgcctggatgagctgggtccgccaggttccagggaaggggctggagtgggtctcaactcttggtggtagtggtggtggtagcacatactacgcagactccgtgaagggccggttcaccatctccagagacaattccaagaacacgctgtatctgcaaatgaacagcctgagagccgaggacactgccgtgtattactgtgcgaagttaggggggcgatcccgatatgggcggtggccccgccaatttgactactggggccaaggtacactggtcaccgtgagcagc

SEQ ID NO: 76 - protein, 126 aa, Artificial Sequence
  Synthetic construct
EVQLLESGGGLVQPGGSLRLSCAASGFTFSNAWMSWVRQVPGKGLEWVSTLGGSGGGSTYYADSVKGRFTISRDNSKNTLYLQMNSLRAEDTAVYYCAKLGGRSRYGRWPRQFDYWGQGTLVTVSS

SEQ ID NO: 77 - DNA, 333 bp, Artificial Sequence
  Synthetic construct
cagtctgtgctgactcagccaccctcagcgtctgggacccccgggcagagggtcaccatctcctgctctggaagcagctccaacattggaaataactatgtatcctggtatcagcagctcccaggaacggcccccaaactcctcatctatagtaataatcagcggccctcaggggtccctgaccgattctctggctccaagtctggcacctcagcctccctggccatcagtgggctccggtccgaggatgaggctgattattactgtgcagcatgggatgacagcctgagtcattggctgttcggcggaggaaccaagctgacggtcctaggt

SEQ ID NO: 78 - protein, 110 aa, Artificial Sequence
  Synthetic construct
QSVLTQPPSASGTPGQRVTISCSGSSSNIGNNYVSWYQQLPGTAPKLLIYSNNQRPSGVPDRFSGSKSGTSASLAISGLRSEDEADYYCAAWDDSLSHWLFGGGTKLTVL

SEQ ID NO: 79 - DNA, 372 bp, Artificial Sequence
  Synthetic construct
gaggtgcagctgttggagtctgggggaggcttggtacagcctggggggtccctgagactctcctgtgcagcctctggattcaccttcagtgactactacatgagctggatccgccaggctccagggaaggggctggagtgggtctcaagtatcagtggccgtgggggtagttcctactacgcagactccgtgaggggccggttcaccatctccagagacaattccaagaacacgctgtatctgcaaatgaacagcctgagagccgaggacactgccgtgtattactgtgcgagactttcctacagctatggttacgagggggcctactactttgactactggggccagggtacactggtcaccgtgagcagc

SEQ ID NO: 80 - protein, 124 aa, Artificial Sequence
  Synthetic construct
EVQLLESGGGLVQPGGSLRLSCAASGFTFSDYYMSWIRQAPGKGLEWVSSISGRGGSSYYADSVRGRFTISRDNSKNTLYLQMNSLRAEDTAVYYCARLSYSYGYEGAYYFDYWGQGTLVTVSS

SEQ ID NO: 81 - DNA, 333 bp, Artificial Sequence
  Synthetic construct
cagtctgtgctgactcagccaccctcagcgtctgggacccccgggcagagggtcaccatctcctgctctggaagcagctccaacattgggaataattatgtatcctggtatcagcagctcccaggaacggcccccaaactcctcatctataggaataatcagcggccctcaggggtccctgaccgattctctggctccaagtctggcaccttagcctccctggccatcagtgggctccggtccgaggatgaggctgattattactgtgcaacctgggatgacagcctgaatggttgggtgttcggcggaggaaccaagctgacggtcctaggt

SEQ ID NO: 82 - protein, 111 aa, Artificial Sequence
  Synthetic construct
QSVLTQPPSASGTPGQRVTISCSGSSSNIGNNYVSWYQQLPGTAPKLLIYRNNQRPSGVPDRFSGSKSGTLASLAISGLRSEDEADYYCATWDDSLNGWVFGGGTKLTVLG

SEQ ID NO: 83 - DNA, 363 bp, Artificial Sequence
  Synthetic construct
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcatccattagtagtagtggtcgtttcatttactacgcagactcaatgaagggccgcttcaccatctccagagacaattccaagaacacgctgtatctgcaaatgaacagcctgagagccgaggacactgccgtgtattactgtacgaggctccggagagggagctacttctgggcttttgatatctggggccaaggtacactggtcaccgtgagcagc

SEQ ID NO: 84 - protein, 121 aa, Artificial Sequence
  Synthetic construct
EVQLLESGGGLVQPGGSLRLSCAASGFTFSSYAMSWVRQAPGKGLEWVSSISSSGRFIYYADSMKGRFTISRDNSKNTLYLQMNSLRAEDTAVYYCTRLRRGSYFWAFDIWGQGTLVTVSS

SEQ ID NO: 85 - DNA, 333 bp, Artificial Sequence
  Synthetic construct
cagtctgtgctgactcagccaccctcagcgtctgggacccccgggcagagggtcaccatctcctgttctggaagcagctccaacattggcggtgagtctgtatcctggtatcagcagctcccaggaacggcccccaaactcctcatctatagtaataatcagcggccctcaggggtccctgaccgattctctggctccaagtctggcacctcagcctccctggccatcagtgggctccggtccgaggatgaggctgattattactgtgcagcatgggatgacagcctgaatggttgggtgttcggcggaggaaccaagctgacggtcctaggt

SEQ ID NO: 86 - protein, 111 aa, Artificial Sequence
  Synthetic construct
QSVLTQPPSASGTPGQRVTISCSGSSSNIGGESVSWYQQLPGTAPKLLIYSNNQRPSGVPDRFSGSKSGTSASLAISGLRSEDEADYYCAAWDDSLNGWVFGGGTKLTVLG

SEQ ID NO: 87 - DNA, 369 bp, Artificial Sequence
  Synthetic construct
gaggtgcagctgttggagtctgggggaggcttggtacagcctggggggtccctgagactctcctgtgcagcctctggattcacctttagaacgtattggatgacctgggtccgccaggctccagggaaggggctggagtgggtctcatctattagcagtagcagtaattacatattctacgcagactcagtgaagggccgattcaccatctccagagacaattccaagaacacgctgtatctgcaaatgaacagcctgagagccgaggacactgccgtgtattactgtgcgagactcagacggagcagctggtacggggggtactggttcgacccctggggccaaggtacactggtcaccgtgagctca

SEQ ID NO: 88 - protein, 123 aa, Artificial Sequence
  Synthetic construct
EVQLLESGGGLVQPGGSLRLSCAASGFTFRTYWMTWVRQAPGKGLEWVSSISSSSNYIFYADSVKGRFTISRDNSKNTLYLQMNSLRAEDTAVYYCARLRRSSWYGGYWFDPWGQGTLVTVSS

SEQ ID NO: 89 - DNA, 336 bp, Artificial Sequence
  Synthetic construct
cagtctgtgctgactcagccaccctcagcgtctgggacccccgggcagagggtcaccatctcctgctctggaagcagctccaacattgggaataattatgtatcctggtatcagcagctcccaggaacggcccccaaactcctcatctataggaataatcagcggccctcaggggtccctgaccgattctctggctccaagtctggcacctcagcctccctggccatcagtgggctccggtccgaggatgaggctgattattactgtgcagcatgggatgacagcctgaatggtcattgggtgttcggcggaggaaccaagctgacggtcctaggt

SEQ ID NO: 90 - protein, 112 aa, Artificial Sequence
  Synthetic construct
QSVLTQPPSASGTPGQRVTISCSGSSSNIGNNYVSWYQQLPGTAPKLLIYRNNQRPSGVPDRFSGSKSGTSASLAISGLRSEDEADYYCAAWDDSLNGHWVFGGGTKLTVLG

SEQ ID NO: 91 - DNA, 369 bp, Artificial Sequence
  Synthetic construct
gaggtgcagctgttggagtctgggggaggcttggtacagcctggggggtccctgagactctcctgtgcagcctctggattcaccttcagtagcaactacatgagctgggtccgccaggctccagggaaggggctggagtgggtctcatccattagtagtagtagtagttacatatactacgcagactcagtgaagggccgattcaccatctccagagacaattccaagaacacgctgtatctgcaaatgaacagcctgagagccgaggacactgccgtgtattactgtgcgagagtaggccggtataactggaagacggggcatgcttttgatatctggggccagggtacactggtcaccgtgagctca

SEQ ID NO: 92 - protein, 123 aa, Artificial Sequence
  Synthetic construct
EVQLLESGGGLVQPGGSLRLSCAASGFTFSSNYMSWVRQAPGKGLEWVSSISSSSSYIYYADSVKGRFTISRDNSKNTLYLQMNSLRAEDTAVYYCARVGRYNWKTGHAFDIWGQGTLVTVSS

SEQ ID NO: 93 - DNA, 333 bp, Artificial Sequence
  Synthetic construct
cagtctgtgctgactcagccaccctcagcgtctgggacccccgggcagagggtcaccatctcctgctctggaaggacctacaacattggaaataattatgtatcgtggtatcagcagctcccaggaacggcccccaaactcctcatctatggtaacatcaatcggccctcaggggtccctgaccgattctctggctccaagtctggcacctcagcctccctggccatcagtgggctccggtccgaggatgaggctgattattactgtgcagcatgggatgtcaggctgaatggttgggtgttcggcggaggaaccaagctgacggtcctaggt

SEQ ID NO: 94 - protein, 111 aa, Artificial Sequence
  Synthetic construct
QSVLTQPPSASGTPGQRVTISCSGRTYNIGNNYVSWYQQLPGTAPKLLIYGNINRPSGVPDRFSGSKSGTSASLAISGLRSEDEADYYCAAWDVRLNGWVFGGGTKLTVLG

SEQ ID NO: 95 - DNA, 381 bp, Artificial Sequence
  Synthetic construct
gaggtgcagctgttggagtctgggggaggcttggtacagcctggggggtccctgagactctcctgtgcagcctctggattcaccttccgtgactactacgtgagctggatccgccaggctccagggaaggggctggagtgggtctcaagtattagtggtagtgggggtaggacatactacgcagactccgtggagggccggttcaccatctccagagacaattccaagaacacgctgtatctgcaaatgaacagcctgagagccgaggacactgccatgtattactgtgccagagtatccgcccttcggagacccatgactacagtaactacttactggttcgacccctggggccaaggtacactggtcaccgtgagctca

SEQ ID NO: 96 - protein, 127 aa, Artificial Sequence
  Synthetic construct
EVQLLESGGGLVQPGGSLRLSCAASGFTFRDYYVSWIRQAPGKGLEWVSSISGSGGRTYYADSVEGRFTISRDNSKNTLYLQMNSLRAEDTAMYYCARVSALRRPMTTVTTYWFDPWGQGTLVTVSS

SEQ ID NO: 97 - DNA, 333 bp, Artificial Sequence
  Synthetic construct
cagtctgtgctgactcagccaccctcagcgtctgggacccccgggcagagggtcaccatctcctgctctggaaggagctccaacattgggaatagttatgtctcctggtatcagcagctcccaggaacggcccccaaactcctcatctataggaataatcagcggccctcaggggtccctgaccgattctctggctccaagtctggcacctcagcctccctggccatcagtgggctccggtccgaggatgaggctgattattactgtgcaggatgggatgacaccctgcgtgcttgggtgttcggcggaggaaccaagctgacggtcctaggt

SEQ ID NO: 98 - protein, 111 aa, Artificial Sequence
  Synthetic construct
QSVLTQPPSASGTPGQRVTISCSGRSSNIGNSYVSWYQQLPGTAPKLLIYRNNQRPSGVPDRFSGSKSGTSASLAISGLRSEDEADYYCAGWDDTLRAWVFGGGTKLTVLG

SEQ ID NO: 99 - DNA, 360 bp, Artificial Sequence
  Synthetic construct
gaggtgcagctgttggagtctgggggaggcttggtacagcctggggggtccctgagactctcctgtgcagcctctggattcaccttcagtaacgcctggatgagctgggtccgccaggctccagggaaggggctggagtgggtctccgctattagtggtagtggtaacacatactatgcagactccgtgaagggccggttcaccatctccagagacaattccaagaacacgctgtatctgcaaatgaacagcctgagagccgaggacactgccgtgtattactgtgcgagagcctcccaccgtatattaggttatgcttttgatatctggggccagggtacactggtcaccgtgagctca

SEQ ID NO: 100 - protein, 120 aa, Artificial Sequence
  Synthetic construct
EVQLLESGGGLVQPGGSLRLSCAASGFTFSNAWMSWVRQAPGKGLEWVSAISGSGNTYYADSVKGRFTISRDNSKNTLYLQMNSLRAEDTAVYYCARASHRILGYAFDIWGQGTLVTVSS

SEQ ID NO: 101 - DNA, 328 bp, Artificial Sequence
  Synthetic construct
cagtctgtgctgactcagccaccctcagcgtctgggacccccgggcagagggtcaccatctcttgttctggaagccgctccaacatcgggagaaatgctgttagttggtatcagcagctcccaggaacggcccccaaactcctcatctatgctaacagcaatcggccctcaggggtccctgaccgattctctggctccaagtctggcacctcagcctccctggccatcagtgggctccggtccgaggatgaggctgattattactgtgcagcatgggatggcagcctgaatggttgggtgttcggcggaggaaccaagctgacggtcc

SEQ ID NO: 102 - protein, 109 aa, Artificial Sequence
  Synthetic construct
QSVLTQPPSASGTPGQRVTISCSGSRSNIGRNAVSWYQQLPGTAPKLLIYANSNRPSGVPDRFSGSKSGTSASLAISGLRSEDEADYYCAAWDGSLNGWVFGGGTKLTV

SEQ ID NO: 103 - DNA, 363 bp, Artificial Sequence
  Synthetic construct
gaggtgcagctgttggagtctgggggaggcttggtacagcctggggggtccctgagactctcctgtgcagcctctggattcaccttcagtaacgcctggatgagctgggtccgccaggctccagggaaggggctggagtgggtctcaagtattagtgttggtggacataggacatattatgcagattccgtgaagggccggtccaccatctccagagacaattccaagaacacgctgtatctgcaaatgaacagcctgagagccgaggacactgccgtgtattactgtgcacggatacgggtgggtccgtccggcggggcctttgactactggggccagggtacactggtcaccgtgagctca

SEQ ID NO: 104 - protein, 121 aa, Artificial Sequence
  Synthetic construct
EVQLLESGGGLVQPGGSLRLSCAASGFTFSNAWMSWVRQAPGKGLEWVSSISVGGHRTYYADSVKGRSTISRDNSKNTLYLQMNSLRAEDTAVYYCARIRVGPSGGAFDYWGQGTLVTVSS

SEQ ID NO: 105 - DNA, 333 bp, Artificial Sequence
  Synthetic construct
cagtctgtgctgactcagccaccctcagcgtctgggacccccgggcagagggtcaccatctcctgctctggaagcaacaccaacattgggaagaactatgtatcttggtatcagcagctcccaggaacggcccccaaactcctcatctatgctaatagcaatcggccctcaggggtccctgaccgattctctggctccaagtctggcacctcagcctccctggccatcagtgggctccggtccgaggatgaggctgattattactgtgcgtcatgggatgccagcctgaatggttgggtattcggcggaggaaccaagctgacggtcctaggt

SEQ ID NO: 106 - protein, 111 aa, Artificial Sequence
  Synthetic construct
QSVLTQPPSASGTPGQRVTISCSGSNTNIGKNYVSWYQQLPGTAPKLLIYANSNRPSGVPDRFSGSKSGTSASLAISGLRSEDEADYYCASWDASLNGWVFGGGTKLTVLG

SEQ ID NO: 107 - DNA, 378 bp, Artificial Sequence
  Synthetic construct
gaggtgcagctgttggagtctgggggaggcttggtacagcctggggggtccctgagactctcctgtgcagcctctggattcaccttcagtaacgcctggatgagctgggtccgccaggctccagggaaggggctggagtgggtctcatccattagtagtagtagtagttacatatactacgcagactcagtgaagggccgatccaccatctccagagacaattccaagaacacgctgtatctgcaaatgaacagcctgagagccgaggacactgccgtgtattactgtgcgaggctcacaaatattttgactggttattatacctcaggatatgcttttgatatctggggccaaggtacactggtcaccgtgagctca

SEQ ID NO: 108 - protein, 126 aa, Artificial Sequence
  Synthetic construct
EVQLLESGGGLVQPGGSLRLSCAASGFTFSNAWMSWVRQAPGKGLEWVSSISSSSSYIYYADSVKGRSTISRDNSKNTLYLQMNSLRAEDTAVYYCARLTNILTGYYTSGYAFDIWGQGTLVTVSS

SEQ ID NO: 109 - DNA, 333 bp, Artificial Sequence
  Synthetic construct
cagtctgtgctgactcagccaccctcagcgtctgggacccccgggcagagggtcaccatctcctgctctggaagcacctccaacattgggaagaattatgtatcctggtatcagcagctcccaggaacggcccccaaactcctcatctatggtaacagcaatcggccctcaggggtccctgaccgattctctggctccaagtctggcacctcagcctccctggccatcagtgggctccggtccgaggatgaggctgattattactgtgcagcatgggatgccagcctcagtggttgggtgttcggcggaggaaccaagctgacggtcctaggt

SEQ ID NO: 110 - protein, 111 aa, Artificial Sequence
  Synthetic construct
QSVLTQPPSASGTPGQRVTISCSGSTSNIGKNYVSWYQQLPGTAPKLLIYGNSNRPSGVPDRFSGSKSGTSASLAISGLRSEDEADYYCAAWDASLSGWVFGGGTKLTVLG

SEQ ID NO: 111 - DNA, 363 bp, Artificial Sequence
  Synthetic construct
gaggtgcagctgttggagtctgggggaggcttggtacagcctggggggtccctgagactctcctgtgcagcctctggattcaccttcagtagttcttggatgagttgggtccgccaggctccagggaaggggctggagtgggtctcatccattagtagtagtagtagttacatatactacgcagactcagtgaagggccgattcaccatctccagagacaattccaagaacacgctgtatctgcaaatgaacagcctgagagccgaggacactgccgtgtattactgtgcgagagtagggaactacggtttctaccactacatggacgtctggggccaaggtacactggtcaccgtgagctca

SEQ ID NO: 112 - protein, 121 aa, Artificial Sequence
  Synthetic construct
EVQLLESGGGLVQPGGSLRLSCAASGFTFSSSWMSWVRQAPGKGLEWVSSISSSSSYIYYADSVKGRFTISRDNSKNTLYLQMNSLRAEDTAVYYCARVGNYGFYHYMDVWGQGTLVTVSS

SEQ ID NO: 113 - DNA, 333 bp, Artificial Sequence
  Synthetic construct
cagtctgtgctgactcagccaccctcagcgtctgggacccccgggcagagggtcaccatctcttgttctggaggcagctcaaacatcggaaaaagaggtgtaaattggtatcagcagctcccaggaacggcccccaaactcctcatctatggtaacagaaatcggccctcaggggtccctgaccgattctctggctccaagtctggcacctcagcctccctggccatcagtgggctccggtccgaggatgaggctgattattactgtgctacatgggattacagcctcaatgcttgggtgttcggcggaggaaccaagctgacggtcctaggt

SEQ ID NO: 114 - protein, 111 aa, Artificial Sequence
  Synthetic construct
QSVLTQPPSASGTPGQRVTISCSGGSSNIGKRGVNWYQQLPGTAPKLLIYGNRNRPSGVPDRFSGSKSGTSASLAISGLRSEDEADYYCATWDYSLNAWVFGGGTKLTVLG

SEQ ID NO: 115 - DNA, 366 bp, Artificial Sequence
  Synthetic construct
gaggtgcagctgttggagtctgggggaggcttggtacagcctggggggtccctgagactctcctgtgcagcctctggattcacctttagtagctattggatgagctgggtccgccaggctccagggaaggggctggagtgggtctcatccattagtagtagtagtagttacatatactacgcagactcagtgaagggccgattcaccatctccagagacaattccaagaacacgctgtatctgcaaatgaacagcctgagagccgaggacactgccgtgtattactgtgcgagaattaaacggttacgattcggctggaccccttttgactactggggccagggtacactggtcaccgtgagctca

SEQ ID NO: 116 - protein, 122 aa, Artificial Sequence
  Synthetic construct
EVQLLESGGGLVQPGGSLRLSCAASGFTFSSYWMSWVRQAPGKGLEWVSSISSSSSYIYYADSVKGRFTISRDNSKNTLYLQMNSLRAEDTAVYYCARIKRLRFGWTPFDYWGQGTLVTVSS

SEQ ID NO: 117 - DNA, 333 bp, Artificial Sequence
  Synthetic construct
cagtctgttctgactcagccaccctcagcgtctgggacccccgggcagagggtcaccatctcctgttctggaagcagctccaacatcggaaataatggtgtaaactggtatcagcagctcccaggaacggcccccaaactcctcatctatggtaacaacaatcggccctcaggggtccctgaccgattctctggctccaagtctggcacctcagcctccctggccatcagtgggctccggtccgaggatgaggctgattattactgtgcagcatgggatgacagcctgcgtggttggctgttcggcggaggaaccaagctgacggtcctaggt

SEQ ID NO: 118 - protein, 111 aa, Artificial Sequence
  Synthetic construct
QSVLTQPPSASGTPGQRVTISCSGSSSNIGNNGVNWYQQLPGTAPKLLIYGNNNRPSGVPDRFSGSKSGTSASLAISGLRSEDEADYYCAAWDDSLRGWLFGGGTKLTVLG

SEQ ID NO: 119 - DNA, 369 bp, Artificial Sequence
  Synthetic construct
gaggtgcagctgttggagtctgggggaggcttggtacagcctggggggtccctgagactctcctgtgcagcctctggattcaccttcagtaacgcctggatgagctgggtccgccaggctccagggaaggggctggagtgggtctcatccattagtagtagtagtagttacatatactacgcagactcagtgaagggccgattcaccatctccagagacaattccaagaacacgctgtatctgcaaatgaacagcctgagagccgaggacactgccgtgtattactgtgcgagagtcaatagcaaaaagtggtatgagggctacttctttgactactggggccagggtacactggtcaccgtgagctca

SEQ ID NO: 120 - protein, 123 aa, Artificial Sequence
  Synthetic construct
EVQLLESGGGLVQPGGSLRLSCAASGFTFSNAWMSWVRQAPGKGLEWVSSISSSSSYIYYADSVKGRFTISRDNSKNTLYLQMNSLRAEDTAVYYCARVNSKKWYEGYFFDYWGQGTLVTVSS

SEQ ID NO: 121 - DNA, 333 bp, Artificial Sequence
  Synthetic construct
cagtctgtgctgactcagccaccctcagcgtctgggacccccgggcagagggtcaccatctcctgctctggaagcagctccaacattgggaataattatgtatcctggtatcagcagctcccaggaacggcccccaaactcctcatctatggtaacagcaatcggccctcaggggtccctgaccgattctctggctccaagtctggcacctcagcctccctggccatcagtgggctccggtccgaggatgaggctgattattactgtgcagcatgggatgacagtctgagtggttgggtgttcggcggaggaaccaagctgacggtcctaggt

SEQ ID NO: 122 - protein, 111 aa, Artificial Sequence
  Synthetic construct
QSVLTQPPSASGTPGQRVTISCSGSSSNIGNNYVSWYQQLPGTAPKLLIYGNSNRPSGVPDRFSGSKSGTSASLAISGLRSEDEADYYCAAWDDSLSGWVFGGGTKLTVLG

SEQ ID NO: 123 - DNA, 375 bp, Artificial Sequence
  Synthetic construct
gaggtgcagctgttggagtctgggggaggcttggtacagcctggggggtccctgagactctcctgtgcagcctctggattcaccttcagtaacgcctggatgagctgggtccgccaggctccagggaaggggctggagtgggtctcatccattagtactagtagtaattacatatactacgcagactcagtgaagggccggttcaccatctccagagacaattccaagaacacgctgtatctgcaaatgaacagcctgagagccgaggacactgccgtgtattactgtgcgagagtcaagaagtatagcagtggctggtactcgaattatgcttttgatatctggggccaaggtacactggtcaccgtgagctca

SEQ ID NO: 124 - protein, 125 aa, Artificial Sequence
  Synthetic construct
EVQLLESGGGLVQPGGSLRLSCAASGFTFSNAWMSWVRQAPGKGLEWVSSISTSSNYIYYADSVKGRFTISRDNSKNTLYLQMNSLRAEDTAVYYCARVKKYSSGWYSNYAFDIWGQGTLVTVSS

SEQ ID NO: 125 - DNA, 333 bp, Artificial Sequence
  Synthetic construct
cagtctgtgctgactcagccaccctcagcgtctgggacccccgggcagagggtcaccatctcctgctctggaagcagctccagcattgggaataattttgtatcctggtatcagcagctcccaggaacggcccccaaactcctcatctatgacaataataagcgaccctcaggggtccctgaccgattctctggctccaagtctggcacctcagcctccctggccatcagtgggctccggtccgaggatgaggctgattattactgtgcagcatgggatgacagcctgaatggttgggtgttcggcggaggaaccaagctgacggtcctaggt

SEQ ID NO: 126 - protein, 111 aa, Artificial Sequence
  Synthetic construct
QSVLTQPPSASGTPGQRVTISCSGSSSSIGNNFVSWYQQLPGTAPKLLIYDNNKRPSGVPDRFSGSKSGTSASLAISGLRSEDEADYYCAAWDDSLNGWVFGGGTKLTVLG

SEQ ID NO: 127 - protein, 14 aa, Artificial Sequence
  Synthetic construct
CSGSSSNIGNNYVS

SEQ ID NO: 128 - protein, 7 aa, Artificial Sequence
  Synthetic construct
SNNQRPS

SEQ ID NO: 129 - protein, 12 aa, Artificial Sequence
  Synthetic construct
CAAWDDSLSHWL

SEQ ID NO: 130 - protein, 14 aa, Artificial Sequence
  Synthetic construct
FSNAWMSWVRQVPG

SEQ ID NO: 131 - protein, 20 aa, Artificial Sequence
  Synthetic construct
STLGGSGGGSTYYADSVKGR

SEQ ID NO: 132 - protein, 18 aa, Artificial Sequence
  Synthetic construct
AKLGGRSRYGRWPRQFDY

SEQ ID NO: 133 - protein, 14 aa, Artificial Sequence
  Synthetic construct
CSGSSSNIGSNYVS

SEQ ID NO: 134 - protein, 7 aa, Artificial Sequence
  Synthetic construct
GNYNRPS

SEQ ID NO: 135 - protein, 12 aa, Artificial Sequence
  Synthetic construct
CAAWDDSLSGWV

SEQ ID NO: 136 - protein, 14 aa, Artificial Sequence
  Synthetic construct
FSSYWMSWVRQAPG

SEQ ID NO: 137 - protein, 19 aa, Artificial Sequence
  Synthetic construct
SSISGSGRRTYYADSVQGR

SEQ ID NO: 138 - protein, 15 aa, Artificial Sequence
  Synthetic construct
ARLVSYGSGSFGFDY

SEQ ID NO: 139 - protein, 14 aa, Artificial Sequence
  Synthetic construct
CSGRSSNIGNSYVS

SEQ ID NO: 140 - protein, 7 aa, Artificial Sequence
  Synthetic construct
RNNQRPS

SEQ ID NO: 141 - protein, 12 aa, Artificial Sequence
  Synthetic construct
CAGWDDTLRAWV

SEQ ID NO: 142 - protein, 14 aa, Artificial Sequence
  Synthetic construct
FRDYYVSWIRQAPG

SEQ ID NO: 143 - protein, 19 aa, Artificial Sequence
  Synthetic construct
SSISGSGGRTYYADSVEGR

SEQ ID NO: 144 - protein, 20 aa, Artificial Sequence
  Synthetic construct
ARVSALRRPMTTVTTYWFDP